SEQUENCE LISTING (107 sequences)

SEQ ID NO: 1 - protein, 5 aa, synthetic construct
SFGMH

SEQ ID NO: 2 - protein, 17 aa, synthetic construct
YISSDSFTIYYADTMKG

SEQ ID NO: 3 - protein, 7 aa, synthetic construct
GDLPFAY

SEQ ID NO: 4 - protein, 11 aa, synthetic construct
KASQAVGTAVA

SEQ ID NO: 5 - protein, 7 aa, synthetic construct
WASTRHT

SEQ ID NO: 6 - protein, 9 aa, synthetic construct
HQYSSYPLT

SEQ ID NO: 7 - protein, 135 aa, synthetic construct
MDSRLNLVFLVLILKGVQCDVQLVESGGGLVQPGGSRKLSCAASGFTFSSFGMHWVRQAPEKGLEWVAYISSDSFTIYYADTMKGRFTISRDNPQNTLFLQMTSLRSEDTAMYYCARGDLPFAYWGQGTLVTVSA

SEQ ID NO: 8 - protein, 131 aa, synthetic construct
MGIKMETHSQVFVYMLLWLSGVEGDIVMTQSHKFMSTSVGDRVSITCKASQAVGTAVAWYQQKPGQSPKLLIYWASTRHTGVPDRFTGSGSGTDFTLTISNVQSEDLADYFCHQYSSYPLTFGAGTKLELK

SEQ ID NO: 9 - protein, 373 aa, synthetic construct
RLACKEDYRYAISSTDEIGLLGAGGLTTTWKEYNHDLQLNDGTVKASCVAGSFKVIALNVVSRRYLASLHKKALPTSVTFELLFDGTNPSTEEMGDDFRSGLCPFDTSPVVKGKYNTTLLNGSAFYLVCPIGWTGVIECTAVSPTTLRTEVVKTFRRDKPFPHRMDCVTTTVENEDLFYCKLGGNWTCVKGEPVVYTGGVVKQCRWCGFDFDGPDGLPHYPIGKCILANETGYRIVDSTDCNRDGVVISTEGSHECLIGNTTVKVHASDERLGPMPCRPKEIVSSAGPVRKTSCTFNYTKTLKNRYYEPRDSYFQQYMLKGEYQYWFDLDATDRHSDYFAEFVVLVVVALLGGRYVLWLIVTYVVLTEQLAAG

SEQ ID NO: 10 - protein, 373 aa, synthetic construct
RLSCKEDYRYAISSTNEIGPLGAEGLTTTWKEYNHGLQLDDGTVRAICTAGSFKVIALNVVSRRYLASLHKRALPTSVTFELLFDGTSPAIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLDVTDHHTDYFAEFVVLVVVALLGGRYVLWLIVTYVILTEQLAAG

SEQ ID NO: 11 - protein, 373 aa, synthetic construct
RLTCKEDYRYAISSTNEIGPLGAEGLTTTWKEYNHGLQLDDGTVRATCTAGSFKVIALNVVSRRYLASLHKRALPTSVTFELLFDGTSPVIEEMGDDFGFGLCPFDTIPVVKGKYNTTLLNGSAFYLVCPIGWTGVIECTAVSPTTLRTEVVKTFKREKPFPHRVDCVTTIVEKEDLFYCRLGGNWTCVKGDPVTYTGGQVKQCRWCGFNFKEPDGLPHYPIGKCILANETGYRVVDSTDCNRDGVVIGTEGEHECLIGNTTVKVHALDGRLAPMPCRPKEIVSSAGPVRKTSCTFNYTKTLRNKYYEPRDSYFQQYMLKGEYQYWFDLDVTDHHTDYFAEFVVLVVVALLGGRYVLWLIVTYIVLTEQLAAG

SEQ ID NO: 12 - protein, 373 aa, synthetic construct
RLSCKEDYRYAISSTNEIGPLGAEGLTTTWKEYNHGLQLDDGTVRAICTAGSFKVTALNVVSRRYLASLHKRALPTSVTFELLFDGTTPAVEEMGDDFGFGLCPFDTTPVVKGKYNTTLLNGSAFYLVCPIGWTGVIECTAVSPTTLRTEVVKTFKREKPFPHRVDCVTTIVEKEDLFYCKLGGNWTCVKGNPVTYTGGQVKQCRWCGFDFKEPDGLPHYPIGKCILVNETGYRVVDSTDCNRDGVVISTEGEHECLIGNTTVKVHALDGRLAPMPCRPKEIISSAGPVRKTSCTFNYTKTLRNKYYEPRDSYFQQYMLKGEYQYWFDLDVTDHHTDYFAEFIVLVVVALLGGRYVLWLIVTYIVLTEQLAAG

SEQ ID NO: 13 - protein, 16 aa, synthetic construct
IVQGVVWLLLVTGAQG

SEQ ID NO: 14 - protein, 21 aa, synthetic construct
ILRGQVVQGIIWLLLVTGAQG

SEQ ID NO: 15 - protein, 21 aa, synthetic construct
VLRGQVVQGVIWLLLVTGAQG

SEQ ID NO: 16 - skipped

SEQ ID NO: 17 - protein, 5 aa, synthetic construct
GGGGS

SEQ ID NO: 18 - protein, 6 aa, synthetic construct
GSGGSG

SEQ ID NO: 19 - protein, 10 aa, synthetic construct
GGGGSGGGGS

SEQ ID NO: 20 - protein, 229 aa, synthetic construct
TKTKPPCPICPACESPGPSVFIFPPKPKDTLMISRTPQVTCVVVDVSQENPEVQFSWYVDGVEVHTAQTRPKEEQFNSTYRVVSVLPIQHQDWLNGKEFKCKVNNKDLPAPITRIISKAKGQTREPQVYTLPPHAEELSRSKVSITCLVIGFYPPDIDVEWQRNGQPEPEGNYRTTPPQQDVDGTYFLYSKFSVDKASWQGGGIFQCAVMHEALHNHYTQKSISKTPGK

SEQ ID NO: 21 - protein, 337 aa, synthetic construct
ITCEPGFQYALAKDKKIGPLGAESLVTTWHDYTFDMKIQDPLVMVYCKNNRFYIGKRCEAGEERYLAKLHWRALATSVVFERVLEGRPPEEKLLDDNFEFGLCPCDSKPVVKGNFNTTLINHSAFQLVCPIGWTGTIECTLANTDTLKTTVVKRYTRTNPFPKRNGCVLHKLIGEDMHHCILGGNWTCVPGEMMKYTGGKLEKCKWCGFDFSTTEDLPTYPIGKCKVEGEVGYKFVNNETCNRGGVVISTEGKLKCSIGKTQVKVYATDKTLGPMPCRPIGVISSEGPVSKTACTFNYTETLENKYFEPRDEYFQQYMLKGKYQYWFDLKATDNKED

SEQ ID NO: 22 - protein, 336 aa, synthetic construct
FSCREGYRYALAKTKDVGPLGAESLTTTWVDYKRNLQLDDGTVRAVCSNGYFRIRPTCLIGSRFIASLHQRALPTSVTFELIPRGSAMVTEEMNDSFEFGLCPCDSKPVVKGKYNATLLNGSAFQLVCPFGWVGRVECTAVSTSTLATEVIKIYKRSTPFPYRTGCDHTTVINKDLYRCSMGGNWTCIKGEQVRYTGGVVTKCKWCDYVFKEGDGLDHYPIGKCMLKNETGYRLVDDTPCDRGGVVISKTGTLECLIGKTTVKVYSSNDKLGAMPCKPKEIISSEGPISKTACTFNYSKTLKNKYYEPRDSYFQQYMLKGEYQYWFDLDATDHHTD

SEQ ID NO: 23 - protein, 318 aa, synthetic construct
VLSGKDGLYILRSNKVWTGGSVIITDEFAVTTFIGDHTGNFKFSVKVMTTPIEMDYCIKVIDTAKFFCVMVGTPTQRDLVKPPEMLCGCGALEVQDNNSTGLISPGNVLPSKCINGWTGVVTCHCPYTDIKMKFLENTTPQKYSKNCPGTYLSDQNFHHDCKYGSQESCIDPEPTKLPPETYEDIQECFWCSYYIKDANFTPHKGPLGWCRVGENEPYYLTNRKSCVQGGVQIGSGEVTCLIGTTKIKVGNFNETAISFMPCNPIKEASRGPPSRTTCTYKYAKTLKNKIYDEKDRYWGQYMVKGEYQYWFDLEQDDH

SEQ ID NO: 24 - protein, 377 aa, synthetic construct
QTCNPEFMYALAKNTSIGSLGPESLTTRWYQLTSGFKLTDSTIEVTCVGANMRIHVVCPLVSDRYLAINHPRALPTTAWFRKIHTQHEVPRERIMSESKRRYTCPCGSKPVVRSTTQFNPISISTPSFELECPRGWTGAVECTLVSPSTLTTETIFTYRKPKPFGLENWCKYTVVEKGILYSCKFGGNSTCIKGLIVKGQREDKVRYCEWCGYKFSSPNGLPQYPLGLCEKEQSEGLRDYGDFPCCNNGTCIDKEGSVQCYIGDKKVTVKLYNASLLAPMPCKPIVYNSQGPPAPKTCTYRWASTLENKYYEPRDSYYQQYIIKSGYQYWFDLTAKDHVADWITKYFPIIIVALLGGRGTLWVLIAYELLTQYEVVG

SEQ ID NO: 25 - protein, 306 aa, synthetic construct
LYCKEGHRYAISSTKTIGPLGAEDLTTTWQAYKEGLQLDDGTIRAVCRRGNFHVRPHCGVGVRYLASLHTRALPTSVAFELLYNGPDQRIVEMNDDFEYGLCPCDSRPVVKGKYNTTLLNGSAFNLVCPIGWTGVVECTTASDTTLRTEVVRTFRRSKPFPYRDSCVVTTVVGEDLHHCSLGGNWTCIRGQRVAYSGGPVKECKWCGYTFNAPDGLPHYPIGKCMLVNETGYRYVDDTQCNREGVVIDRSGKHQCLIGKTTVNVYAVDEKLGPMPCRPKEITPSEGPVSKTACTFNYTKTLKNKYY

SEQ ID NO: 26 - protein, 336 aa, synthetic construct
LSCKPEFQYAISETNEIGPLGPTGLTTRWHAYIKGLHITDSSVDLTCVDGSFLVYKRCVKKQRYLATVHERALSTSVEFTLVAEPQDLEEVQMGDDXEFGLCPCDSTPIIRGKFNATLLNGPAFQLVCPFGWTGTIECTMVSDSTLKTQVVKRFTRYRPFPRRRYCLDQVVVGEDLYECLYGGNWTCIPGDRVLYQGGEVKDCKWCGFTFEEPSDLPHFPLGKCRLTNETGYRYVDDTTCNRDGVAITEQGTLRCKIGKVEVKVLALNKDLGPMPCKPSYITQSEGPVSKTACTFNWTETLENKYFEPRDNYFQQYMLKGKYQYWFDLEATDHHQD

SEQ ID NO: 27 - protein, 330 aa, synthetic construct
RLSCKEDYRYALAKDKKIGPLGAESLVTTWHDYTFDMKIDDGTVRAICTAGSFKVIALNVVSRRYLASLHKRALPTSVTFELLFDGTSPAIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLD

SEQ ID NO: 28 - protein, 330 aa, synthetic construct
RLSCKEDYRYALAKDKKIGPLGAESLVTTWHDYTFDMKIQDPLVMVYCKNNRFYIGALNVVSRRYLASLHKRALPTSVTFELLFDGTSPAIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLD

SEQ ID NO: 29 - protein, 330 aa, synthetic construct
RLSCKEDYRYALAKDKKIGPLGAESLVTTWHDYTFDMKIQDPLVMVYCKNNRFYIGKRCEAGEERYLAKLHWRALATSVVELLFDGTSPAIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLD

SEQ ID NO: 30 - protein, 330 aa, synthetic construct
RLSCKEDYRYALAKDKKIGPLGAESLVTTWHDYTFDMKIQDPLVMVYCKNNRFYIGKRCEAGEERYLAKLHWRALATSVVFERVLEGRPPEEKLLDDNFEFGLCPCDSKPVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLD

SEQ ID NO: 31 - protein, 330 aa, synthetic construct
RLSCKEDYRYALAKTKDVGPLGAESLTTTWVDYKRNLQLDDGTVRAICTAGSFKVIALNVVSRRYLASLHKRALPTSVTFELLFDGTSPAIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLD

SEQ ID NO: 32 - protein, 330 aa, synthetic construct
RLSCKEDYRYALAKTKDVGPLGAESLTTTWVDYKRNLQLDDGTVRAVCSNGYFRIRALNVVSRRYLASLHKRALPTSVTFELLFDGTSPAIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLD

SEQ ID NO: 33 - protein, 330 aa, synthetic construct
RLSCKEDYRYALAKTKDVGPLGAESLTTTWVDYKRNLQLDDGTVRAVCSNGYFRIRPTCLIGSRFIASLHQRALPTSVTFELLFDGTSPAIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLD

SEQ ID NO: 34 - protein, 330 aa, synthetic construct
RLSCKEDYRYALAKTKDVGPLGAESLTTTWVDYKRNLQLDDGTVRAVCSNGYFRIRPTCLIGSRFIASLHQRALPTSVTFELIPRGSAMVTEEMNDSFEFGLCPCDSKPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLD

SEQ ID NO: 35 - protein, 330 aa, synthetic construct
RLSCKEDYRYVLSGKDGLYILRSNKVWTGGSVIITDEFADDGTVRAICTAGSFKVIALNVVSRRYLASLHKRALPTSVTFELLFDGTSPAIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLD

SEQ ID NO: 36 - protein, 330 aa, synthetic construct
RLSCKEDYRYALAKNTSIGSLGPESLTTRWYQLTSGFKLDDGTVRAICTAGSFKVIALNVVSRRYLASLHKRALPTSVTFELLFDGTSPAIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLD

SEQ ID NO: 37 - protein, 330 aa, synthetic construct
RLSCKEDYRYALAKNTSIGSLGPESLTTRWYQLTSGFKLTDSTIEVTCVGANMRIHALNVVSRRYLASLHKRALPTSVTFELLFDGTSPAIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLD

SEQ ID NO: 38 - protein, 332 aa, synthetic construct
RLSCKEDYRYALAKNTSIGSLGPESLTTRWYQLTSGFKLTDSTIEVTCVGANMRIHVVCPLVSDRYLAINHPRALPTTAWFRKIHTQHEVPRERIMSESKRRYTCPCGSKPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLD

SEQ ID NO: 39 - protein, 330 aa, synthetic construct
RLSCKEDYRYAISSTKTIGPLGAEDLTTTWQAYKEGLQLDDGTVRAICTAGSFKVIALNVVSRRYLASLHKRALPTSVTFELLFDGTSPAIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLD

SEQ ID NO: 40 - protein, 330 aa, synthetic construct
RLSCKEDYRYAISSTKTIGPLGAEDLTTTWQAYKEGLQLDDGTIRAVCRRGNFHVRALNVVSRRYLASLHKRALPTSVTFELLFDGTSPAIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLD

SEQ ID NO: 41 - protein, 330 aa, synthetic construct
RLSCKEDYRYAISSTKTIGPLGAEDLTTTWQAYKEGLQLDDGTIRAVCRRGNFHVRPHCGVGVRYLASLHTRALPTSVAFELLFDGTSPAIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLD

SEQ ID NO: 42 - protein, 330 aa, synthetic construct
RLSCKEDYRYAISSTKTIGPLGAEDLTTTWQAYKEGLQLDDGTIRAVCRRGNFHVRPHCGVGVRYLASLHTRALPTSVAFELLYNGPDQRIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLD

SEQ ID NO: 43 - protein, 330 aa, synthetic construct
RLSCKEDYRYAISSTKTIGPLGAEDLTTTWQAYKEGLQLDDGTIRAVCRRGNFHVRPHCGVGVRYLASLHTRALPTSVAFELLYNGPDQRIVEMNDDFEYGLCPCDSRPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLD

SEQ ID NO: 44 - protein, 330 aa, synthetic construct
RLSCKEDYRYAISETNEIGPLGPTGLTTRWHAYIKGLHIDDGTVRAICTAGSFKVIALNVVSRRYLASLHKRALPTSVTFELLFDGTSPAIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLD

SEQ ID NO: 45 - protein, 330 aa, synthetic construct
RLSCKEDYRYAISETNEIGPLGPTGLTTRWHAYIKGLHITDSSVDLTCVDGSFLVYALNVVSRRYLASLHKRALPTSVTFELLFDGTSPAIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLD

SEQ ID NO: 46 - protein, 330 aa, synthetic construct
RLSCKEDYRYAISETNEIGPLGPTGLTTRWHAYIKGLHITDSSVDLTCVDGSFLVYKRCVKKQRYLATVHERALSTSVEFELLFDGTSPAIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLD

SEQ ID NO: 47 - protein, 333 aa, synthetic construct
RLSCKEDYRYAISETNEIGPLGPTGLTTRWHAYIKGLHITDSSVDLTCVDGSFLVYKRCVKKQRYLATVHERALSTSVEFTLVAEPQDLEIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLDGGS

SEQ ID NO: 48 - protein, 330 aa, synthetic construct
RLSCKEDYRYAISETNEIGPLGPTGLTTRWHAYIKGLHITDSSVDLTCVDGSFLVYKRCVKKQRYLATVHERALSTSVEFTLVAEPQDLEEVQMGDDFEFGLCPCDSTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLD

SEQ ID NO: 49 - protein, 562 aa, synthetic construct
RLSCKEDYRYVLSGKDGLYILRSNKVWTGGSVIITDEFADDGTVRAICTAGSFKVIALNVVSRRYLASLHKRALPTSVTFELLFDGTSPAIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLDGGSTKTKPPCPICPACESPGPSVFIFPPKPKDTLMISRTPQVTCVVVDVSQENPEVQFSWYVDGVEVHTAQTRPKEEQFNSTYRVVSVLPIQHQDWLNGKEFKCKVNNKDLPAPITRIISKAKGQTREPQVYTLPPHAEELSRSKVSITCLVIGFYPPDIDVEWQRNGQPEPEGNYRTTPPQQDVDGTYFLYSKFSVDKASWQGGGIFQCAVMHEALHNHYTQKSISKTPGK

SEQ ID NO: 50 - protein, 564 aa, synthetic construct
RLSCKEDYRYALAKNTSIGSLGPESLTTRWYQLTSGFKLTDSTIEVTCVGANMRIHVVCPLVSDRYLAINHPRALPTTAWFRKIHTQHEVPRERIMSESKRRYTCPCGSKPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLDGGSTKTKPPCPICPACESPGPSVFIFPPKPKDTLMISRTPQVTCVVVDVSQENPEVQFSWYVDGVEVHTAQTRPKEEQFNSTYRVVSVLPIQHQDWLNGKEFKCKVNNKDLPAPITRIISKAKGQTREPQVYTLPPHAEELSRSKVSITCLVIGFYPPDIDVEWQRNGQPEPEGNYRTTPPQQDVDGTYFLYSKFSVDKASWQGGGIFQCAVMHEALHNHYTQKSISKTPGK

SEQ ID NO: 51 - protein, 562 aa, synthetic construct
RLSCKEDYRYALAKNTSIGSLGPESLTTRWYQLTSGFKLTDSTIEVTCVGANMRIHALNVVSRRYLASLHKRALPTSVTFELLFDGTSPAIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLDGGSTKTKPPCPICPACESPGPSVFIFPPKPKDTLMISRTPQVTCVVVDVSQENPEVQFSWYVDGVEVHTAQTRPKEEQFNSTYRVVSVLPIQHQDWLNGKEFKCKVNNKDLPAPITRIISKAKGQTREPQVYTLPPHAEELSRSKVSITCLVIGFYPPDIDVEWQRNGQPEPEGNYRTTPPQQDVDGTYFLYSKFSVDKASWQGGGIFQCAVMHEALHNHYTQKSISKTPGK

SEQ ID NO: 52 - protein, 562 aa, synthetic construct
RLSCKEDYRYALAKNTSIGSLGPESLTTRWYQLTSGFKLDDGTVRAICTAGSFKVIALNVVSRRYLASLHKRALPTSVTFELLFDGTSPAIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLDGGSTKTKPPCPICPACESPGPSVFIFPPKPKDTLMISRTPQVTCVVVDVSQENPEVQFSWYVDGVEVHTAQTRPKEEQFNSTYRVVSVLPIQHQDWLNGKEFKCKVNNKDLPAPITRIISKAKGQTREPQVYTLPPHAEELSRSKVSITCLVIGFYPPDIDVEWQRNGQPEPEGNYRTTPPQQDVDGTYFLYSKFSVDKASWQGGGIFQCAVMHEALHNHYTQKSISKTPGK

SEQ ID NO: 53 - protein, 562 aa, synthetic construct
RLSCKEDYRYAISETNEIGPLGPTGLTTRWHAYIKGLHITDSSVDLTCVDGSFLVYKRCVKKQRYLATVHERALSTSVEFTLVAEPQDLEEVQMGDDFEFGLCPCDSTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLDGGSTKTKPPCPICPACESPGPSVFIFPPKPKDTLMISRTPQVTCVVVDVSQENPEVQFSWYVDGVEVHTAQTRPKEEQFNSTYRVVSVLPIQHQDWLNGKEFKCKVNNKDLPAPITRIISKAKGQTREPQVYTLPPHAEELSRSKVSITCLVIGFYPPDIDVEWQRNGQPEPEGNYRTTPPQQDVDGTYFLYSKFSVDKASWQGGGIFQCAVMHEALHNHYTQKSISKTPGK

SEQ ID NO: 54 - protein, 562 aa, synthetic construct
RLSCKEDYRYAISETNEIGPLGPTGLTTRWHAYIKGLHITDSSVDLTCVDGSFLVYKRCVKKQRYLATVHERALSTSVEFTLVAEPQDLEIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLDGGSTKTKPPCPICPACESPGPSVFIFPPKPKDTLMISRTPQVTCVVVDVSQENPEVQFSWYVDGVEVHTAQTRPKEEQFNSTYRVVSVLPIQHQDWLNGKEFKCKVNNKDLPAPITRIISKAKGQTREPQVYTLPPHAEELSRSKVSITCLVIGFYPPDIDVEWQRNGQPEPEGNYRTTPPQQDVDGTYFLYSKFSVDKASWQGGGIFQCAVMHEALHNHYTQKSISKTPGK

SEQ ID NO: 55 - protein, 562 aa, synthetic construct
RLSCKEDYRYAISETNEIGPLGPTGLTTRWHAYIKGLHITDSSVDLTCVDGSFLVYKRCVKKQRYLATVHERALSTSVEFELLFDGTSPAIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLDGGSTKTKPPCPICPACESPGPSVFIFPPKPKDTLMISRTPQVTCVVVDVSQENPEVQFSWYVDGVEVHTAQTRPKEEQFNSTYRVVSVLPIQHQDWLNGKEFKCKVNNKDLPAPITRIISKAKGQTREPQVYTLPPHAEELSRSKVSITCLVIGFYPPDIDVEWQRNGQPEPEGNYRTTPPQQDVDGTYFLYSKFSVDKASWQGGGIFQCAVMHEALHNHYTQKSISKTPGK

SEQ ID NO: 56 - protein, 562 aa, synthetic construct
RLSCKEDYRYAISETNEIGPLGPTGLTTRWHAYIKGLHITDSSVDLTCVDGSFLVYALNVVSRRYLASLHKRALPTSVTFELLFDGTSPAIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLDGGSTKTKPPCPICPACESPGPSVFIFPPKPKDTLMISRTPQVTCVVVDVSQENPEVQFSWYVDGVEVHTAQTRPKEEQFNSTYRVVSVLPIQHQDWLNGKEFKCKVNNKDLPAPITRIISKAKGQTREPQVYTLPPHAEELSRSKVSITCLVIGFYPPDIDVEWQRNGQPEPEGNYRTTPPQQDVDGTYFLYSKFSVDKASWQGGGIFQCAVMHEALHNHYTQKSISKTPGK

SEQ ID NO: 57 - protein, 562 aa, synthetic construct
RLSCKEDYRYAISETNEIGPLGPTGLTTRWHAYIKGLHIDDGTVRAICTAGSFKVIALNVVSRRYLASLHKRALPTSVTFELLFDGTSPAIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLDGGSTKTKPPCPICPACESPGPSVFIFPPKPKDTLMISRTPQVTCVVVDVSQENPEVQFSWYVDGVEVHTAQTRPKEEQFNSTYRVVSVLPIQHQDWLNGKEFKCKVNNKDLPAPITRIISKAKGQTREPQVYTLPPHAEELSRSKVSITCLVIGFYPPDIDVEWQRNGQPEPEGNYRTTPPQQDVDGTYFLYSKFSVDKASWQGGGIFQCAVMHEALHNHYTQKSISKTPGK

SEQ ID NO: 58 - protein, 562 aa, synthetic construct
RLSCKEDYRYALAKDKKIGPLGAESLVTTWHDYTFDMKIQDPLVMVYCKNNRFYIGKRCEAGEERYLAKLHWRALATSVVFERVLEGRPPEEKLLDDNFEFGLCPCDSKPVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLDGGSTKTKPPCPICPACESPGPSVFIFPPKPKDTLMISRTPQVTCVVVDVSQENPEVQFSWYVDGVEVHTAQTRPKEEQFNSTYRVVSVLPIQHQDWLNGKEFKCKVNNKDLPAPITRIISKAKGQTREPQVYTLPPHAEELSRSKVSITCLVIGFYPPDIDVEWQRNGQPEPEGNYRTTPPQQDVDGTYFLYSKFSVDKASWQGGGIFQCAVMHEALHNHYTQKSISKTPGK

SEQ ID NO: 59 - protein, 562 aa, synthetic construct
RLSCKEDYRYALAKDKKIGPLGAESLVTTWHDYTFDMKIQDPLVMVYCKNNRFYIGKRCEAGEERYLAKLHWRALATSVVELLFDGTSPAIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLDGGSTKTKPPCPICPACESPGPSVFIFPPKPKDTLMISRTPQVTCVVVDVSQENPEVQFSWYVDGVEVHTAQTRPKEEQFNSTYRVVSVLPIQHQDWLNGKEFKCKVNNKDLPAPITRIISKAKGQTREPQVYTLPPHAEELSRSKVSITCLVIGFYPPDIDVEWQRNGQPEPEGNYRTTPPQQDVDGTYFLYSKFSVDKASWQGGGIFQCAVMHEALHNHYTQKSISKTPGK

SEQ ID NO: 60 - protein, 562 aa, synthetic construct
RLSCKEDYRYALAKDKKIGPLGAESLVTTWHDYTFDMKIQDPLVMVYCKNNRFYIGALNVVSRRYLASLHKRALPTSVTFELLFDGTSPAIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLDGGSTKTKPPCPICPACESPGPSVFIFPPKPKDTLMISRTPQVTCVVVDVSQENPEVQFSWYVDGVEVHTAQTRPKEEQFNSTYRVVSVLPIQHQDWLNGKEFKCKVNNKDLPAPITRIISKAKGQTREPQVYTLPPHAEELSRSKVSITCLVIGFYPPDIDVEWQRNGQPEPEGNYRTTPPQQDVDGTYFLYSKFSVDKASWQGGGIFQCAVMHEALHNHYTQKSISKTPGK

SEQ ID NO: 61 - protein, 562 aa, synthetic construct
RLSCKEDYRYALAKDKKIGPLGAESLVTTWHDYTFDMKIDDGTVRAICTAGSFKVIALNVVSRRYLASLHKRALPTSVTFELLFDGTSPAIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLDGGSTKTKPPCPICPACESPGPSVFIFPPKPKDTLMISRTPQVTCVVVDVSQENPEVQFSWYVDGVEVHTAQTRPKEEQFNSTYRVVSVLPIQHQDWLNGKEFKCKVNNKDLPAPITRIISKAKGQTREPQVYTLPPHAEELSRSKVSITCLVIGFYPPDIDVEWQRNGQPEPEGNYRTTPPQQDVDGTYFLYSKFSVDKASWQGGGIFQCAVMHEALHNHYTQKSISKTPGK

SEQ ID NO: 62 - protein, 562 aa, synthetic construct
RLSCKEDYRYALAKTKDVGPLGAESLTTTWVDYKRNLQLDDGTVRAICTAGSFKVIALNVVSRRYLASLHKRALPTSVTFELLFDGTSPAIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLDGGSTKTKPPCPICPACESPGPSVFIFPPKPKDTLMISRTPQVTCVVVDVSQENPEVQFSWYVDGVEVHTAQTRPKEEQFNSTYRVVSVLPIQHQDWLNGKEFKCKVNNKDLPAPITRIISKAKGQTREPQVYTLPPHAEELSRSKVSITCLVIGFYPPDIDVEWQRNGQPEPEGNYRTTPPQQDVDGTYFLYSKFSVDKASWQGGGIFQCAVMHEALHNHYTQKSISKTPGK

SEQ ID NO: 63 - protein, 562 aa, synthetic construct
RLSCKEDYRYALAKTKDVGPLGAESLTTTWVDYKRNLQLDDGTVRAVCSNGYFRIRALNVVSRRYLASLHKRALPTSVTFELLFDGTSPAIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLDGGSTKTKPPCPICPACESPGPSVFIFPPKPKDTLMISRTPQVTCVVVDVSQENPEVQFSWYVDGVEVHTAQTRPKEEQFNSTYRVVSVLPIQHQDWLNGKEFKCKVNNKDLPAPITRIISKAKGQTREPQVYTLPPHAEELSRSKVSITCLVIGFYPPDIDVEWQRNGQPEPEGNYRTTPPQQDVDGTYFLYSKFSVDKASWQGGGIFQCAVMHEALHNHYTQKSISKTPGK

SEQ ID NO: 64 - protein, 562 aa, synthetic construct
RLSCKEDYRYALAKTKDVGPLGAESLTTTWVDYKRNLQLDDGTVRAVCSNGYFRIRPTCLIGSRFIASLHQRALPTSVTFELLFDGTSPAIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLDGGSTKTKPPCPICPACESPGPSVFIFPPKPKDTLMISRTPQVTCVVVDVSQENPEVQFSWYVDGVEVHTAQTRPKEEQFNSTYRVVSVLPIQHQDWLNGKEFKCKVNNKDLPAPITRIISKAKGQTREPQVYTLPPHAEELSRSKVSITCLVIGFYPPDIDVEWQRNGQPEPEGNYRTTPPQQDVDGTYFLYSKFSVDKASWQGGGIFQCAVMHEALHNHYTQKSISKTPGK

SEQ ID NO: 65 - protein, 562 aa, synthetic construct
RLSCKEDYRYALAKTKDVGPLGAESLTTTWVDYKRNLQLDDGTVRAVCSNGYFRIRPTCLIGSRFIASLHQRALPTSVTFELIPRGSAMVTEEMNDSFEFGLCPCDSKPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLDGGSTKTKPPCPICPACESPGPSVFIFPPKPKDTLMISRTPQVTCVVVDVSQENPEVQFSWYVDGVEVHTAQTRPKEEQFNSTYRVVSVLPIQHQDWLNGKEFKCKVNNKDLPAPITRIISKAKGQTREPQVYTLPPHAEELSRSKVSITCLVIGFYPPDIDVEWQRNGQPEPEGNYRTTPPQQDVDGTYFLYSKFSVDKASWQGGGIFQCAVMHEALHNHYTQKSISKTPGK

SEQ ID NO: 66 - protein, 562 aa, synthetic construct
RLSCKEDYRYAISSTKTIGPLGAEDLTTTWQAYKEGLQLDDGTIRAVCRRGNFHVRPHCGVGVRYLASLHTRALPTSVAFELLYNGPDQRIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLDGGSTKTKPPCPICPACESPGPSVFIFPPKPKDTLMISRTPQVTCVVVDVSQENPEVQFSWYVDGVEVHTAQTRPKEEQFNSTYRVVSVLPIQHQDWLNGKEFKCKVNNKDLPAPITRIISKAKGQTREPQVYTLPPHAEELSRSKVSITCLVIGFYPPDIDVEWQRNGQPEPEGNYRTTPPQQDVDGTYFLYSKFSVDKASWQGGGIFQCAVMHEALHNHYTQKSISKTPGK

SEQ ID NO: 67 - protein, 562 aa, synthetic construct
RLSCKEDYRYAISSTKTIGPLGAEDLTTTWQAYKEGLQLDDGTIRAVCRRGNFHVRPHCGVGVRYLASLHTRALPTSVAFELLFDGTSPAIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLDGGSTKTKPPCPICPACESPGPSVFIFPPKPKDTLMISRTPQVTCVVVDVSQENPEVQFSWYVDGVEVHTAQTRPKEEQFNSTYRVVSVLPIQHQDWLNGKEFKCKVNNKDLPAPITRIISKAKGQTREPQVYTLPPHAEELSRSKVSITCLVIGFYPPDIDVEWQRNGQPEPEGNYRTTPPQQDVDGTYFLYSKFSVDKASWQGGGIFQCAVMHEALHNHYTQKSISKTPGK

SEQ ID NO: 68 - protein, 562 aa, synthetic construct
RLSCKEDYRYAISSTKTIGPLGAEDLTTTWQAYKEGLQLDDGTIRAVCRRGNFHVRPHCGVGVRYLASLHTRALPTSVAFELLYNGPDQRIVEMNDDFEYGLCPCDSRPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLDGGSTKTKPPCPICPACESPGPSVFIFPPKPKDTLMISRTPQVTCVVVDVSQENPEVQFSWYVDGVEVHTAQTRPKEEQFNSTYRVVSVLPIQHQDWLNGKEFKCKVNNKDLPAPITRIISKAKGQTREPQVYTLPPHAEELSRSKVSITCLVIGFYPPDIDVEWQRNGQPEPEGNYRTTPPQQDVDGTYFLYSKFSVDKASWQGGGIFQCAVMHEALHNHYTQKSISKTPGK

SEQ ID NO: 69 - protein, 562 aa, synthetic construct
RLSCKEDYRYAISSTKTIGPLGAEDLTTTWQAYKEGLQLDDGTIRAVCRRGNFHVRALNVVSRRYLASLHKRALPTSVTFELLFDGTSPAIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLDGGSTKTKPPCPICPACESPGPSVFIFPPKPKDTLMISRTPQVTCVVVDVSQENPEVQFSWYVDGVEVHTAQTRPKEEQFNSTYRVVSVLPIQHQDWLNGKEFKCKVNNKDLPAPITRIISKAKGQTREPQVYTLPPHAEELSRSKVSITCLVIGFYPPDIDVEWQRNGQPEPEGNYRTTPPQQDVDGTYFLYSKFSVDKASWQGGGIFQCAVMHEALHNHYTQKSISKTPGK

SEQ ID NO: 70 - protein, 562 aa, synthetic construct
RLSCKEDYRYAISSTKTIGPLGAEDLTTTWQAYKEGLQLDDGTVRAICTAGSFKVIALNVVSRRYLASLHKRALPTSVTFELLFDGTSPAIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLDGGSTKTKPPCPICPACESPGPSVFIFPPKPKDTLMISRTPQVTCVVVDVSQENPEVQFSWYVDGVEVHTAQTRPKEEQFNSTYRVVSVLPIQHQDWLNGKEFKCKVNNKDLPAPITRIISKAKGQTREPQVYTLPPHAEELSRSKVSITCLVIGFYPPDIDVEWQRNGQPEPEGNYRTTPPQQDVDGTYFLYSKFSVDKASWQGGGIFQCAVMHEALHNHYTQKSISKTPGK

SEQ ID NO: 71 - protein, 330 aa, synthetic construct
RLSCKEDYRYAISKTKTIGPLAARDLTTTWQAYKEGLQLDDGTVRAICTAGSFKVIALNVVSRRYLASLHKRALPTSVTFELLFDGTSPAIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLD

SEQ ID NO: 72 - protein, 330 aa, synthetic construct
RLSCKEDYRYAISKTKTIGPLAARDLTTTWQAYKEGLQLDDGTIRAVCRRGNFHVRPHCGVGVRYLASLHTRALPTSVAFELLYNGPDQRIVEMNDDFEYGLCPCDSRPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLD

SEQ ID NO: 73 - protein, 330 aa, synthetic construct
RLSCKEDYRYAISKTKTIGPLAARDLTTTWQAYKEGLQLDDGTIRAVCRRGNFHVRALNVVSRRYLASLHKRALPTSVTFELLFDGTSPAIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLD

SEQ ID NO: 74 - protein, 330 aa, synthetic construct
RLSCKEDYRYALAKTKDVGPLAARDLTTTWVDYKRNLQLDDGTVRAVCSNGYFRIRALNVVSRRYLASLHKRALPTSVTFELLFDGTSPAIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLD

SEQ ID NO: 75 - protein, 562 aa, synthetic construct
RLSCKEDYRYAISKTKTIGPLAARDLTTTWQAYKEGLQLDDGTVRAICTAGSFKVIALNVVSRRYLASLHKRALPTSVTFELLFDGTSPAIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLDGGSTKTKPPCPICPACESPGPSVFIFPPKPKDTLMISRTPQVTCVVVDVSQENPEVQFSWYVDGVEVHTAQTRPKEEQFNSTYRVVSVLPIQHQDWLNGKEFKCKVNNKDLPAPITRIISKAKGQTREPQVYTLPPHAEELSRSKVSITCLVIGFYPPDIDVEWQRNGQPEPEGNYRTTPPQQDVDGTYFLYSKFSVDKASWQGGGIFQCAVMHEALHNHYTQKSISKTPGK

SEQ ID NO: 76 - protein, 562 aa, synthetic construct
RLSCKEDYRYAISKTKTIGPLAARDLTTTWQAYKEGLQLDDGTIRAVCRRGNFHVRPHCGVGVRYLASLHTRALPTSVAFELLYNGPDQRIVEMNDDFEYGLCPCDSRPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLDGGSTKTKPPCPICPACESPGPSVFIFPPKPKDTLMISRTPQVTCVVVDVSQENPEVQFSWYVDGVEVHTAQTRPKEEQFNSTYRVVSVLPIQHQDWLNGKEFKCKVNNKDLPAPITRIISKAKGQTREPQVYTLPPHAEELSRSKVSITCLVIGFYPPDIDVEWQRNGQPEPEGNYRTTPPQQDVDGTYFLYSKFSVDKASWQGGGIFQCAVMHEALHNHYTQKSISKTPGK

SEQ ID NO: 77 - protein, 562 aa, synthetic construct
RLSCKEDYRYAISKTKTIGPLAARDLTTTWQAYKEGLQLDDGTIRAVCRRGNFHVRALNVVSRRYLASLHKRALPTSVTFELLFDGTSPAIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLDGGSTKTKPPCPICPACESPGPSVFIFPPKPKDTLMISRTPQVTCVVVDVSQENPEVQFSWYVDGVEVHTAQTRPKEEQFNSTYRVVSVLPIQHQDWLNGKEFKCKVNNKDLPAPITRIISKAKGQTREPQVYTLPPHAEELSRSKVSITCLVIGFYPPDIDVEWQRNGQPEPEGNYRTTPPQQDVDGTYFLYSKFSVDKASWQGGGIFQCAVMHEALHNHYTQKSISKTPGK

SEQ ID NO: 78 - protein, 562 aa, synthetic construct
RLSCKEDYRYALAKTKDVGPLAARDLTTTWVDYKRNLQLDDGTVRAVCSNGYFRIRALNVVSRRYLASLHKRALPTSVTFELLFDGTSPAIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLDGGSTKTKPPCPICPACESPGPSVFIFPPKPKDTLMISRTPQVTCVVVDVSQENPEVQFSWYVDGVEVHTAQTRPKEEQFNSTYRVVSVLPIQHQDWLNGKEFKCKVNNKDLPAPITRIISKAKGQTREPQVYTLPPHAEELSRSKVSITCLVIGFYPPDIDVEWQRNGQPEPEGNYRTTPPQQDVDGTYFLYSKFSVDKASWQGGGIFQCAVMHEALHNHYTQKSISKTPGK

SEQ ID NO: 79 - protein, 330 aa, synthetic construct
RLSCKEDYRPAISSTNEIGPLGAEGLTTTWKEYNHGLQLDDGTVRAICTAGSFKVIALNVVSRRYLASLHKRALPTSVTFELLFDGTSPAIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLD

SEQ ID NO: 80 - protein, 330 aa, synthetic construct
RLSCKEDYRAAISSTNEIGPLGAEGLTTTWKEYNHGLQLDDGTVRAICTAGSFKVIALNVVSRRYLASLHKRALPTSVTFELLFDGTSPAIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLD

SEQ ID NO: 81 - protein, 330 aa, synthetic construct
RLSCKEDYRYAISSTNEIGPLGAEGLTTTWKEYNHGLQLDAGTVRAICTAGSFKVIALNVVSRRYLASLHKRALPTSVTFELLFDGTSPAIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLD

SEQ ID NO: 82 - protein, 330 aa, synthetic construct
RLSCKEDYRYAISSTNEIGPLGAEGLTTTWKEYNHGLQLDNGTVRAICTAGSFKVIALNVVSRRYLASLHKRALPTSVTFELLFDGTSPAIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLD

SEQ ID NO: 83 - protein, 330 aa, synthetic construct
RLSCKEDYRYAISSTNEIGPLGAEGLTTTWKEYNHGLQLDEGTVRAICTAGSFKVIALNVVSRRYLASLHKRALPTSVTFELLFDGTSPAIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLD

SEQ ID NO: 84 - protein, 330 aa, synthetic construct
RLSCKEDYRYAISSTNEIGPLGAEGLTTTWKEYNHGLQLDDGTVRAICTAGSFKVIALNVVSRAYLASLHKRALPTSVTFELLFDGTSPAIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLD

SEQ ID NO: 85 - protein, 330 aa, synthetic construct
RLSCKEDYRYAISSTNEIGPLGAEGLTTTWKEYNHGLQLDDGTVRAICTAGSFKVIALNVVSRSYLASLHKRALPTSVTFELLFDGTSPAIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLD

SEQ ID NO: 86 - protein, 330 aa, synthetic construct
RLSCKEDYRYAISSTNEIGPLGAEGLTTTWKEYNHGLQLDDGTVRAICTAGSFKVIALNVVSREYLASLHKRALPTSVTFELLFDGTSPAIEEMGDDFGFGLCPFDTTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLD

SEQ ID NO: 87 - DNA, 1053 bp, synthetic construct
  source 1..1053: mol_type = other DNA
attctgagggggcaggtcgtccagggcattatttggctgctgctggtcacaggggcacaggggagactgagttgtaaagaggattatcgccccgctatctccagcaccaacgagatcggccctctgggcgccgagggcctgaccacaacatggaaggagtacaatcacggcctgcaactggacgatggcaccgtgagggctatctgtaccgctggctcctttaaggtaatcgctctgaacgtggtgtctcggaggtatctggcttccctgcataagagagccctgccaacatctgtgaccttcgagctgctgttcgatggcacctcccctgccatcgaggagatgggcgacgattttggcttcggcctgtgccccttcgacaccacaccagtggtgaagggcaagtataacaccacactgctgaatggctccgctttctacctggtgtgccctatcggctggacaggcgtgatcgagtgtacagccgtgtctccaaccacactgagaaccgaggtggtgaaaaccttcaagagggagaagccatttccccatagggtgggctgcgtgaccacagtggtggagaaggaggatctgttctactgcaagtggggcggcaattggacctgcgtgaagggcaatcctgtgacctacatgggcggccaggtgaagcagtgtcggtggtgcggcttcgactttaaggagcctgatggcctgccacactaccccatcggcaagtgcatcctggccaatgagacaggctatagagtggtggattccacagactgcaatagagacggcgtggtaatctctaccgagggcgagcacgagtgcctgatcggcaatacaacagtgaaggtgcacgccctggatggcaggctgggacccatgccatgtcggcctaaggagatcgtgtcctctgctggcccagtgagaaagacctcttgtaccttcaattatacaaagaccctgagaaacaagtattacgagccacgggactcttatttccagcagtatatgctgaagggggagtatcagtattggtttgacctggat

SEQ ID NO: 88 - DNA, 1053 bp, synthetic construct
  source 1..1053: mol_type = other DNA
attctgagggggcaggtcgtccagggcattatttggctgctgctggtcacaggggcacaggggagactgagttgtaaagaggattatcgcgccgctatctccagcaccaacgagatcggccctctgggcgccgagggcctgaccacaacatggaaggagtacaatcacggcctgcaactggacgatggcaccgtgagggctatctgtaccgctggctcctttaaggtaatcgctctgaacgtggtgtctcggaggtatctggcttccctgcataagagagccctgccaacatctgtgaccttcgagctgctgttcgatggcacctcccctgccatcgaggagatgggcgacgattttggcttcggcctgtgccccttcgacaccacaccagtggtgaagggcaagtataacaccacactgctgaatggctccgctttctacctggtgtgccctatcggctggacaggcgtgatcgagtgtacagccgtgtctccaaccacactgagaaccgaggtggtgaaaaccttcaagagggagaagccatttccccatagggtgggctgcgtgaccacagtggtggagaaggaggatctgttctactgcaagtggggcggcaattggacctgcgtgaagggcaatcctgtgacctacatgggcggccaggtgaagcagtgtcggtggtgcggcttcgactttaaggagcctgatggcctgccacactaccccatcggcaagtgcatcctggccaatgagacaggctatagagtggtggattccacagactgcaatagagacggcgtggtaatctctaccgagggcgagcacgagtgcctgatcggcaatacaacagtgaaggtgcacgccctggatggcaggctgggacccatgccatgtcggcctaaggagatcgtgtcctctgctggcccagtgagaaagacctcttgtaccttcaattatacaaagaccctgagaaacaagtattacgagccacgggactcttatttccagcagtatatgctgaagggggagtatcagtattggtttgacctggat

SEQ ID NO: 89 - DNA, 1053 bp, synthetic construct
  source 1..1053: mol_type = other DNA
attctgagggggcaggtcgtccagggcattatttggctgctgctggtcacaggggcacaggggagactgagttgtaaagaggattatcgctatgctatctccagcaccaacgagatcggccctctgggcgccgagggcctgaccacaacatggaaggagtacaatcacggcctgcaactggacgccggcaccgtgagggctatctgtaccgctggctcctttaaggtaatcgctctgaacgtggtgtctcggaggtatctggcttccctgcataagagagccctgccaacatctgtgaccttcgagctgctgttcgatggcacctcccctgccatcgaggagatgggcgacgattttggcttcggcctgtgccccttcgacaccacaccagtggtgaagggcaagtataacaccacactgctgaatggctccgctttctacctggtgtgccctatcggctggacaggcgtgatcgagtgtacagccgtgtctccaaccacactgagaaccgaggtggtgaaaaccttcaagagggagaagccatttccccatagggtgggctgcgtgaccacagtggtggagaaggaggatctgttctactgcaagtggggcggcaattggacctgcgtgaagggcaatcctgtgacctacatgggcggccaggtgaagcagtgtcggtggtgcggcttcgactttaaggagcctgatggcctgccacactaccccatcggcaagtgcatcctggccaatgagacaggctatagagtggtggattccacagactgcaatagagacggcgtggtaatctctaccgagggcgagcacgagtgcctgatcggcaatacaacagtgaaggtgcacgccctggatggcaggctgggacccatgccatgtcggcctaaggagatcgtgtcctctgctggcccagtgagaaagacctcttgtaccttcaattatacaaagaccctgagaaacaagtattacgagccacgggactcttatttccagcagtatatgctgaagggggagtatcagtattggtttgacctggat

SEQ ID NO: 90 - DNA, 1053 bp, synthetic construct
  source 1..1053: mol_type = other DNA
attctgagggggcaggtcgtccagggcattatttggctgctgctggtcacaggggcacaggggagactgagttgtaaagaggattatcgctatgctatctccagcaccaacgagatcggccctctgggcgccgagggcctgaccacaacatggaaggagtacaatcacggcctgcaactggacaacggcaccgtgagggctatctgtaccgctggctcctttaaggtaatcgctctgaacgtggtgtctcggaggtatctggcttccctgcataagagagccctgccaacatctgtgaccttcgagctgctgttcgatggcacctcccctgccatcgaggagatgggcgacgattttggcttcggcctgtgccccttcgacaccacaccagtggtgaagggcaagtataacaccacactgctgaatggctccgctttctacctggtgtgccctatcggctggacaggcgtgatcgagtgtacagccgtgtctccaaccacactgagaaccgaggtggtgaaaaccttcaagagggagaagccatttccccatagggtgggctgcgtgaccacagtggtggagaaggaggatctgttctactgcaagtggggcggcaattggacctgcgtgaagggcaatcctgtgacctacatgggcggccaggtgaagcagtgtcggtggtgcggcttcgactttaaggagcctgatggcctgccacactaccccatcggcaagtgcatcctggccaatgagacaggctatagagtggtggattccacagactgcaatagagacggcgtggtaatctctaccgagggcgagcacgagtgcctgatcggcaatacaacagtgaaggtgcacgccctggatggcaggctgggacccatgccatgtcggcctaaggagatcgtgtcctctgctggcccagtgagaaagacctcttgtaccttcaattatacaaagaccctgagaaacaagtattacgagccacgggactcttatttccagcagtatatgctgaagggggagtatcagtattggtttgacctggat

SEQ ID NO: 91 - DNA, 1053 bp, synthetic construct
  source 1..1053: mol_type = other DNA
attctgagggggcaggtcgtccagggcattatttggctgctgctggtcacaggggcacaggggagactgagttgtaaagaggattatcgctatgctatctccagcaccaacgagatcggccctctgggcgccgagggcctgaccacaacatggaaggagtacaatcacggcctgcaactggacgagggcaccgtgagggctatctgtaccgctggctcctttaaggtaatcgctctgaacgtggtgtctcggaggtatctggcttccctgcataagagagccctgccaacatctgtgaccttcgagctgctgttcgatggcacctcccctgccatcgaggagatgggcgacgattttggcttcggcctgtgccccttcgacaccacaccagtggtgaagggcaagtataacaccacactgctgaatggctccgctttctacctggtgtgccctatcggctggacaggcgtgatcgagtgtacagccgtgtctccaaccacactgagaaccgaggtggtgaaaaccttcaagagggagaagccatttccccatagggtgggctgcgtgaccacagtggtggagaaggaggatctgttctactgcaagtggggcggcaattggacctgcgtgaagggcaatcctgtgacctacatgggcggccaggtgaagcagtgtcggtggtgcggcttcgactttaaggagcctgatggcctgccacactaccccatcggcaagtgcatcctggccaatgagacaggctatagagtggtggattccacagactgcaatagagacggcgtggtaatctctaccgagggcgagcacgagtgcctgatcggcaatacaacagtgaaggtgcacgccctggatggcaggctgggacccatgccatgtcggcctaaggagatcgtgtcctctgctggcccagtgagaaagacctcttgtaccttcaattatacaaagaccctgagaaacaagtattacgagccacgggactcttatttccagcagtatatgctgaagggggagtatcagtattggtttgacctggat

SEQ ID NO: 92 - DNA, 1053 bp, synthetic construct
  source 1..1053: mol_type = other DNA
attctgagggggcaggtcgtccagggcattatttggctgctgctggtcacaggggcacaggggagactgagttgtaaagaggattatcgctatgctatctccagcaccaacgagatcggccctctgggcgccgagggcctgaccacaacatggaaggagtacaatcacggcctgcaactggacgatggcaccgtgagggctatctgtaccgctggctcctttaaggtaatcgctctgaacgtggtgtctcgggcctatctggcttccctgcataagagagccctgccaacatctgtgaccttcgagctgctgttcgatggcacctcccctgccatcgaggagatgggcgacgattttggcttcggcctgtgccccttcgacaccacaccagtggtgaagggcaagtataacaccacactgctgaatggctccgctttctacctggtgtgccctatcggctggacaggcgtgatcgagtgtacagccgtgtctccaaccacactgagaaccgaggtggtgaaaaccttcaagagggagaagccatttccccatagggtgggctgcgtgaccacagtggtggagaaggaggatctgttctactgcaagtggggcggcaattggacctgcgtgaagggcaatcctgtgacctacatgggcggccaggtgaagcagtgtcggtggtgcggcttcgactttaaggagcctgatggcctgccacactaccccatcggcaagtgcatcctggccaatgagacaggctatagagtggtggattccacagactgcaatagagacggcgtggtaatctctaccgagggcgagcacgagtgcctgatcggcaatacaacagtgaaggtgcacgccctggatggcaggctgggacccatgccatgtcggcctaaggagatcgtgtcctctgctggcccagtgagaaagacctcttgtaccttcaattatacaaagaccctgagaaacaagtattacgagccacgggactcttatttccagcagtatatgctgaagggggagtatcagtattggtttgacctggat

SEQ ID NO: 93 - DNA, 1053 bp, synthetic construct
  source 1..1053: mol_type = other DNA
attctgagggggcaggtcgtccagggcattatttggctgctgctggtcacaggggcacaggggagactgagttgtaaagaggattatcgctatgctatctccagcaccaacgagatcggccctctgggcgccgagggcctgaccacaacatggaaggagtacaatcacggcctgcaactggacgatggcaccgtgagggctatctgtaccgctggctcctttaaggtaatcgctctgaacgtggtgtctcggtcctatctggcttccctgcataagagagccctgccaacatctgtgaccttcgagctgctgttcgatggcacctcccctgccatcgaggagatgggcgacgattttggcttcggcctgtgccccttcgacaccacaccagtggtgaagggcaagtataacaccacactgctgaatggctccgctttctacctggtgtgccctatcggctggacaggcgtgatcgagtgtacagccgtgtctccaaccacactgagaaccgaggtggtgaaaaccttcaagagggagaagccatttccccatagggtgggctgcgtgaccacagtggtggagaaggaggatctgttctactgcaagtggggcggcaattggacctgcgtgaagggcaatcctgtgacctacatgggcggccaggtgaagcagtgtcggtggtgcggcttcgactttaaggagcctgatggcctgccacactaccccatcggcaagtgcatcctggccaatgagacaggctatagagtggtggattccacagactgcaatagagacggcgtggtaatctctaccgagggcgagcacgagtgcctgatcggcaatacaacagtgaaggtgcacgccctggatggcaggctgggacccatgccatgtcggcctaaggagatcgtgtcctctgctggcccagtgagaaagacctcttgtaccttcaattatacaaagaccctgagaaacaagtattacgagccacgggactcttatttccagcagtatatgctgaagggggagtatcagtattggtttgacctggat

SEQ ID NO: 94 - DNA, 1053 bp, synthetic construct
  source 1..1053: mol_type = other DNA
attctgagggggcaggtcgtccagggcattatttggctgctgctggtcacaggggcacaggggagactgagttgtaaagaggattatcgctatgctatctccagcaccaacgagatcggccctctgggcgccgagggcctgaccacaacatggaaggagtacaatcacggcctgcaactggacgatggcaccgtgagggctatctgtaccgctggctcctttaaggtaatcgctctgaacgtggtgtctcgggagtatctggcttccctgcataagagagccctgccaacatctgtgaccttcgagctgctgttcgatggcacctcccctgccatcgaggagatgggcgacgattttggcttcggcctgtgccccttcgacaccacaccagtggtgaagggcaagtataacaccacactgctgaatggctccgctttctacctggtgtgccctatcggctggacaggcgtgatcgagtgtacagccgtgtctccaaccacactgagaaccgaggtggtgaaaaccttcaagagggagaagccatttccccatagggtgggctgcgtgaccacagtggtggagaaggaggatctgttctactgcaagtggggcggcaattggacctgcgtgaagggcaatcctgtgacctacatgggcggccaggtgaagcagtgtcggtggtgcggcttcgactttaaggagcctgatggcctgccacactaccccatcggcaagtgcatcctggccaatgagacaggctatagagtggtggattccacagactgcaatagagacggcgtggtaatctctaccgagggcgagcacgagtgcctgatcggcaatacaacagtgaaggtgcacgccctggatggcaggctgggacccatgccatgtcggcctaaggagatcgtgtcctctgctggcccagtgagaaagacctcttgtaccttcaattatacaaagaccctgagaaacaagtattacgagccacgggactcttatttccagcagtatatgctgaagggggagtatcagtattggtttgacctggat

SEQ ID NO: 95 - DNA, 1053 bp, synthetic construct
  source 1..1053: mol_type = other DNA
attctgagggggcaggtcgtccagggcattatttggctgctgctggtcacaggggcacaggggagactgagttgtaaagaggattatcgctatgctatctccagcaccaacgagatcggccctctgggcgccgagggcctgaccacaacatggaaggagtacaatcacggcctgcaactggacgatggcaccgtgagggctatctgtaccgctggctcctttaaggtaatcgctctgaacgtggtgtctcggaggtatctggcttccctgcataagagagccctgccaacatctgtgaccttcgagctgctgttcgatggcacctcccctgccatcgaggagatgggcgacgattttggcttcggcctgtgccccttcgacaccacaccagtggtgaagggcaagtataacaccacactgctgaatggctccgctttctacctggtgtgccctatcggctggacaggcgtgatcgagtgtacagccgtgtctccaaccacactgagaaccgaggtggtgaaaaccttcaagagggagaagccatttccccatagggtgggctgcgtgaccacagtggtggagaaggaggatctgttctactgcaagtggggcggcaattggacctgcgtgaagggcaatcctgtgacctacatgggcggccaggtgaagcagtgtcggtggtgcggcttcgactttaaggagcctgatggcctgccacactaccccatcggcaagtgcatcctggccaatgagacaggctatagagtggtggattccacagactgcaatagagacggcgtggtaatctctaccgagggcgagcacgagtgcctgatcggcaatacaacagtgaaggtgcacgccctggatggcaggctgggacccatgccatgtcggcctaaggagatcgtgtcctctgctggcccagtgagaaagacctcttgtaccttcaattatacaaagaccctgagaaacaagtattacgagccacgggactcttatttccagcagtatatgctgaagggggagtatcagtattggtttgacctggat

SEQ ID NO: 96 - protein, 562 aa, synthetic construct
RLSCKEDYRYAISKTNEIGPLGPTGLTTRWHAYIKGLHITDSSVDLTCVDGSFLVYKRCVKKQRYLATVHERALSTSVEFTLVAEPQDLEEVQMGDDFEFGLCPCDSTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLDGGSTKTKPPCPICPACESPGPSVFIFPPKPKDTLMISRTPQVTCVVVDVSQENPEVQFSWYVDGVEVHTAQTRPKEEQFNSTYRVVSVLPIQHQDWLNGKEFKCKVNNKDLPAPITRIISKAKGQTREPQVYTLPPHAEELSRSKVSITCLVIGFYPPDIDVEWQRNGQPEPEGNYRTTPPQQDVDGTYFLYSKFSVDKASWQGGGIFQCAVMHEALHNHYTQKSISKTPGK

SEQ ID NO: 97 - protein, 562 aa, synthetic construct
RLSCKEDYRYAISETNEIGPLGPTGLTTRWHAYIKGLHITDSSVDLTCVDGSFLVYKRCVKKQKYLATVHERALSTSVEFTLVAEPQDLEEVQMGDDFEFGLCPCDSTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLDGGSTKTKPPCPICPACESPGPSVFIFPPKPKDTLMISRTPQVTCVVVDVSQENPEVQFSWYVDGVEVHTAQTRPKEEQFNSTYRVVSVLPIQHQDWLNGKEFKCKVNNKDLPAPITRIISKAKGQTREPQVYTLPPHAEELSRSKVSITCLVIGFYPPDIDVEWQRNGQPEPEGNYRTTPPQQDVDGTYFLYSKFSVDKASWQGGGIFQCAVMHEALHNHYTQKSISKTPGK

SEQ ID NO: 98 - protein, 562 aa, synthetic construct
RLSCKEDYRYAISETNEIGPLGPRGLTTRWHAYIKGLHITDSSVDLTCVDGSFLVYKRCVKKQRYLATVHERALSTSVEFTLVAEPQDLEEVQMGDDFEFGLCPCDSTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLDGGSTKTKPPCPICPACESPGPSVFIFPPKPKDTLMISRTPQVTCVVVDVSQENPEVQFSWYVDGVEVHTAQTRPKEEQFNSTYRVVSVLPIQHQDWLNGKEFKCKVNNKDLPAPITRIISKAKGQTREPQVYTLPPHAEELSRSKVSITCLVIGFYPPDIDVEWQRNGQPEPEGNYRTTPPQQDVDGTYFLYSKFSVDKASWQGGGIFQCAVMHEALHNHYTQKSISKTPGK

SEQ ID NO: 99 - protein, 562 aa, synthetic construct
RLSCKEDYRYAISETNEIGPLGPTDLTTRWHAYIKGLHITDSSVDLTCVDGSFLVYKRCVKKQRYLATVHERALSTSVEFTLVAEPQDLEEVQMGDDFEFGLCPCDSTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLDGGSTKTKPPCPICPACESPGPSVFIFPPKPKDTLMISRTPQVTCVVVDVSQENPEVQFSWYVDGVEVHTAQTRPKEEQFNSTYRVVSVLPIQHQDWLNGKEFKCKVNNKDLPAPITRIISKAKGQTREPQVYTLPPHAEELSRSKVSITCLVIGFYPPDIDVEWQRNGQPEPEGNYRTTPPQQDVDGTYFLYSKFSVDKASWQGGGIFQCAVMHEALHNHYTQKSISKTPGK

SEQ ID NO: 100 - protein, 562 aa, synthetic construct
RLSCKEDYRYAISETNEIGPLGPTNLTTRWHAYIKGLHITDSSVDLTCVDGSFLVYKRCVKKQRYLATVHERALSTSVEFTLVAEPQDLEEVQMGDDFEFGLCPCDSTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLDGGSTKTKPPCPICPACESPGPSVFIFPPKPKDTLMISRTPQVTCVVVDVSQENPEVQFSWYVDGVEVHTAQTRPKEEQFNSTYRVVSVLPIQHQDWLNGKEFKCKVNNKDLPAPITRIISKAKGQTREPQVYTLPPHAEELSRSKVSITCLVIGFYPPDIDVEWQRNGQPEPEGNYRTTPPQQDVDGTYFLYSKFSVDKASWQGGGIFQCAVMHEALHNHYTQKSISKTPGK

SEQ ID NO: 101 - protein, 562 aa, synthetic construct
RLSCKEDYRYAISETNEIGPLGPTRLTTRWHAYIKGLHITDSSVDLTCVDGSFLVYKRCVKKQRYLATVHERALSTSVEFTLVAEPQDLEEVQMGDDFEFGLCPCDSTPVVKGKYNTTLLNGSAFYLVCPIGWTGVIECTAVSPTTLRTEVVKTFKREKPFPHRVGCVTTVVEKEDLFYCKWGGNWTCVKGNPVTYMGGQVKQCRWCGFDFKEPDGLPHYPIGKCILANETGYRVVDSTDCNRDGVVISTEGEHECLIGNTTVKVHALDGRLGPMPCRPKEIVSSAGPVRKTSCTFNYTKTLRNKYYEPRDSYFQQYMLKGEYQYWFDLDGGSTKTKPPCPICPACESPGPSVFIFPPKPKDTLMISRTPQVTCVVVDVSQENPEVQFSWYVDGVEVHTAQTRPKEEQFNSTYRVVSVLPIQHQDWLNGKEFKCKVNNKDLPAPITRIISKAKGQTREPQVYTLPPHAEELSRSKVSITCLVIGFYPPDIDVEWQRNGQPEPEGNYRTTPPQQDVDGTYFLYSKFSVDKASWQGGGIFQCAVMHEALHNHYTQKSISKTPGK

SEQ ID NO: 102 - DNA, 1752 bp, synthetic construct
  source 1..1752: mol_type = other DNA
atgattctgagggggcaggtcgtccagggcattatttggctgctgctggtcacaggggcacaggggagactgagttgtaaagaggattatcgctatgctatctccaagaccaacgagatcggccctctgggccccaccggcctgaccacaaggtggcacgcctacatcaagggcctgcacatcaccgattcctccgtggacctgacctgtgtggacggctcctttctggtatacaagaggtgcgtgaagaagcagaggtatctggctaccgtgcatgagagagccctgtccacatctgtggagttcaccctggtggccgagccccaggacctggaggaggtgcagatgggcgacgattttgagttcggcctgtgcccctgcgactccacaccagtggtgaagggcaagtataacaccacactgctgaatggctccgctttctacctggtgtgccctatcggctggacaggcgtgatcgagtgtacagccgtgtctccaaccacactgagaaccgaggtggtgaaaaccttcaagagggagaagccatttccccatagggtgggctgcgtgaccacagtggtggagaaggaggatctgttctactgcaagtggggcggcaattggacctgcgtgaagggcaatcctgtgacctacatgggcggccaggtgaagcagtgtcggtggtgcggcttcgactttaaggagcctgatggcctgccacactaccccatcggcaagtgcatcctggccaatgagacaggctatagagtggtggattccacagactgcaatagagacggcgtggtaatctctaccgagggcgagcacgagtgcctgatcggcaatacaacagtgaaggtgcacgccctggatggcaggctgggacccatgccatgtcggcctaaggagatcgtgtcctctgctggcccagtgagaaagacctcttgtaccttcaattatacaaagaccctgagaaacaagtattacgagccacgggactcttatttccagcagtatatgctgaagggggagtatcagtattggtttgacctggatggcggctcaaccaaaactaaaccaccctgccctatctgccctgcctgcgagtctcccggaccttcagtcttcattttcccccctaagcccaaggacaccctgatgatcagcagaacccctcaggtgacatgcgtggtggtggacgtgtcccaggagaaccctgaggtgcagttcagctggtacgtggatggcgtggaggtgcacaccgcccagacaagaccaaaggaggagcagtttaacagcacatatcgcgtggtgtctgtgctgcccatccagcatcaggactggctgaatggcaaggagttcaagtgcaaggtgaacaataaggatctgccagcccccatcaccaggatcatctctaaggctaagggccagacccgggagccacaggtgtacacactgccaccacacgctgaggagctgtcccgcagcaaggtgtccatcacatgtctggtcatcggcttttaccctccagacatcgatgtggagtggcagaggaacggccagcctgagccagagggcaattatcggaccacaccccctcagcaggacgtggatggcacctacttcctgtattctaagttttccgtggataaggcctcttggcagggcggcggcatcttccagtgtgctgtgatgcacgaagcactgcacaaccattacactcagaaaagtatctctaagaccccaggaaaa

SEQ ID NO: 103 - DNA, 1752 bp, synthetic construct
  source 1..1752: mol_type = other DNA
atgattctgagggggcaggtcgtccagggcattatttggctgctgctggtcacaggggcacaggggagactgagttgtaaagaggattatcgctatgctatctccgagaccaacgagatcggccctctgggccccaccggcctgaccacaaggtggcacgcctacatcaagggcctgcacatcaccgattcctccgtggacctgacctgtgtggacggctcctttctggtatacaagaggtgcgtgaagaagcagaagtatctggctaccgtgcatgagagagccctgtccacatctgtggagttcaccctggtggccgagccccaggacctggaggaggtgcagatgggcgacgattttgagttcggcctgtgcccctgcgactccacaccagtggtgaagggcaagtataacaccacactgctgaatggctccgctttctacctggtgtgccctatcggctggacaggcgtgatcgagtgtacagccgtgtctccaaccacactgagaaccgaggtggtgaaaaccttcaagagggagaagccatttccccatagggtgggctgcgtgaccacagtggtggagaaggaggatctgttctactgcaagtggggcggcaattggacctgcgtgaagggcaatcctgtgacctacatgggcggccaggtgaagcagtgtcggtggtgcggcttcgactttaaggagcctgatggcctgccacactaccccatcggcaagtgcatcctggccaatgagacaggctatagagtggtggattccacagactgcaatagagacggcgtggtaatctctaccgagggcgagcacgagtgcctgatcggcaatacaacagtgaaggtgcacgccctggatggcaggctgggacccatgccatgtcggcctaaggagatcgtgtcctctgctggcccagtgagaaagacctcttgtaccttcaattatacaaagaccctgagaaacaagtattacgagccacgggactcttatttccagcagtatatgctgaagggggagtatcagtattggtttgacctggatggcggctcaaccaaaactaaaccaccctgccctatctgccctgcctgcgagtctcccggaccttcagtcttcattttcccccctaagcccaaggacaccctgatgatcagcagaacccctcaggtgacatgcgtggtggtggacgtgtcccaggagaaccctgaggtgcagttcagctggtacgtggatggcgtggaggtgcacaccgcccagacaagaccaaaggaggagcagtttaacagcacatatcgcgtggtgtctgtgctgcccatccagcatcaggactggctgaatggcaaggagttcaagtgcaaggtgaacaataaggatctgccagcccccatcaccaggatcatctctaaggctaagggccagacccgggagccacaggtgtacacactgccaccacacgctgaggagctgtcccgcagcaaggtgtccatcacatgtctggtcatcggcttttaccctccagacatcgatgtggagtggcagaggaacggccagcctgagccagagggcaattatcggaccacaccccctcagcaggacgtggatggcacctacttcctgtattctaagttttccgtggataaggcctcttggcagggcggcggcatcttccagtgtgctgtgatgcacgaagcactgcacaaccattacactcagaaaagtatctctaagaccccaggaaaa

SEQ ID NO: 104 - DNA, 1752 bp, synthetic construct
  source 1..1752: mol_type = other DNA
atgattctgagggggcaggtcgtccagggcattatttggctgctgctggtcacaggggcacaggggagactgagttgtaaagaggattatcgctatgctatctccgagaccaacgagatcggccctctgggccccaggggcctgaccacaaggtggcacgcctacatcaagggcctgcacatcaccgattcctccgtggacctgacctgtgtggacggctcctttctggtatacaagaggtgcgtgaagaagcagaggtatctggctaccgtgcatgagagagccctgtccacatctgtggagttcaccctggtggccgagccccaggacctggaggaggtgcagatgggcgacgattttgagttcggcctgtgcccctgcgactccacaccagtggtgaagggcaagtataacaccacactgctgaatggctccgctttctacctggtgtgccctatcggctggacaggcgtgatcgagtgtacagccgtgtctccaaccacactgagaaccgaggtggtgaaaaccttcaagagggagaagccatttccccatagggtgggctgcgtgaccacagtggtggagaaggaggatctgttctactgcaagtggggcggcaattggacctgcgtgaagggcaatcctgtgacctacatgggcggccaggtgaagcagtgtcggtggtgcggcttcgactttaaggagcctgatggcctgccacactaccccatcggcaagtgcatcctggccaatgagacaggctatagagtggtggattccacagactgcaatagagacggcgtggtaatctctaccgagggcgagcacgagtgcctgatcggcaatacaacagtgaaggtgcacgccctggatggcaggctgggacccatgccatgtcggcctaaggagatcgtgtcctctgctggcccagtgagaaagacctcttgtaccttcaattatacaaagaccctgagaaacaagtattacgagccacgggactcttatttccagcagtatatgctgaagggggagtatcagtattggtttgacctggatggcggctcaaccaaaactaaaccaccctgccctatctgccctgcctgcgagtctcccggaccttcagtcttcattttcccccctaagcccaaggacaccctgatgatcagcagaacccctcaggtgacatgcgtggtggtggacgtgtcccaggagaaccctgaggtgcagttcagctggtacgtggatggcgtggaggtgcacaccgcccagacaagaccaaaggaggagcagtttaacagcacatatcgcgtggtgtctgtgctgcccatccagcatcaggactggctgaatggcaaggagttcaagtgcaaggtgaacaataaggatctgccagcccccatcaccaggatcatctctaaggctaagggccagacccgggagccacaggtgtacacactgccaccacacgctgaggagctgtcccgcagcaaggtgtccatcacatgtctggtcatcggcttttaccctccagacatcgatgtggagtggcagaggaacggccagcctgagccagagggcaattatcggaccacaccccctcagcaggacgtggatggcacctacttcctgtattctaagttttccgtggataaggcctcttggcagggcggcggcatcttccagtgtgctgtgatgcacgaagcactgcacaaccattacactcagaaaagtatctctaagaccccaggaaaa

SEQ ID NO: 105 - DNA, 1752 bp, synthetic construct
  source 1..1752: mol_type = other DNA
atgattctgagggggcaggtcgtccagggcattatttggctgctgctggtcacaggggcacaggggagactgagttgtaaagaggattatcgctatgctatctccgagaccaacgagatcggccctctgggccccaccgacctgaccacaaggtggcacgcctacatcaagggcctgcacatcaccgattcctccgtggacctgacctgtgtggacggctcctttctggtatacaagaggtgcgtgaagaagcagaggtatctggctaccgtgcatgagagagccctgtccacatctgtggagttcaccctggtggccgagccccaggacctggaggaggtgcagatgggcgacgattttgagttcggcctgtgcccctgcgactccacaccagtggtgaagggcaagtataacaccacactgctgaatggctccgctttctacctggtgtgccctatcggctggacaggcgtgatcgagtgtacagccgtgtctccaaccacactgagaaccgaggtggtgaaaaccttcaagagggagaagccatttccccatagggtgggctgcgtgaccacagtggtggagaaggaggatctgttctactgcaagtggggcggcaattggacctgcgtgaagggcaatcctgtgacctacatgggcggccaggtgaagcagtgtcggtggtgcggcttcgactttaaggagcctgatggcctgccacactaccccatcggcaagtgcatcctggccaatgagacaggctatagagtggtggattccacagactgcaatagagacggcgtggtaatctctaccgagggcgagcacgagtgcctgatcggcaatacaacagtgaaggtgcacgccctggatggcaggctgggacccatgccatgtcggcctaaggagatcgtgtcctctgctggcccagtgagaaagacctcttgtaccttcaattatacaaagaccctgagaaacaagtattacgagccacgggactcttatttccagcagtatatgctgaagggggagtatcagtattggtttgacctggatggcggctcaaccaaaactaaaccaccctgccctatctgccctgcctgcgagtctcccggaccttcagtcttcattttcccccctaagcccaaggacaccctgatgatcagcagaacccctcaggtgacatgcgtggtggtggacgtgtcccaggagaaccctgaggtgcagttcagctggtacgtggatggcgtggaggtgcacaccgcccagacaagaccaaaggaggagcagtttaacagcacatatcgcgtggtgtctgtgctgcccatccagcatcaggactggctgaatggcaaggagttcaagtgcaaggtgaacaataaggatctgccagcccccatcaccaggatcatctctaaggctaagggccagacccgggagccacaggtgtacacactgccaccacacgctgaggagctgtcccgcagcaaggtgtccatcacatgtctggtcatcggcttttaccctccagacatcgatgtggagtggcagaggaacggccagcctgagccagagggcaattatcggaccacaccccctcagcaggacgtggatggcacctacttcctgtattctaagttttccgtggataaggcctcttggcagggcggcggcatcttccagtgtgctgtgatgcacgaagcactgcacaaccattacactcagaaaagtatctctaagaccccaggaaaa

SEQ ID NO: 106 - DNA, 1752 bp, synthetic construct
  source 1..1752: mol_type = other DNA
atgattctgagggggcaggtcgtccagggcattatttggctgctgctggtcacaggggcacaggggagactgagttgtaaagaggattatcgctatgctatctccgagaccaacgagatcggccctctgggccccaccaacctgaccacaaggtggcacgcctacatcaagggcctgcacatcaccgattcctccgtggacctgacctgtgtggacggctcctttctggtatacaagaggtgcgtgaagaagcagaggtatctggctaccgtgcatgagagagccctgtccacatctgtggagttcaccctggtggccgagccccaggacctggaggaggtgcagatgggcgacgattttgagttcggcctgtgcccctgcgactccacaccagtggtgaagggcaagtataacaccacactgctgaatggctccgctttctacctggtgtgccctatcggctggacaggcgtgatcgagtgtacagccgtgtctccaaccacactgagaaccgaggtggtgaaaaccttcaagagggagaagccatttccccatagggtgggctgcgtgaccacagtggtggagaaggaggatctgttctactgcaagtggggcggcaattggacctgcgtgaagggcaatcctgtgacctacatgggcggccaggtgaagcagtgtcggtggtgcggcttcgactttaaggagcctgatggcctgccacactaccccatcggcaagtgcatcctggccaatgagacaggctatagagtggtggattccacagactgcaatagagacggcgtggtaatctctaccgagggcgagcacgagtgcctgatcggcaatacaacagtgaaggtgcacgccctggatggcaggctgggacccatgccatgtcggcctaaggagatcgtgtcctctgctggcccagtgagaaagacctcttgtaccttcaattatacaaagaccctgagaaacaagtattacgagccacgggactcttatttccagcagtatatgctgaagggggagtatcagtattggtttgacctggatggcggctcaaccaaaactaaaccaccctgccctatctgccctgcctgcgagtctcccggaccttcagtcttcattttcccccctaagcccaaggacaccctgatgatcagcagaacccctcaggtgacatgcgtggtggtggacgtgtcccaggagaaccctgaggtgcagttcagctggtacgtggatggcgtggaggtgcacaccgcccagacaagaccaaaggaggagcagtttaacagcacatatcgcgtggtgtctgtgctgcccatccagcatcaggactggctgaatggcaaggagttcaagtgcaaggtgaacaataaggatctgccagcccccatcaccaggatcatctctaaggctaagggccagacccgggagccacaggtgtacacactgccaccacacgctgaggagctgtcccgcagcaaggtgtccatcacatgtctggtcatcggcttttaccctccagacatcgatgtggagtggcagaggaacggccagcctgagccagagggcaattatcggaccacaccccctcagcaggacgtggatggcacctacttcctgtattctaagttttccgtggataaggcctcttggcagggcggcggcatcttccagtgtgctgtgatgcacgaagcactgcacaaccattacactcagaaaagtatctctaagaccccaggaaaa

SEQ ID NO: 107 - DNA, 1752 bp, synthetic construct
  source 1..1752: mol_type = other DNA
atgattctgagggggcaggtcgtccagggcattatttggctgctgctggtcacaggggcacaggggagactgagttgtaaagaggattatcgctatgctatctccgagaccaacgagatcggccctctgggccccaccaggctgaccacaaggtggcacgcctacatcaagggcctgcacatcaccgattcctccgtggacctgacctgtgtggacggctcctttctggtatacaagaggtgcgtgaagaagcagaggtatctggctaccgtgcatgagagagccctgtccacatctgtggagttcaccctggtggccgagccccaggacctggaggaggtgcagatgggcgacgattttgagttcggcctgtgcccctgcgactccacaccagtggtgaagggcaagtataacaccacactgctgaatggctccgctttctacctggtgtgccctatcggctggacaggcgtgatcgagtgtacagccgtgtctccaaccacactgagaaccgaggtggtgaaaaccttcaagagggagaagccatttccccatagggtgggctgcgtgaccacagtggtggagaaggaggatctgttctactgcaagtggggcggcaattggacctgcgtgaagggcaatcctgtgacctacatgggcggccaggtgaagcagtgtcggtggtgcggcttcgactttaaggagcctgatggcctgccacactaccccatcggcaagtgcatcctggccaatgagacaggctatagagtggtggattccacagactgcaatagagacggcgtggtaatctctaccgagggcgagcacgagtgcctgatcggcaatacaacagtgaaggtgcacgccctggatggcaggctgggacccatgccatgtcggcctaaggagatcgtgtcctctgctggcccagtgagaaagacctcttgtaccttcaattatacaaagaccctgagaaacaagtattacgagccacgggactcttatttccagcagtatatgctgaagggggagtatcagtattggtttgacctggatggcggctcaaccaaaactaaaccaccctgccctatctgccctgcctgcgagtctcccggaccttcagtcttcattttcccccctaagcccaaggacaccctgatgatcagcagaacccctcaggtgacatgcgtggtggtggacgtgtcccaggagaaccctgaggtgcagttcagctggtacgtggatggcgtggaggtgcacaccgcccagacaagaccaaaggaggagcagtttaacagcacatatcgcgtggtgtctgtgctgcccatccagcatcaggactggctgaatggcaaggagttcaagtgcaaggtgaacaataaggatctgccagcccccatcaccaggatcatctctaaggctaagggccagacccgggagccacaggtgtacacactgccaccacacgctgaggagctgtcccgcagcaaggtgtccatcacatgtctggtcatcggcttttaccctccagacatcgatgtggagtggcagaggaacggccagcctgagccagagggcaattatcggaccacaccccctcagcaggacgtggatggcacctacttcctgtattctaagttttccgtggataaggcctcttggcagggcggcggcatcttccagtgtgctgtgatgcacgaagcactgcacaaccattacactcagaaaagtatctctaagaccccaggaaaa

(19) United States
(12) Patent Application Publication
(10) Pub. No.: US 2025/0230194 A1
(43) Pub. Date: Jul. 17, 2025

(54) RECOMBINANT CLASSICAL SWINE FEVER VIRUS E2 PROTEIN WITH B/C DOMAIN SWAPPING

(71) Applicant: BOEHRINGER INGELHEIM VETMEDICA (CHINA) CO., LTD., Taizhou, Jiangsu (CN)

(72) Inventors: Ning CHEN, Shanghai (CN); Huanhuan LIU, Shanghai (CN); Jiaying WANG, Shanghai (CN); Chao TONG, Shanghai (CN)

(21) Appl. No.: 18/702,769

(22) PCT Filed: Oct. 18, 2022
(86) PCT No.: PCT/CN2022/125849

§ 371 Date: Apr. 18, 2024

(30) Foreign Application Priority Data
Oct. 19, 2021 (WO) PCT/CN2021/124797

(51) Int. Cl. C07K 14/005; A61K 39/00; A61K 39/12; G01N 33/569

(52) U.S. Cl. CPC C07K 14/005; A61K 39/12; G01N 33/56983; A61K 2039/552; C12N 2770/24322; C12N 2770/24334; C12N 2770/24351; G01N 2333/183

(57) ABSTRACT

The present invention relates the field of animal health. Particularly, the present invention relates to a recombinant classical swine fever virus E2 protein in which a fragment comprising at least one of the amino acids defining the 6B8 epitope of the CSFV E2 protein is replaced by a corresponding fragment of the E2 protein from a pestivirus other than CSFV. Further, the present invention provides an immunogenic composition comprising the recombinant E2 protein of the present invention and the use of the immunogenic composition for preventing and/or treating diseases associated with CSFV in an animal. Moreover, the present invention provides a method and a kit for differentiating animals infected with CSFV from animals vaccinated with the immunogenic composition of the present invention.

### TECHNICAL FIELD

The present invention relates to the field of animal health. Particularly, the present invention relates to a recombinant classical swine fever virus (CSFV) E2 protein in which a fragment comprising at least one of the amino acids defining the 6B8 epitope of the CSFV E2 protein is replaced by a corresponding fragment of the E2 protein from a pestivirus other than CSFV. Further, the present invention provides an immunogenic composition comprising the recombinant E2 protein of the present invention and the use of the immunogenic composition for preventing and/or treating diseases associated with CSFV in an animal. Further, the present invention provides a recombinant CSFV, preferably in attenuated form, that comprises the recombinant E2 protein of the present invention. Moreover, the present invention provides a method and a kit for differentiating animals infected with CSFV from animals vaccinated with the immunogenic composition of the present invention.

### TECHNICAL BACKGROUND

Classical swine fever (CSF) is a highly contagious disease of pigs and wild boars that causes significant economic losses. The causative agent of the disease is classical swine fever virus (CSFV). In China, a combination of prophylactic vaccination and stamping out strategy is implemented to control CSF outbreaks. However, sporadic CSF outbreaks and persistent infection are still reported in most parts of China.

There is still a need in the art for a new CSFV vaccine that is safe, effective and animals vaccinated by which can be differentiated from those infected by wild type field strains.

### BRIEF DESCRIPTION OF THE INVENTION

In one aspect, the present invention provides a recombinant CSFV (classical swine fever virus) E2 protein in which a fragment comprising at least one of the amino acids defining the 6B8 epitope of the CSFV E2 protein is replaced by a corresponding fragment of the E2 protein from a pestivirus other than CSFV.

In one aspect, the present invention provides a recombinant CSFV E2 protein, wherein the recombinant CSFV E2 protein comprises an amino acid sequence of the E2 protein of CSFV field strain QZ07 with a fragment comprising at least one of the amino acids defining the 6B8 epitope being replaced by a corresponding fragment of the E2 protein of BVDV3 Hobi-like Th/04 KhonKaen strain.

In one aspect, the present invention provides a recombinant CSFV E2 protein, wherein the recombinant CSFV E2 protein comprises an amino acid sequence of the E2 protein of CSFV field strain QZ07 with the sequence of amino acid position 11 to amino acid position 109 of the E2 protein of CSFV field strain QZ07 being replaced by a corresponding fragment of the E2 protein of BVDV3 Hobi-like Th/04 KhonKaen strain, and the numbering of the amino acid position is determined based on the position numbers of the amino acid sequence of the E2 protein of CSFV field strain QZ07 shown by SEQ ID NO: 10.

In one aspect, the recombinant CSFV E2 protein wherein the sequence of amino acid position 11 to amino acid position 109 of the E2 protein of CSFV field strain QZ07 being replaced by a corresponding fragment of the E2 protein of BVDV3 Hobi-like Th/04 KhonKaen strain, is further linked to an immunoglobulin Fc fragment.

In one aspect, the recombinant CSFV E2 protein wherein the sequence of amino acid position 11 to amino acid position 109 of the E2 protein of CSFV field strain QZ07 being replaced by a corresponding fragment of the E2 protein of BVDV3 Hobi-like Th/04 KhonKaen strain, including the recombinant CSFV E2 protein linked to an immunoglobulin Fc fragment, comprises further mutations at amino acid positions 10, 14, 22, 24, 25, 24/25, 41 and/or 64 of the CSFV E2 protein as further defined in the specification. In one aspect, the present invention provides a recombinant nucleic acid coding for the recombinant CSFV E2 protein of the present invention.

In one aspect, the present invention provides a vector comprising the nucleic acid of the present invention.

In one aspect, the present invention provides a host cell comprising the nucleic acid of the present invention or the vector of the present invention.

In one aspect, the present invention provides a method for producing the recombinant CSFV E2 protein of the present invention, comprising (i) culturing the host cell of the present invention under conditions suitable for the expression of the CSFV E2 protein, and (ii) isolating and optionally purifying the CSFV E2 protein.

In one aspect, the present invention provides a recombinant CSFV (classical swine fever virus) comprising the recombinant CSFV E2 protein of the present invention.

In one aspect, the present invention provides an immunogenic composition comprising the recombinant CSFV E2 protein of the present invention, the recombinant nucleic acid of the present invention, the vector of the present invention, and/or the recombinant CSFV of the present invention.

In one aspect, the present invention provides a method of preventing and/or treating diseases associated with CSFV in an animal, the method comprising the step of administering the immunogenic composition of the present invention to an animal in need thereof.

In one aspect, the present invention provides a method of differentiating animals infected with CSFV from animals vaccinated with the immunogenic composition of the present invention, comprising a) obtaining a sample, and b) testing said sample in an immuno test.

In one aspect, the present invention provides a kit for differentiating animals infected with CSFV from animals vaccinated with the immunogenic composition of the present invention, which comprises an antibody specifically recognizing the 6B8 epitope of the CSFV E2 protein or an antigen-binding fragment thereof.

In one aspect, the present invention provides a method for producing the recombinant CSFV E2 protein as described herein in CHO cells, said method comprises a) adapting CHO cells to high-density suspension culture in a medium; b) transfecting the adapted CHO cells from step a) with a mammalian expression vector comprising the nucleic acid molecule encoding the recombinant CSFV E2 protein of the present invention, c) culturing the transfected CHO cells from step b) under conditions suitable for the expression of the recombinant CSFV E2 protein; and d) harvesting and optionally purifying the recombinant CSFV E2 protein.

In one aspect, the present invention provides a method for producing the recombinant CSFV E2 protein as described herein in CHO cells, said method comprises a) growing CHO cells in a medium; b) transfecting the CHO cells from step a) with a mammalian expression vector comprising the nucleic acid molecule encoding the recombinant CSFV E2 protein of the present invention, c) culturing the transfected CHO cells from step b) under conditions suitable for the expression of the recombinant

CSFV E2 protein; and d) harvesting and optionally purifying the recombinant CSFV E2 protein.

### BRIEF DESCRIPTION OF THE DRAWINGS

FIG. 1: Phylogenetic analysis and classification of pestiviruses (modified from Peterhans, et al., Vet. Res. Vol. 41(6).).

FIG. 2: Structures of the recombinant CSFV E2 proteins.

FIG. 3: Western blot results of QZ07E2-Reindeer V60 11-110 Chimeric FC, QZ07E2-Reindeer V60 11-56 KARD Chimeric FC, QZ07E2-PG2 11-56 Chimeric FC, QZ07E2-TSV9552 11-38 KARD Chimeric FC, QZ07E2-TSV9552 11-109 Chimeric FC, QZ07E2-Hobi-like 11-109 Chimeric FC, QZ07E2-Hobi-like 11-56 Chimeric FC and QZ07E2-Reindeer V60 11-80 Chimeric FC.

FIG. 4: Identification of additional critical residues for 6B8 binding in E2. IFA detection of mutation forms of E2 protein in CHO cell expression system shows that amino acid residues at position 10, 41, or 64 are critical for mAb 6B8 binding.

FIG. 5: Western blot detection of mutation forms of E2 protein in CHO cell expression system.

FIG. 6: Identification of additional amino acids at position 22 for inhibiting 6B8 binding in E2.

FIG. 7: Identification of additional amino acids at position 24 for inhibiting 6B8 binding in E2.

FIG. 8: Identification of additional amino acids at position 25 for inhibiting 6B8 binding in E2.

FIG. 9: Identification of additional amino acids at position 64 for inhibiting 6B8 binding in E2.

FIG. 10: Identification of additional amino acids at position 14 for inhibiting 6B8 binding in E2.

FIG. 11: Structures of the recombinant CSFV E2 proteins expressed from the constructs of QZ07E2-Hobi-like 11-109 Chimeric E14K FC, QZ07E2-Hobi-like 11-109 Chimeric R64K FC, QZ07E2-Hobi-like 11-109 Chimeric T24R FC, QZ07E2-Hobi-like 11-109 Chimeric G25D FC, QZ07E2-Hobi-like 11-109 Chimeric G25N FC, and QZ07E2-Hobi-like 11-109 Chimeric G25R FC.

FIG. 12: Western blot results of the supernatants prepared from the expression of the constructs of QZ07E2-Hobi-like 11-109 Chimeric E14K FC, QZ07E2-Hobi-like 11-109 Chimeric T24R FC, QZ07E2-Hobi-like 11-109 Chimeric R64K FC, QZ07E2-Hobi-like 11-109 Chimeric G25D FC, QZ07E2-Hobi-like 11-109 Chimeric G25N FC, QZ07E2-Hobi-like 11-109 Chimeric G25R FC, QZ07E2-Hobi-like 11-109 Chimeric FC, and QZ07E2-WT 330 FC.

### DETAILED DESCRIPTION

Before the aspects of the present invention are described, it must be noted that as used herein and in the appended claims, the singular forms “a”, “an”, and “the” include plural reference unless the context clearly dictates otherwise. Thus, for example, reference to “a or an epitope” includes a plurality of epitopes, reference to the “virus” is a reference to one or more viruses and equivalents thereof known to those skilled in the art, and so forth. The term “and/or” is intended to encompass any combinations of the items connected by this term, equivalent to listing all the combinations individually. For example, “A, B and/or C” encompasses “A”, “B”, “C”, “A and B”, “A and C”, “B and C”, and “A and B and C”. Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications mentioned herein are incorporated herein by reference for the purpose of describing and disclosing the virus strains, the cell lines, vectors, and methodologies as reported in the publications which might be used in connection with the invention. Nothing herein is to be construed as an admission that the invention is not entitled to antedate such disclosure by virtue of prior invention.

A core feature of a desired new vaccine in the field of animal health, in particular in the CSFV subunit vaccine, is its ability to differentiate infected animal from vaccinated animal (DIVA). The DIVA feature will be an essential improvement from the traditional CSFV E2 subunit vaccine and has important technical advantage.

The inventors have developed a strategy of introducing DIVA feature to alter one or more critical epitope in the immune dominant E2 protein surface and using ELISA to demonstrate the absence of antibody recognizing wild type epitope as an indication of vaccination (negative DIVA) (the entire contents of WO2020211802A are incorporated by reference herein). To implement this strategy, the inventors have chosen the strongly neutralizing mouse mAb 6B8. The 6B8 antibody recognizes and binds to the 6B8 epitope within the B/C domain of the E2 protein which comprises at least the amino acids at positions 24, 25, 14, 22, 10, 41 and/or 64.

In one aspect, the present invention provides a recombinant CSFV E2 protein in which a fragment comprising at least one of the amino acids defining the 6B8 epitope of the CSFV E2 protein is replaced by a corresponding fragment of the E2 protein from a pestivirus other than CSFV, which is not capable to bind the 6B8 epitope.

The term “CSFV” as used herein refers to all viruses belonging to species of classical swine fever virus (CSFV) in the genus Pestivirus within the family Flaviviridae.

The term “recombinant” refers to a protein or a nucleic acid that has been altered, rearranged, or modified by genetic engineering. However, the term does not refer to alterations in polynucleotide, amino acid sequence, nucleotide sequence that result from naturally occurring events, such as spontaneous mutations. The term “chimeric” refers to a protein or a nucleic acid of a species comprises a protein or a nucleic acid from other species. The term “chimeric” can also be considered as an example of “recombinant”.

In one aspect, the recombinant CSFV E2 protein is isolated.

A polypeptide or nucleic acid molecule is considered to be “isolated”—for example, when compared to its native biological source and/or the reaction medium or cultivation medium from which it has been obtained-when it has been separated from at least one other component with which it is usually associated in said source or medium, such as another protein/polypeptide, another nucleic acid, another biological component or macromolecule or at least one contaminant, impurity or minor component. In particular, a polypeptide or nucleic acid molecule is considered “isolated” when it has been purified at least 2-fold, in particular at least 10-fold, more in particular at least 100-fold, and up to 1000-fold or more. A polypeptide or nucleic acid molecule that is “in isolated form” is preferably essentially homogeneous, as determined using a suitable technique, such as a suitable chromatographical technique, such as polyacrylamide gel electrophoresis.

The term “CSFV E2 protein” refers to the processed E2 protein which results as final cleavage product from the polyprotein (Npro-C-Erns-E1-E2-p7-NS2-NS3-NS4A-NS4B-NS5A-NS5B) of the CSFV. A person skilled in the art would acknowledge that any E2 protein of CSFV can be used in the invention. In one aspect of the invention, the recombinant CSFV E2 protein is derived from a wildtype CSFV E2 protein having a 6B8 epitope specifically recognized by the 6B8 monoclonal antibody. For example, the CSFV E2 protein can be derived from known CSFV strains such as C-strain, QZ07, GD18 or GD191. For example, the E2 protein of the field strain QZ07 has the amino acid sequence set forth in SEQ ID NO:10, the E2 protein of the field strain GD18 has the amino acid sequence set forth in SEQ ID NO:11, the E2 protein of the field strain GD191 has the amino acid sequence set forth in SEQ ID NO:12, and the E2 protein of C-strain has the amino acid sequence set forth in SEQ ID NO:9.

As used in the present invention, the numbering of amino acid residue refers to the amino acid position in the processed CSFV E2 protein from the N-terminus, e.g., to the amino acid position as provided in SEQ ID NO: 10 unless otherwise indicated specifically.

“The 6B8 epitope of the CSFV E2 protein” herein refers to an epitope of the CSFV E2 protein specifically recognized and/or bound by the 6B8 monoclonal antibody as disclosed herein. The 6B8 epitope may be a conformational epitope. The 6B8 epitope may comprise defined amino acids at positions 24, 25, 14, 22, 10, 41 and/or 64 of the CSFV E2 protein. For example, the 6B8 epitope may at least comprise defined amino acids at positions 14, 22, and 24/25 of the CSFV E2 protein. The amino acid positions defining the 6B8 epitope of the CSFV E2 protein at least comprises position 14, position 22, position 24, positions 24/25, position 10, position 41 and/or position 64 of the E2 protein. For example, the 6B8 epitope may comprise S at position 14, G at position 22, E or G at position 24, G at position 25, Y at position 10, D at position 41, and/or R at position 64 of the E2 protein.

The term “6B8 monoclonal antibody” or “mAb 6B8” refers to the 6B8 monoclonal antibody or an antigen-binding fragment thereof, wherein the 6B8 monoclonal antibody specifically recognizes the 6B8 epitope, in particular the 6B8 epitope that comprises at least the amino acid S at position 14, G at position 22, E or G at position 24, G at position 25, Y at position 10, D at position 41, and/or R at position 64 of the E2 protein. Preferably, the term 6B8 monoclonal antibody refers to a monoclonal antibody that comprises CDRs of the monoclonal antibody produced by a hybridoma deposited at CCTCC under the accession number CCTCC C2018120. Preferably, the term 6B8 monoclonal antibody refers to a monoclonal antibody that comprises a variable region of heavy chain (VH) complementarity-determining region 1 (CDR1) comprising the amino acid sequence set forth in SEQ ID NO: 1, a VH CDR2 comprising the amino acid sequence set forth in SEQ ID NO:2, a VH CDR3 comprising the amino acid sequence set forth in SEQ ID NO:3, a VL CDR1 comprising the amino acid sequence set forth in SEQ ID NO:4, a VL CDR2 comprising the amino acid sequence set forth in SEQ ID NO:5, and a VL CDR3 comprising the amino acid sequence set forth in SEQ ID NO:6. More preferably, the term 6B8 monoclonal antibody refers to a monoclonal antibody that comprises a heavy chain variable region (VH) having an amino acid sequence as set forth in SEQ ID NO: 7 and a light chain variable region (VL) having an amino acid sequence as set forth in SEQ ID NO: 8. More preferably the term 6B8 monoclonal antibody refers to the monoclonal antibody produced by a hybridoma deposited at CCTCC under the accession number CCTCC C2018120.

As used herein, “antibody” refers to immunoglobulins and immunoglobulin fragments, whether natural or partially or wholly synthetically, such as recombinantly, produced, including any fragment thereof containing at least a portion of the variable region of the immunoglobulin molecule that retains the binding specificity ability of the full-length immunoglobulin. Hence, an antibody includes any protein having a binding domain that is homologous or substantially homologous to an immunoglobulin antigen-binding domain (antibody combining site). Antibodies include antibody fragments. As used herein, the term antibody, thus, includes synthetic antibodies, recombinantly produced antibodies, multispecific antibodies (e.g., bispecific antibodies), human antibodies, non-human antibodies, humanized antibodies, chimeric antibodies, intrabodies, and antibody fragments. Antibodies provided herein include members of any immunoglobulin type (e.g., IgG, IgM, IgD, IgE, IgA and IgY), any class (e.g. IgG1, IgG2, IgG3, IgG4, IgA1 and IgA2) or subclass (e.g., IgG2a and IgG2b).

The term “variable region” as used herein means an immunoglobulin domain essentially consisting of four “framework regions” which are referred to in the art and hereinbelow as “framework region 1” or “FR1”; as “framework region 2” or “FR2”; as “framework region 3” or “FR3”; and as “framework region 4” or “FR4”, respectively; which framework regions are interrupted by three “complementarity determining regions” or “CDRs”, which are referred to in the art and hereinbelow as “complementarity determining region 1” or “CDR1”; as “complementarity determining region 2” or “CDR2”; and as “complementarity determining region 3” or “CDR3”, respectively. Thus, the general structure or sequence of an immunoglobulin variable region can be indicated as follows: FR1-CDR1-FR2-CDR2-FR3-CDR3-FR4. VH or VH refers to a heavy chain variable region, and VL or VL refers to a light chain variable region. Similarly, VH CDR1, VH CDR2 and VH CDR3 refer to CDR1, CDR2 and CDR3 of a heavy chain variable region, respectively. VL CDR1, VL CDR2 and VL CDR3 refer to CDR1, CDR2 and CDR3 of a light chain variable region, respectively.

As used herein, an “antibody fragment” or “antigen-binding fragment” of an antibody refers to any portion of a full-length antibody that is less than full length but contains at least a portion of the variable region of the antibody that binds antigen (e.g. one or more CDRs and/or one or more antibody combining sites) and thus retains the binding specificity, and at least a portion of the specific binding ability of the full-length antibody. Hence, an antigen-binding fragment refers to an antibody fragment that contains an antigen-binding portion that binds to the same antigen as the antibody from which the antibody fragment is derived. Antibody fragments include antibody derivatives produced by enzymatic treatment of full-length antibodies, as well as synthetically, e.g. recombinantly produced derivatives. An antibody fragment is included among antibodies. Examples of antibody fragments include, but are not limited to, Fab, Fab′, F(ab′)2, single-chain Fv (scFv), Fv, dsFv, diabody, Fd and Fd′ fragments and other fragments, including modified fragments (see, for example, Methods in Molecular Biology, Vol 207: Recombinant Antibodies for Cancer Therapy Methods and Protocols (2003); Chapter 1; p 3-25, Kipriyanov). The fragment can include multiple chains linked together, such as by disulfide bridges and/or by peptide linkers. An antigen-binding fragment includes any antibody fragment that when inserted into an antibody framework (such as by replacing a corresponding region) results in an antibody that immunospecifically binds (i.e. exhibits Ka of at least or at least about 10⁷-10⁸ M⁻¹) to the antigen.

The term “antigen-binding fragment of the 6B8 monoclonal antibody” refers to a fragment of the 6B8 monoclonal antibody or at least encodes for an amino acid sequence that specifically recognizes the 6B8 epitope, in particular the 6B8 epitope that comprises at least the amino acid S at position 14, G at position 22, E or G at position 24, G at position 25, Y at position 10, D at position 41, and/or R at position 64 of the E2 protein. The term further encompasses an amino acid fragment coding for a VH CDR1 comprising the amino acid sequence set forth in SEQ ID NO:1, a VH CDR2 comprising the amino acid sequence set forth in SEQ ID NO:2, a VH CDR3 comprising the amino acid sequence set forth in SEQ ID NO:3, and/or a VL CDR1 comprising the amino acid sequence set forth in SEQ ID NO: 4, a VL CDR2 comprising the amino acid sequence set forth in SEQ ID NO:5, and a VL CDR3 comprising the amino acid sequence set forth in SEQ ID NO:6. Moreover, the term also encompasses an amino acid fragment that comprises a heavy chain variable region (VH) having an amino acid sequence as set forth in SEQ ID NO: 7 and/or a light chain variable region (VL) having an amino acid sequence as set forth in SEQ ID NO: 8. More preferably the term encompasses an amino acid fragment encoded by the monoclonal antibody produced by a hybridoma deposited at CCTCC under the accession number CCTCC C2018120, which amino acid fragment specifically binds to the 6B8 epitope.

In one aspect, the replacement results in a mutated 6B8 epitope in the recombinant CSFV E2 protein, for example, a mutated 6B8 epitope to which the binding of the 6B8 monoclonal antibody or the antigen-binding fragment thereof is specifically inhibited. Herein, the use of “replacement/replaced/replacing” means “swap/swapped/swapping” unless stated otherwise. In one aspect, such replacement results in that the binding of the recombinant CSFV E2 protein to the 6B8 monoclonal antibody or the antigen-binding fragment thereof is specifically inhibited as compared with a corresponding wildtype CSFV E2 protein.

The term “specifically inhibited” or “specific inhibition” means that the 6B8 antibody binds with an at least 2-times, preferably 5-times, more preferably 10-times and even more preferably 50-times lower affinity to the mutated 6B8 epitope in comparison to the unmodified 6B8 epitope, in particular to the unmodified 6B8 epitope having the amino acid S at position 14, G at position 22, E or G at position 24, G at position 25, Y at position 10, D at position 41, R at position 64 of the E2 protein, or combinations thereof. “Affinity” is the interaction between a single antigen-binding site on an antibody molecule and a single epitope. It is expressed by the association constant KA=kass/kdiss, or the dissociation constant KD=k_{diss}/kₐₛₛ. More preferably, the term “specifically inhibited” or “specific inhibition” means that the 6B8 monoclonal antibody, in particular the monoclonal antibody produced by a hybridoma deposited at CCTCC under the accession number CCTCC C2018120 does not detectably bind to the mutated 6B8 epitope according the invention in an immunofluorescence assay, in a western blot assay, in an EIA (enzyme immunoassay), in an ELISA (enzyme linked immunosorbent assay) assay or a double competitive ELISA assay, or in a Dot blot assay.

The term “pestivirus other than CSFV” as used herein refers to all other viruses in the genus Pestivirus within the family Flaviviridae other than CSFV.

In one aspect, the E2 protein of the “pestivirus other than CSFV” cannot be specifically recognized and/or bound by the 6B8 monoclonal antibody or the antigen-binding fragment thereof.

The pestivirus other than CSFV includes but is not limited to the species selected from the group consisting of bovine viral diarrahea viruses, border disease viruses, and atypical pestiviruses; preferably is selected from bovine viral diarrahea viruses comprising BVDV-2; border disease viruses comprising Switzerland, BDV-1, BDV-2, BDV-3, BDV-4, BDV-5, BDV-6, Chamois, Italy, Turkey, and Tunisian_sheep_virus (TSV); and atypical pestiviruses comprising Giraffe pestivirus, BVDV-3, Pronghorn antelope, Bungowannah virus and Norway rat pestivirus. Some specific strains of the pestivirus other than CSFV include but are not limited to Giraffe pestivirus PG2 strain, BDV-2 Reindeer V60, Norway rat pestivirus isolate NrPV/NYC-D23, Tunisian sheep virus 9552 (TSV9552), Bungowannah 6778, or BVDV3 Hobi-like Th/04 KhonKaen.

In one aspect, the “pestivirus other than CSFV” is the Giraffe pestivirus PG2 strain, and the examplinary E2 protein thereof comprises an amino acid sequence of SEQ ID NO:21. In one aspect, the “pestivirus other than CSFV” is the BDV-2 Reindeer V60 strain, and the examplinary E2 protein thereof comprises an amino acid sequence of SEQ ID NO:22. In one aspect, the “pestivirus other than CSFV” is the Norway rat pestivirus isolate NrPV/NYC-D23, and the examplinary E2 protein thereof comprises an amino acid sequence of SEQ ID NO:23. In one aspect, the “pestivirus other than CSFV” is the Bungowannah virus 6778, and the examplinary E2 protein thereof comprises an amino acid sequence of SEQ ID NO:24. In one aspect, the “pestivirus other than CSFV” is the TSV9552 strain, and the examplinary E2 protein thereof comprises an amino acid sequence of SEQ ID NO:25. In one aspect, the “pestivirus other than CSFV” is the BVDV3 Hobi-like Th/04 KhonKaen strain, and the examplinary E2 protein thereof comprises an amino acid sequence of SEQ ID NO:26.

The CSFV E2 protein contains four antigenic domains, A, B, C and D domains, and all these domains are located at the N-terminus of the E2 protein. The four domains constitute two independent antigenic units, one is the unit of B/C domains and the other comprises A/D domains. The B/C domain is from amino acid position 1 to around position 111 and D/A domain is located from amino acid position 77 to around position 177. The numbering is determined based on the position numbers of the amino acid sequence of QZ07 CSFV E2 protein (SEQ ID NO: 10) in an exemplary manner. The B/C domain covers most key amino acids defining the 6B8 epitope of the CSFV E2 protein.

In one aspect, the fragment comprising at least one of the amino acids defining the 6B8 epitope of the CSFV E2 protein is the B/C domain or a fragment thereof of the CSFV E2 protein. In one aspect, the B/C domain of the CSFV E2 protein comprises the sequence from amino acid position 1 to around amino acid position 111 of the CSFV E2 protein.

In one aspect, the sequence of amino acid position 11 to amino acid position 38 of the CSFV E2 protein is replaced by a corresponding sequence of the E2 protein from a pestivirus other than CSFV. In one aspect, the sequence of amino acid position 11 to amino acid position 39 of the CSFV E2 protein is replaced by a corresponding sequence of the E2 protein from a pestivirus other than CSFV. In one aspect, the sequence of amino acid position 11 to amino acid position 56 of the CSFV E2 protein is replaced by a corresponding sequence of the E2 protein from a pestivirus other than CSFV. In one aspect, the sequence of amino acid position 11 to amino acid position 80 of the CSFV E2 protein is replaced by a corresponding sequence of the E2 protein from a pestivirus other than CSFV. In one aspect, the sequence of amino acid position 11 to amino acid position 90 of the CSFV E2 protein is replaced by a corresponding sequence of the E2 protein from a pestivirus other than CSFV. In one aspect, the sequence of amino acid position 11 to amino acid position 109 of the CSFV E2 protein is replaced by a corresponding sequence of the E2 protein from a pestivirus other than CSFV. In one aspect, the sequence of amino acid position 11 to amino acid position 110 of the CSFV E2 protein is replaced by a corresponding sequence of the E2 protein from a pestivirus other than CSFV.

“A corresponding sequence of the E2 protein from a pestivirus other than CSFV” means the sequence in the E2 protein from a pestivirus other than CSFV that aligns with the sequence in the CSFV E2 protein to be replaced in a sequence alignment.

Methods for aligning two amino acid or nucleotide sequences are well known in the art. Sequence analysis softwares are often used to perform sequence alignment. For example, sequence alignment can be done by using the BLAST program at NCBI database.

For example, the sequence of amino acid position 11 to position 110 of SEQ ID NO:10 (QZ07 CSFV E2 protein) aligns with the sequence of amino acid position 10 to position 111 of SEQ ID NO:24 (Bungowannah E2 protein) in a pairwise sequence alignment, then, the sequence of amino acid position 10 to position 111 (102 aa) of SEQ ID NO:24 (Bungowannah E2 protein) can be used to replace the sequence of amino acid position 11 to position 110 (100 aa) of SEQ ID NO:10 (QZ07 CSFV E2 protein).

In a preferred aspect of the invention, the recombinant CSFV E2 protein as disclosed herein is immunogenic and preferably confers protective immunity against CSFV. It is reported that CSFV E2 protein containing merely one of above mentioned 4 antigenic domains (A, B, C and D domains) remained immunogenic and can protect pigs from infectious CSFV challenge. Therefore, in a preferred aspect of the invention, the recombinant CSFV E2 protein as described herein retains at least one, preferably at least one of the antigenic domains as described above. Preferably, the recombinant CSFV E2 protein of the invention can confer protective immunity against CSFV. In one aspect, the replacement can be introduced without substantially affecting the protective immunogenicity of the recombinant CSFV E2 protein against CSFV.

In one aspect of the invention, the 6B8 epitope of the CSFV E2 protein specifically recognized by the 6B8 monoclonal antibody is defined at least by the amino acid residue at position 14, position 22, position 24, positions 24 and 25 (“24/25”), position 10, position 41 and/or position 64 of the CSFV E2 protein.

In one aspect of the invention, the 6B8 epitope of the CSFV E2 protein specifically recognized by the 6B8 monoclonal antibody is defined at least by the amino acid residue S14, G22, E24, E24/G25, Y10, D41 and/or R64 of the CSFV E2 protein, such as for isolates QZ07, GD18 or GD191. In one aspect of the invention, the 6B8 epitope of the CSFV E2 protein specifically recognized by the 6B8 monoclonal antibody is defined at least by the amino acid residue S14, G22, G24, G24/G25, Y10, D41 and/or R64 of the E2 protein, such as for C-strain.

In one aspect of the invention, as compared with the wildtype CSFV E2 protein, the recombinant CSFV E2 protein according to the invention comprises at least one amino acid residue at amino acid positions defining the 6B8 epitope of the CSFV E2 protein (e.g., position 14, position 22, position 24, positions 24 and 25 (“24/25”), position 10, position 41 and/or position 64) which specifically inhbit the binding of 6B8 monoclonal antibody to the CSFV E2 protein.

The inventors of the invention surprisingly found that the replacement of a fragment comprising at least one of the amino acids defining the 6B8 epitope of a CSFV E2 protein by a corresponding fragment of the E2 protein from a pestivirus other than CSFV and the incorporation of amino acid mutations at position 10, 14, 22, 24, 25, 24/25, 41 and/or 64 in a recombinant CSFV E2 protein did not substantially affect the protective immunogenicity or efficacy of the recombinant CSFV E2 protein against CSF. Moreover, the mutations at amino acid position 10, 14, 22, 24, 25, 24/25, 41, and/or 64 of the recombinant CSFV E2 protein, in which a fragment comprising at least one of the amino acids defining the 6B8 epitope of the CSFV E2 protein is replaced by a corresponding fragment of the E2 protein from a pestivirus other than CSFV further ensure the differentiating ability or increase the DIVA reliability of such recombinant CSFV E2 protein with respect to its DIVA feature (inhibition of the binding of an 6B8 antibody that normally recognizes and binds to the 6B8 epitope).

In some embodiments, said at least one amino acid residue is introduced by the replacement mentioned above. For example, in one aspect of the invention, the “corresponding fragment of the E2 protein from a pestivirus other than CSFV” comprises at least one amino acid residue at amino acid positions defining the 6B8 epitope of the CSFV E2 protein (e.g., position 14, position 22, position 24, positions 24 and 25 (“24/25”), position 10, position 41 and/or position 64) which specifically inhbit the binding of 6B8 monoclonal antibody to the CSFV E2 protein; or the “corresponding fragment of the E2 protein from a pestivirus other than CSFV” is engineered (mutated) to comprise at least one amino acid residue at amino acid positions defining the 6B8 epitope of the CSFV E2 protein (e.g., position 14, position 22, position 24, positions 24 and 25 (“24/25”), position 10, position 41 and/or position 64) which specifically inhbit the binding of 6B8 monoclonal antibody to the CSFV E2 protein when the wildtype sequence does not include said amino acids.

In some embodiments, in addition to the replacement with the “corresponding fragment of the E2 protein from a pestivirus other than CSFV”, at least one amino acid residue which specifically inhbit the binding of 6B8 monoclonal antibody is additionally introduced in the recombinant CSFV E2 protein. For example, the at least one amino acid residue which specifically inhbit the binding of 6B8 monoclonal antibody is located outside the fragment to be replaced.

In one aspect of the invention, the amino acid at position 24 of the recombinant CSFV E2 protein is R, D or I, with R being preferred, the amino acid at positions 24 and 25 of the recombinant CSFV E2 protein is R, D or I, with R being preferred, and D, K, L, N, R, T, V, E or P, with D, K, L, N, R, T and V being preferred, respectively, the amino acid at position 14 of the recombinant CSFV E2 protein is K, A, E, Q or R, with K being preferred, the amino acid at position 22 of the recombinant CSFV E2 protein is A, R, Q, E, D, N, K, L, P, T, V or S, with A, Q, D, E, N and S being preferred, the amino acid at position 10 of the recombinant CSFV E2 protein is A or P, the amino acid at position 41 of the recombinant CSFV E2 protein is A, N or E, and/or the amino acid at position 64 of the recombinant CSFV E2 protein is A, S, E, D, G, H, T, L, P, K or W, with A, K, and W being preferred. These amino acids, individually or in combination, have been demonstrated as specifically inhibiting the binding of 6B8 monoclonal antibody to the CSFV E2 protein. In one aspect of the invention, the corresponding fragment of the E2 protein from a pestivirus other than CSFV is mutated to introduce one or more said amino acids at corresponding positions.

In one aspect of the invention, the amino acid at position 24 of the recombinant CSFV E2 protein is R, D or I, with R being preferred.

In one aspect of the invention the amino acid at positions 24 and 25 of the recombinant CSFV E2 protein is R, D or I, with R being preferred, and D, K, L, N, R, T, V, E or P, with D, K, L, N, R, T and V being preferred, respectively.

In one aspect the amino acid at position 14 of the recombinant CSFV E2 protein is K, A, E, Q or R, with K being preferred.

In one aspect the amino acid at position 22 of the recombinant CSFV E2 protein is A, R, Q, E, D, N, K, L, P, T, V or S, with A, Q, D, E, N and S being preferred.

In one aspect the amino acid at position 10 of the recombinant CSFV E2 protein is A or P.

In one aspect the amino acid at position 41 of the recombinant CSFV E2 protein is A, N or E.

In one aspect the amino acid at position 64 of the recombinant CSFV E2 protein is A, S, E, D, G, H, T, L, P, K or W, with A, K, and W being preferred.

In one aspect of the invention, the corresponding fragment of the E2 protein from a pestivirus other than CSFV is mutated to introduce one or more said amino acids at corresponding positions. For example, in one aspect of the invention, the recombinant CSFV E2 protein according to the invention comprises mutations at the amino acid positions of the E2 protein as defined in the following tables 1 to 7. For example, the second line in table 1 means that the recombinant CSFV E2 protein according to the invention comprises a mutation at amino acid position 10 (“position 10”) of the E2 protein, and the third line in table 1 means that the recombinant CSFV E2 protein according to the invention comprises mutations at amino acid positions 10 and 14 of the E2 protein, etc.

TABLE 1

| posi- | posi- | posi- | posi- | posi- | posi- | posi- | posi- |
|---|---|---|---|---|---|---|---|
| tion | tion | tion | tion | tion | tion | tion | tion |
| 10 | 14 | 22 | 24 | 25 | 24/25 | 41 | 64 |
| x | | | | | | | |
| x | x | | | | | | |
| x | | x | | | | | |
| x | | | x | | | | |
| x | | | | x | x | | |
| x | | | | | | x | |
| x | | | | | | | x |
| x | x | x | | | | | |
| x | x | | x | | | | |
| x | x | | | x | x | | |
| x | x | | | | | x | |
| x | x | | | | | | x |
| x | | x | x | | | | |
| x | | x | | x | x | | |
| x | | x | | | | x | |
| x | | x | | | | | x |
| x | | | x | x | x | | |
| x | | | x | | | x | |
| x | | | x | | | | x |
| x | | | | x | x | x | |
| x | | | | x | x | | x |
| x | | | | | | x | x |
| x | x | x | x | | | | |
| x | x | x | | x | x | | |
| x | x | x | | | | x | |
| x | x | x | | | | | x |
| x | x | | x | x | x | | |
| x | x | | x | | | x | |
| x | x | | x | | | | x |
| x | x | | | x | x | x | |
| x | x | | | x | x | | x |
| x | x | | | | | x | x |
| x | | x | x | x | x | | |
| x | | x | x | | | x | |
| x | | x | x | | | | x |
| x | | | x | x | x | x | |
| x | | | x | x | x | | x |
| x | | | | x | x | x | x |
| x | x | x | x | x | x | | |
| x | x | x | x | | | x | |
| x | x | x | x | | | | x |
| x | | x | x | x | x | x | |
| x | | x | x | x | x | | |
| x | x | x | x | x | x | x | |
| x | x | x | x | x | x | | x |
| x | x | x | x | x | x | x | x |

TABLE 2

| posi- | posi- | posi- | posi- | posi- | posi- | posi- | posi- |
|---|---|---|---|---|---|---|---|
| tion | tion | tion | tion | tion | tion | tion | tion |
| 41 | 10 | 14 | 22 | 24 | 25 | 24/25 | 64 |
| x | | | | | | | |
| x | x | | | | | | |
| x | | x | | | | | |
| x | | | x | | | | |
| x | | | | x | | | |
| x | | | | | x | x | |
| x | | | | | | | x |
| x | x | x | | | | | |
| x | x | | x | | | | |
| x | x | | | x | | | |
| x | x | | | | x | x | |
| x | x | | | | | | x |
| x | | x | x | | | | |
| x | | x | | x | | | |
| x | | x | | | x | x | |
| x | | x | | | | | x |
| x | | | x | x | | | |
| x | | | x | | x | x | |
| x | | | x | | | | x |
| x | | | | x | x | x | |
| x | | | | x | | | x |
| x | | | | | x | x | x |
| x | x | x | x | | | | |
| x | x | x | | x | | | |
| x | x | x | | | x | x | |
| x | x | x | | | | | x |
| x | x | | x | x | | | |
| x | x | | x | | x | x | |
| x | x | | x | | | | x |
| x | x | | | x | x | x | |
| x | x | | | x | | | x |
| x | x | | | | x | x | x |
| x | | x | x | x | | | |
| x | | x | x | | x | x | |
| x | | x | x | | | | x |
| x | | | x | x | x | x | |
| x | | | x | x | | | x |
| x | | | | x | x | x | x |
| x | x | x | x | x | | | |
| x | x | x | x | | x | x | |
| x | x | x | x | | | | x |
| x | | x | x | x | x | x | |
| x | | x | x | x | | | x |
| x | x | x | x | x | x | x | |
| x | x | x | x | x | | | x |
| x | x | x | x | x | x | x | x |

TABLE 3

| posi- | posi- | posi- | posi- | posi- | posi- | posi- | posi- |
|---|---|---|---|---|---|---|---|
| tion | tion | tion | tion | tion | tion | tion | tion |
| 64 | 10 | 14 | 22 | 24 | 25 | 24/25 | 41 |
| x | | | | | | | |
| x | x | | | | | | |
| x | | x | | | | | |
| x | | | x | | | | |
| x | | | | x | | | |
| x | | | | | x | x | |
| x | | | | | | | x |
| x | x | x | | | | | |
| x | x | | x | | | | |
| x | x | | | x | | | |
| x | x | | | | x | x | |
| x | x | | | | | | x |
| x | | x | x | | | | |
| x | | x | | x | | | |
| x | | x | | | x | x | |
| x | | x | | | | | x |
| x | | | x | x | | | |
| x | | | x | | x | x | |
| x | | | x | | | | x |
| x | | | | x | x | x | |
| x | | | | x | | | x |
| x | | | | | x | x | x |
| x | x | x | x | | | | |
| x | x | x | | x | | | |
| x | x | x | | | x | x | |
| x | x | x | | | | | x |
| x | x | | x | x | | | |
| x | x | | x | | x | x | |
| x | x | | x | | | | x |
| x | x | | | x | x | x | |
| x | x | | | x | | | x |
| x | x | | | x | x | x | |
| x | x | | | x | | | |
| x | x | | | | x | x | x |
| x | | x | x | x | | | |
| x | | x | x | | x | x | |
| x | | x | x | | | | x |
| x | | | x | x | x | x | |
| x | | | x | x | | | x |
| x | | | | x | x | x | x |
| x | x | x | x | x | | | |
| x | x | x | x | | x | x | |
| x | x | x | x | | | | x |
| x | | x | x | x | x | x | |
| x | | x | x | x | | | x |
| x | x | x | x | x | x | x | |
| x | x | x | x | x | | | x |
| x | x | x | x | x | x | x | x |

TABLE 4

| posi- | posi- | posi- | posi- | posi- | posi- | posi- | posi- |
|---|---|---|---|---|---|---|---|
| tion | tion | tion | tion | tion | tion | tion | tion |
| 14 | 10 | 22 | 24 | 25 | 24/25 | 41 | 64 |
| x | | | | | | | |
| x | x | | | | | | |
| x | | x | | | | | |
| x | | | x | | | | |
| x | | | | x | x | | |
| x | | | | | | x | |
| x | | | | | | | x |
| x | x | x | | | | | |
| x | x | | x | | | | |
| x | x | | | x | x | | |
| x | x | | | | | x | |
| x | x | | | | | | x |
| x | | x | x | | | | |
| x | | x | | x | x | | |
| x | | x | | | | x | |
| x | | x | | | | | x |
| x | | | x | x | x | | |
| x | | | x | | | x | |
| x | | | x | | | | x |
| x | | | | x | x | x | |
| x | | | | x | x | | x |
| x | | | | | | x | x |
| x | x | x | x | | | | |
| x | x | x | | x | x | | |
| x | x | x | | | | x | |
| x | x | x | | | | | x |
| x | x | | x | x | x | | |
| x | x | | x | | | x | |
| x | x | | x | | | | x |
| x | x | | | x | x | x | |
| x | x | | | x | x | | x |
| x | x | | | | | x | x |
| x | | x | x | x | x | | |
| x | | x | x | | | x | |
| x | | x | x | | | | x |
| x | | | x | x | x | x | |
| x | | | x | x | x | | x |
| x | | | | x | x | x | x |
| x | x | x | x | x | x | | |
| x | x | x | x | | | x | |
| x | x | x | x | | | | x |
| x | | x | x | x | x | x | |
| x | | x | x | x | x | | x |
| x | x | x | x | x | x | x | |
| x | x | x | x | x | x | | x |
| x | x | x | x | x | x | x | x |

TABLE 5

| posi- | posi- | posi- | posi- | posi- | posi- | posi- | posi- |
|---|---|---|---|---|---|---|---|
| tion | tion | tion | tion | tion | tion | tion | tion |
| 22 | 10 | 14 | 24 | 25 | 24/25 | 41 | 64 |
| x | | | | | | | |
| x | x | | | | | | |
| x | | x | | | | | |
| x | | | x | | | | |
| x | | | | x | x | | |
| x | | | | | | x | |
| x | | | | | | | x |
| x | x | x | | | | | |
| x | x | | x | | | | |
| x | x | | | x | x | | |
| x | x | | | | | x | |
| x | x | | | | | | x |
| x | | x | x | | | | |
| x | | x | | x | x | | |
| x | | x | | | | x | |
| x | | x | | | | | x |
| x | | | x | x | x | | |
| x | | | x | | | x | |
| x | | | x | | | | x |
| x | | | | x | x | x | |
| x | | | | x | x | | x |
| x | | | | | | x | x |
| x | x | x | x | | | | |
| x | x | x | | x | x | | |
| x | x | x | | | | x | |
| x | x | x | | | | | x |
| x | x | | x | x | x | | |
| x | x | | x | | | x | |
| x | x | | x | | | | x |
| x | x | | | x | x | x | |
| x | x | | | x | x | | x |
| x | x | | | | | x | x |
| x | | x | x | x | x | | |
| x | | x | x | | | x | |
| x | | x | x | | | | x |
| x | | | x | x | x | x | |
| x | | | x | x | x | | x |
| x | | | | x | x | x | x |
| x | x | x | x | x | x | | |
| x | x | x | x | | | x | |
| x | x | x | x | | | | x |
| x | | x | x | x | x | x | |
| x | | x | x | x | x | | x |
| x | x | x | x | x | x | x | |
| x | x | x | x | x | x | | x |
| x | x | x | x | x | x | x | x |

TABLE 6

| posi- | posi- | posi- | posi- | posi- | posi- | posi- | posi- |
|---|---|---|---|---|---|---|---|
| tion | tion | tion | tion | tion | tion | tion | tion |
| 24 | 10 | 14 | 22 | 25 | 24/25 | 41 | 64 |
| x | | | | | | | |
| x | x | | | | | | |
| x | | x | | | | | |
| x | | | x | | | | |
| x | | | | x | x | | |
| x | | | | | | x | |
| x | | | | | | | x |
| x | x | x | | | | | |
| x | x | | x | | | | |
| x | x | | | x | x | | |
| x | x | | | | | x | |
| x | x | | | | | | x |
| x | | x | x | | | | |
| x | | x | | x | x | | |
| x | | x | | | | x | |
| x | | x | | | | | x |
| x | | | x | x | x | | |
| x | | | x | | | x | |
| x | | | x | | | | x |
| x | | | | x | x | x | |
| x | | | | x | x | | x |
| x | | | | | | x | x |
| x | x | x | x | | | | |
| x | x | x | | x | x | | |
| x | x | x | | | | x | |
| x | x | x | | | | | x |
| x | x | | x | x | x | | |
| x | x | | x | | | x | |
| x | x | | x | | | | x |
| x | x | | | x | x | x | |
| x | x | | | x | x | | x |
| x | x | | | | | x | x |
| x | | x | x | x | x | | |
| x | | x | x | | | x | |
| x | | x | x | | | | x |
| x | | | x | x | x | x | |
| x | | | x | x | x | | x |
| x | | | | x | x | x | x |
| x | x | x | x | x | x | | |
| x | x | x | x | | | x | |
| x | x | x | x | | | | x |
| x | | x | x | x | x | x | |
| x | | x | x | x | x | | x |
| x | x | x | x | x | x | x | |
| x | x | x | x | x | x | | x |
| x | x | x | x | x | x | x | x |

TABLE 7A

| position | position | position | position | position | position | position |
|---|---|---|---|---|---|---|
| 25 | 10 | 14 | 22 | 24 | 41 | 64 |
| x | | | | | | |
| x | x | | | | | |
| x | | x | | | | |
| x | | | x | | | |
| x | | | | x | | |
| x | | | | | x | |
| x | | | | | | x |
| x | x | x | | | | |
| x | x | | x | | | |
| x | x | | | x | | |
| x | x | | | | x | |
| x | x | | | | | x |
| x | | x | x | | | |
| x | | x | | x | | |
| x | | x | | | x | |
| x | | x | | | | x |
| x | | | x | x | | |
| x | | | x | | x | |
| x | | | x | | | x |
| x | | | | x | x | |
| x | | | | x | | x |
| x | | | | | x | x |
| x | x | x | x | | | |
| x | x | x | | x | | |
| x | x | x | | | x | |
| x | x | x | | | | x |
| x | x | | x | x | | |
| x | x | | x | | x | |
| x | x | | x | | | x |
| x | x | | | x | x | |
| x | x | | | x | | x |
| x | x | | | | x | x |
| x | | x | x | x | | |
| x | | x | x | | x | |
| x | | x | x | | | x |
| x | | | x | x | x | |
| x | | | x | x | | x |
| x | | | | x | x | x |
| x | x | x | x | x | | |
| x | x | x | x | | x | |
| x | x | x | x | | | x |
| x | | x | x | x | x | |
| x | | x | x | x | | x |
| x | x | x | x | x | x | |
| x | x | x | x | x | | x |
| x | x | x | x | x | x | x |

TABLE 7B

| positions | position | position | position | position | position | position |
|---|---|---|---|---|---|---|
| 24/25 | 10 | 14 | 22 | 24 | 41 | 64 |
| x | | | | | | |
| x | x | | | | | |
| x | | x | | | | |
| x | | | x | | | |
| x | | | | x | | |
| x | | | | | x | |
| x | | | | | | x |
| x | x | x | | | | |
| x | x | | x | | | |
| x | x | | | x | | |
| x | x | | | | x | |
| x | x | | | | | x |
| x | | x | x | | | |
| x | | x | | x | | |
| x | | x | | | x | |
| x | | x | | | | x |
| x | | | x | x | | |
| x | | | x | | x | |
| x | | | x | | | x |
| x | | | | x | x | |
| x | | | | x | | x |
| x | | | | | x | x |
| x | x | x | x | | | |
| x | x | x | | x | | |
| x | x | x | | | x | |
| x | x | x | | | | x |
| x | x | | x | x | | |
| x | x | | x | | x | |
| x | x | | x | | | x |
| x | x | | | x | x | |
| x | x | | | x | | x |
| x | x | | | | x | x |
| x | | x | x | x | | |
| x | | x | x | | x | |
| x | | x | x | | | x |
| x | | | x | x | x | |
| x | | | x | x | | x |
| x | | | | x | x | x |
| x | x | x | x | x | | |
| x | x | x | x | | x | |
| x | x | x | x | | | x |
| x | | x | x | x | x | |
| x | | x | x | x | | x |
| x | x | x | x | x | x | |
| x | x | x | x | x | | x |
| x | x | x | x | x | x | x |

In one aspect of the invention, the amino acid at position 24 of the recombinant CSFV E2 protein is R, D or I, with R being preferred, the amino acid at positions 24 and 25 of the recombinant CSFV E2 protein is R, D or I, with R being preferred, and D, K, L, N, R, T, V, E or P, with D, K, L, N, R, T and V being preferred, respectively, the amino acid at position 14 of the recombinant CSFV E2 protein is K, A, E, Q or R, with K being preferred, and the amino acid at position 22 of the recombinant CSFV E2 protein is A, R, Q, E, D, N, K, L, P, T, V or S, with A, Q, D, E, N and S being preferred.

In one aspect of the invention, the amino acid at position 24 of the recombinant CSFV E2 protein is R, the amino acid at positions 25 of the recombinant CSFV E2 protein is D, the amino acid at position 14 of the recombinant CSFV E2 protein is K, and the amino acid at position 22 of the recombinant CSFV E2 protein is A (hereinafter referred to “KARD mutation”).

In one aspect of the invention, the recombinant CSFV E2 protein comprises an amino acid sequence selected from the group consisting of SEQ ID NOs: 27-48 and 71-74.

In one aspect of the invention, in order to obtain a soluble recombinant CSFV E2 protein, the recombinant CSFV E2 protein according to the invention may be truncated to remove the transmembrane domain. For example, the last about 40 amino acids (e.g., 42 or 43 amino acids) of the C-terminus of the intact CSFV E2 protein according to the invention may be deleted.

In one aspect of the invention, in order to obtain a secreted format of the recombinant E2 protein according to the invention, a signal peptide can be added to the N-terminus of the E2 protein. For example, the last about 20 amino acids, in particular the last 16 amino acids (e.g., for C-strain) or 21-23 amino acids (e.g., for GD18 or QZ07), from El protein can be added to the N-terminus of the recombinant E2 protein according to the invention. In one aspect, the signal peptide may comprises an amino acid sequence selected from SEQ ID NOs: 13-15. A person skilled in the art would acknowledge that other signal peptide allowing secret expression can also be applied in the present invention.

In one aspect of the invention, the recombinant CSFV E2 protein may be truncated to remove the transmembrane domain and a signal peptide can be added to the N-terminus of the recombinant CSFV E2 protein, so as to obtain a soluble and secreted recombinant CSFV E2 protein, for example, the last 43 amino acids of the intact recombinant CSFV E2 protein may be deleted and the last 16 amino acids or 21-23 amino acids from El protein can be added to the N-terminus of the recombinant CSFV E2 protein.

In one aspect of the invention, the recombinant E2 protein may also comprises a fusion tag for identification and/or purification. Such tags are well known in the art, such as a His-tag or a FLAG-tag.

In one aspect of the invention, an immunoglobulin Fc fragment can be linked to the E2 protein. The term “immunoglobulin Fc fragment”, as used herein, refers to a protein that contains the heavy-chain constant region 2 (CH2) and the heavy-chain constant region 3 (CH3) of an immunoglobulin and, more particular, that does not contain the variable regions of the heavy and light chains, and the light-chain constant region 1 (CL1) of the immunoglobulin. It may further include the hinge region, or a portion of the hinge region, of the immunoglobulin (i.e., the hinge region at the heavy-chain constant region). Also, the immunoglobulin Fc fragment may contain a part of the heavy-chain constant region 1 (CH1). It is understood that the term “immunoglobulin Fc fragment”, as used herein, is equivalent to “Fc fragment”.

The herein used term “linked to” in particular refers to any means for connecting, within a polypeptide, an immunoglobulin Fc fragment to the C-terminus or N-terminus of the recombinant CSFV E2 protein. Example of linking means may include direct linkage of the immunoglobulin Fc fragment to the CSFV E2 protein by covalent bonding. In one aspect, the Fc fragment can be linked to the C terminus of the CSFV E2 protein.

In one aspect, the immunoglobulin Fc fragment can be linked to the CSFV E2 protein via a peptide linker. The term “peptide linker” as used herein refers to a peptide comprising one or more amino acid residues. More particular, the term “peptide linker” as used herein refers to a peptide capable of connecting two variable proteins and/or domains, e.g., the CSFV E2 protein and an immunoglobulin Fc fragment. The peptide linker mentioned herein is preferably an amino acid sequence being 3 to 20 amino acid residues in length. For example, the linker moiety may be a peptide linker being 1, 2, 3, 4, 5, 6, 7, 8, 9, or 10 amino acid residues in length. The exemplary sequences of the peptide linkers can be GGS, GSGGSG, GGGGS, GGGGSGGGGS. In one aspect, the amino acid sequence of the peptide linker is GGS (SEQ ID NO: 16).

In one aspect, the immunoglobulin Fc fragment is a swine IgG Fc fragment. In one aspect, the Fc fragment can be linked to the C terminus or N terminus of the E2 protein. In one aspect, the Fc fragment can be linked to the C terminus of the E2 protein via a peptide linker. In one aspect, the amino acid sequence of the swine Fc fragment is shown by SEQ ID NO: 20.

In one aspect of the invention, the recombinant CSFV E2 protein comprises or consists of one of the amino acid sequences selected from the group consisting of SEQ ID NOs: 49-70, 75-78 and 96-101. In one embodiment, the recombinant CSFV E2 protein comprises or consists of one of the amino acid sequences selected from the group consisting of SEQ ID NOs: 53, 68 and 75. In one embodiment, the recombinant CSFV E2 protein comprises or consists of one of the amino acid sequences selected from the group consisting of SEQ ID NOs: 53 and 75. In one embodiment, the recombinant CSFV E2 protein comprises or consists of the amino acid sequence of SEQ ID NO: 53. In one embodiment, the recombinant CSFV E2 protein comprises or consists of the amino acid sequence of SEQ ID NO: 68. In one embodiment, the recombinant CSFV E2 protein comprises or consists of the amino acid sequence of SEQ ID NO: 75.

It is understood by a person skilled in the art that any of Fc fragments described herein, in particular the Fc fragment having SEQ ID NO: 20 can be linked to any of recombinant CSFV E2 proteins disclosed herein in which a fragment comprising at least one of the amino acids defining the 6B8 epitope of the CSFV E2 protein is replaced by a corresponding fragment of the E2 protein from a pestivirus other than CSFV, including those having additional mutations at amino acid positions 10, 14, 22, 24, 25, 24/25, 41 and/or 64 as defined herein. The peptide linker can be any linker, in some embodiments the linker comprises or consists of an amino acid sequence selected from SEQ ID NOS: 16-19. In some embodiments, the peptide linker comprises or consists of an amino acid sequence of SEQ ID NO: 16.

In one aspect, the present invention provides a recombinant CSFV (classical swine fever virus) comprising the recombinant CSFV E2 protein of the invention. In one aspect of the invention, the recombinant CSFV is attenuated.

A person skilled in the art would acknowledge that the recombinant CSFV of the invention can be derived from various CSFV isolates, as the 6B8 epitope is evolutionarily conserved among different CSFV strains. In one aspect of the invention, the recombinant CSFV of the invention is derived from an isolate of genogroup 2.1. In one aspect of the invention, the recombinant CSFV is derived for example from the field strain GD18 or QZ07. In one aspect of the invention, the recombinant CSFV of the invention is derived from an isolate of genogroup 1. In one aspect of the invention, the recombinant CSFV is derived from the C-strain well known in the art.

In one aspect, the present invention provides a recombinant CSFV E2 protein, wherein the recombinant CSFV E2 protein comprises an amino acid sequence of the E2 protein of CSFV field strain QZ07 with a fragment comprising at least one of the amino acids defining the 6B6 epitope being replaced by a corresponding fragment of the E2 protein of BVDV3 Hobi-like Th/04 KhonKaen strain.

In one aspect, the sequence of amino acid position 11 to amino acid position 38 of the CSFV E2 protein is replaced by a corresponding sequence of the E2 protein from BVDV3 Hobi-like Th/04 KhonKaen strain, and the numbering of the amino acid position is determined based on the position numbers of the amino acid sequence of the E2 protein of CSFV field strain QZ07 shown by SEQ ID NO: 10.

In one aspect, the sequence of amino acid position 11 to amino acid position 39 of the CSFV E2 protein is replaced by a corresponding sequence of the E2 protein from BVDV3 Hobi-like Th/04 KhonKaen strain, and the numbering of the amino acid position is determined based on the position numbers of the amino acid sequence of the E2 protein of CSFV field strain QZ07 shown by SEQ ID NO: 10.

In one aspect, the sequence of amino acid position 11 to amino acid position 56 of the CSFV E2 protein is replaced by a corresponding sequence of the E2 protein from BVDV3 Hobi-like Th/04 KhonKaen strain, and the numbering of the amino acid position is determined based on the position numbers of the amino acid sequence of the E2 protein of CSFV field strain QZ07 shown by SEQ ID NO: 10.

In one aspect, the sequence of amino acid position 11 to amino acid position 80 of the CSFV E2 protein is replaced by a corresponding sequence of the E2 protein from BVDV3 Hobi-like Th/04 KhonKaen strain, and the numbering of the amino acid position is determined based on the position numbers of the amino acid sequence of the E2 protein of CSFV field strain QZ07 shown by SEQ ID NO: 10.

In one aspect, the sequence of amino acid position 11 to amino acid position 90 of the CSFV E2 protein is replaced by a corresponding sequence of the E2 protein from BVDV3 Hobi-like Th/04 KhonKaen strain, and the numbering of the amino acid position is determined based on the position numbers of the amino acid sequence of the E2 protein of CSFV field strain QZ07 shown by SEQ ID NO: 10.

In one aspect, the sequence of amino acid position 11 to amino acid position 109 of the CSFV E2 protein is replaced by a corresponding sequence of the E2 protein from BVDV3 Hobi-like Th/04 KhonKaen strain, and the numbering of the amino acid position is determined based on the position numbers of the amino acid sequence of the E2 protein of CSFV field strain QZ07 shown by SEQ ID NO: 10.

In one aspect, the sequence of amino acid position 11 to amino acid position 109 of the E2 protein of CSFV field strain QZ07 is replaced by a corresponding fragment of the E2 protein of BVDV3 Hobi-like Th/04 KhonKaen strain, and the numbering of the amino acid position is determined based on the position numbers of the amino acid sequence of the E2 protein of CSFV field strain QZ07 shown by SEQ ID NO: 10.

In some embodiments, compared to the corresponding fragment of the E2 protein of BVDV3 Hobi-like Th/04 KhonKaen strain, the recombinant CSFV E2 protein further comprises at least one amino acid mutation at positions 10, 14, 22, 24, 25, 24/25, 41 and/or 64. In some embodiments, compared to the corresponding fragment of the E2 protein of BVDV3 Hobi-like Th/04 KhonKaen strain, the recombinant CSFV E2 protein further comprises one single amino acid mutation at position 14, 24, 25 or 64 of the recombinant CSFV E2 protein.

In some embodiments, compared to the corresponding fragment of the E2 protein of BVDV3 Hobi-like Th/04 KhonKaen strain, the recombinant CSFV E2 protein further comprises the mutations at amino acid positions 10, 14, 22, 24, 25, 24/25, 41 and/or 64 as defined in Tables 1 to 7.

In some embodiments, the amino acid at position 14 of the recombinant CSFV E2 protein comprising a fragment of the E2 protein of BVDV3 Hobi-like Th/04 KhonKaen strain is K, A, E, Q or R; preferably the amino acid at position 14 of the recombinant CSFV E2 protein comprising a fragment of the E2 protein of BVDV3 Hobi-like Th/04 KhonKaen strain is K. In some embodiments, the amino acid at position 64 of the recombinant CSFV E2 protein comprising a fragment of the E2 protein of BVDV3 Hobi-like Th/04 KhonKaen strain is A, S, E, D, G, H, T, L, P, K or W; preferably the amino acid at position 64 of the recombinant CSFV E2 protein comprising a fragment of the E2 protein of BVDV3 Hobi-like Th/04 KhonKaen strain is K. In some embodiments, the amino acid at position 24 of the recombinant CSFV E2 protein comprising a fragment of the E2 protein of BVDV3 Hobi-like Th/04 KhonKaen strain is R, D or I; preferably the amino acid at position 24 of the recombinant CSFV E2 protein comprising a fragment of the E2 protein of BVDV3 Hobi-like Th/04 KhonKaen strain is R. In some embodiments, the amino acid at position 25 of the recombinant CSFV E2 protein comprising a fragment of the E2 protein of BVDV3 Hobi-like Th/04 KhonKaen strain is D, K, L, N, R, T, V, E or P; preferably the amino acid at position 25 of the recombinant CSFV E2 protein comprising a fragment of the E2 protein of BVDV3 Hobi-like Th/04 KhonKaen strain is D. In some embodiments, the amino acid at position 25 of the recombinant CSFV E2 protein comprising a fragment of the E2 protein of BVDV3 Hobi-like Th/04 KhonKaen strain is D, K, L, N, R, T, V, E or P; preferably the amino acid at position 25 of the recombinant CSFV E2 protein comprising a fragment of the E2 protein of BVDV3 Hobi-like Th/04 KhonKaen strain is N. In some embodiments, the amino acid at position 25 of the recombinant CSFV E2 protein comprising a fragment of the E2 protein of BVDV3 Hobi-like Th/04 KhonKaen strain is D, K, L, N, R, T, V, E or P; preferably the amino acid at position 25 of the recombinant CSFV E2 protein comprising a fragment of the E2 protein of BVDV3 Hobi-like Th/04 KhonKaen strain is R.

In some embodiments, an Fc fragment, such as a swine Fc fragment is linked to the recombinant CSFV E2 protein, in particular in which a fragment of the CSFV comprising at least one of the amino acids defining the 6B8 epitope is replaced by the corresponding fragment of the E2 protein of BVDV3 Hobi-like Th/04 KhonKaen strain. In some embodiments, an Fc fragment, such as a swine Fc fragment, is linked to the C terminus of the E2 protein. In some embodiments, an Fc fragment, such as a swine Fc fragment, is linked to the C terminus of the E2 protein via a peptide linker.

In some embodiments, the Fc fragment comprises or consists of an amino acid sequence of SEQ ID NO: 20. In some embodiments, the peptide linker comprises or consists of an amino acid sequence selected from SEQ ID NOs: 16-19. In some embodiments, the peptide linker comprises or consists of an amino acid sequence of SEQ ID NO: 16.

In some embodiments, the recombinant CSFV E2 protein comprises or consists of an amino acid sequence selected from the group consisting of SEQ ID NOs: 96-101. In some embodiments, the recombinant CSFV E2 protein comprises or consists of an amino acid sequence shown by SEQ ID NO: 96. In some embodiments, the recombinant CSFV E2 protein comprises or consists of an amino acid sequence shown by SEQ ID NO: 97. In some embodiments, the recombinant CSFV E2 protein comprises or consists of an amino acid sequence shown by SEQ ID NO: 98. In some embodiments, the recombinant CSFV E2 protein comprises or consists of an amino acid sequence shown by SEQ ID NO: 99. In some embodiments, the recombinant CSFV E2 protein comprises or consists of an amino acid sequence shown by SEQ ID NO: 100. In some embodiments, the recombinant CSFV E2 protein comprises or consists of an amino acid sequence shown by SEQ ID NO: 101.

It is well understood that in some embodiments the respective sequence of the BVDV3 Hobi-like Th/04 KhonKaen strain of the recombinant CSFV E2 proteins described above can be replaced by the corresponding sequences of another non CSFV pestivirus sequence, for example by any of the strains listed herein (e.g. Giraffe pestivirus PG2 strain, BDV-2 Reindeer V60, Norway rat pestivirus isolate NrPV/NYC-D23, Tunisian sheep virus 9552 (TSV9552), Bungowannah 6778). It is furthermore well understood that in some embodiments, such other recombinant CSFV E2 proteins may further comprise one or more mutations at amino acid positions 10, 14, 22, 24, 24/25, 41 and or 64 as defined in Tables 1 to 7. Moreover, it is also understood that in some embodiments, any such recombinant CSFV E2 proteins may be linked to an Fc fragment.

In one aspect, the present invention also provides an immunogenic composition comprising the recombinant CSFV E2 protein according to the present invention and/or the recombinant CSFV of the invention.

The term “immunogenic composition” as used herein refers to a composition that comprises at least one antigen, which elicits an immunological response in the host to which the immunogenic composition is administered. Such immunological response may be a cellular and/or antibody-mediated immune response to the immunogenic composition of the invention. The host is also described as “subject”. Preferably, any of the hosts or subjects described or mentioned herein is an animal.

Usually, an “immunological response” includes but is not limited to one or more of the following effects: the production or activation of antibodies, B cells, helper T cells, suppressor T cells, and/or cytotoxic T cells and/or gamma-delta T cells, directed specifically to an antigen or antigens included in the immunogenic composition of the invention. Preferably, the host will display either a protective immunological response or a therapeutically response.

A “protective immunological response” will be demonstrated by either a reduction or lack of clinical signs normally displayed by an infected host, a quicker recovery time and/or a lowered duration of infectivity or lowered pathogen titer in the tissues or body fluids or excretions of the infected host.

An “antigen” as used herein refers to, but is not limited to, components which elicit an immunological response in a host to an immunogenic composition or vaccine of interest comprising such antigen or an immunologically active component thereof.

In case where the host displays a protective immunological response such that resistance to new infection will be enhanced and/or the clinical severity of the disease reduced, the immunogenic composition is described as a “vaccine”.

In one aspect, the immunogenic composition of the present invention is a vaccine.

The term “vaccine” as understood herein is a vaccine for veterinary use comprising antigenic substances and is administered for the purpose of inducing a specific and active immunity against a disease provoked by a CSFV infection.

Preferably, the vaccine according to the invention is a subunit CSFV vaccine, comprising a recombinant CSFV E2 protein, preferably as described herein, eliciting a protective immune response in the host animal.

A vaccine may additionally comprise further components typical to pharmaceutical compositions.

Additional components to enhance the immune response are constituents commonly referred to as “adjuvants”, or ancillary molecules added to the vaccine or generated by the body after the respective induction by such additional components, like but not restricted to interferons, interleukins or growth factors. “Adjuvants” as used herein, can include aluminum hydroxide and aluminum phosphate, saponins e.g., Quil A, QS-21 (Cambridge Biotech Inc., Cambridge MA), GPI-0100 (Galenica Pharmaceuticals, Inc., Birmingham, AL), water-in-oil emulsion, oil-in-water emulsion, water-in-oil-in-water emulsion. The emulsion can be based in particular on light liquid paraffin oil (European Pharmacopea type); isoprenoid oil such as squalane or squalene; oil resulting from the oligomerization of alkenes, in particular of isobutene or decene; esters of acids or of alcohols containing a linear alkyl group, more particularly plant oils, ethyl oleate, propylene glycol di-(caprylate/caprate), glyceryl tri-(caprylate/caprate) or propylene glycol dioleate; esters of branched fatty acids or alcohols, in particular isostearic acid esters. The oil is used in combination with emulsifiers to form the emulsion. The emulsifiers are preferably nonionic surfactants, in particular esters of sorbitan, of mannide (e.g. anhydromannitol oleate), of glycol, of polyglycerol, of propylene glycol and of oleic, isostearic, ricinoleic or hydroxystearic acid, which are optionally ethoxylated, and polyoxypropylene-polyoxyethylene copolymer blocks, in particular the Pluronic products, especially L121. See Hunter et al., The Theory and Practical Application of Adjuvants (Ed. Stewart-Tull, D. E. S.), JohnWiley and Sons, NY, pp 51-94 (1995) and Todd et al., Vaccine 15:564-570 (1997). Exemplary adjuvants are the SPT emulsion described on page 147 of “Vaccine Design, The Subunit and Adjuvant Approach” edited by M. Powell and M. Newman, Plenum Press, 1995, and the emulsion MF59 described on page 183 of this same book.

A further instance of an adjuvant is a compound chosen from the polymers of acrylic or methacrylic acid and the copolymers of maleic anhydride and alkenyl derivative. Advantageous adjuvant compounds are the polymers of acrylic or methacrylic acid which are cross-linked, especially with polyalkenyl ethers of sugars or polyalcohols. These compounds are known by the term carbomer (Phameuropa Vol. 8, No. 2, June 1996). Persons skilled in the art can also refer to U.S. Pat. No. 2,909,462 which describes such acrylic polymers cross-linked with a polyhydroxylated compound having at least 3 hydroxyl groups, preferably not more than 8, the hydrogen atoms of at least three hydroxyls being replaced by unsaturated aliphatic radicals having at least 2 carbon atoms. The preferred radicals are those containing from 2 to 4 carbon atoms, e.g. vinyls, allyls and other ethylenically unsaturated groups. The unsaturated radicals may themselves contain other substituents, such as methyl. The products sold under the name Carbopol; (BF Goodrich, Ohio, USA) are particularly appropriate. They are cross-linked with an allyl sucrose or with allyl pentaerythritol. Among then, there may be mentioned Carbopol 974P, 934P and 971P. Most preferred is the use of Cabopol 971P. Among the copolymers of maleic anhydride and alkenyl derivative, are the copolymers EMA (Monsanto), which are copolymers of maleic anhydride and ethylene. The dissolution of these polymers in water leads to an acid solution that will be neutralized, preferably to physiological pH, in order to give the adjuvant solution into which the immunogenic, immunological or vaccine composition itself will be incorporated.

Further suitable adjuvants include, but are not limited to, the RIBI adjuvant system (Ribi Inc.), Block co-polymer (CytRx, Atlanta GA), SAF-M (Chiron, Emeryville CA), monophosphoryl lipid A, Avridine lipid-amine adjuvant, heat-labile enterotoxin from E. coli (recombinant or otherwise), cholera toxin, IMS 1314 or muramyl dipeptide, or naturally occurring or recombinant cytokines or analogs thereof or stimulants of endogenous cytokine release, among many others.

In one aspect, the immunogenic composition is formulated into a water-in-oil emulsion with a suitable adjuvant. The adjuvant can comprise oils and surfactants. In one aspect, the adjuvant is MONTANIDE™ ISA 71R VG (Manufactured by Seppic Inc, Cat no: 365187). In one aspect, the adjuvant is Seppic ISA 206. The adjuvant can be added in an amount of about 100 μg to about 10 mg per dose. Even more preferred the adjuvant is added in an amount of about 100 μg to about 10 mg per dose. Even more preferred the adjuvant is added in an amount of about 500 μg to about 5 mg per dose. Even more preferred the adjuvant is added in an amount of about 750 μg to about 2.5 mg per dose. Most preferred the adjuvant is added in an amount of about 1 mg per dose. In one embodiment, the immunogenic composition of the invention comprises about 7 parts of oil phase containing the adjuvant and about 3 parts of aqueous phase containing the E2 protein of the invention per dose.

In one aspect of the present invention, the mutated 6B8 epitope of the CSFV E2 protein resulted from the replacement as disclosed herein, is used as a marker.

The term “marker” as used herein refers to the mutated 6B8 epitope according to the present invention. The mutant 6B8 epitope according to the present invention is different from the 6B8 epitope sequence of a wildtype CSFV E2 protein (6B8 epitope that has not been genetically modified). Thus, the mutated 6B8 epitope according to the present invention allows the differentiation of naturally infected animals having a non-mutated 6B8 epitope from vaccinated animals having a mutated 6B8 epitope according to the present invention by exemplary immuno tests and/or genomic analytical tests.

In one aspect of the invention, the immunogenic composition of the present invention is a marker vaccine or a DIVA (differentiation between infected and vaccinated animals) vaccine.

The term “marker vaccine” or “DIVA (differentiation between infected and vaccinated animals)” refers to a vaccine having a marker as set forth above. Thus, a marker vaccine can be used for differentiating a vaccinated animal from a naturally infected animal. The immunogenic composition of the present invention acts as a marker vaccine because, in contrast to infection with wild-type CSFV, in animals vaccinated with the vaccine of the present invention the mutated 6B8 epitope according to the present invention can be detected. By exemplary immuno tests and/or genomic analytical tests the mutated 6B8 epitope according to the present invention can be differentiated from the 6B8 epitope sequence of a wildtype CSFV (a 6B8 epitope that has not been genetically modified). Finally, the marker epitope should be specific for the pathogen in order to avoid false-positive serological results which are induced by other organisms that may appear in livestock. However, as the 6B8 epitope is evolutionarily conserved (by sequence alignment) and specific for CSFV (6B8 mAb does not bind to BVDV). Thus, the mutated 6B8 epitope according to the present invention is highly suitable to be used in a marker vaccine.

A major advantage of an efficacious marker vaccine is that it allows the detection of pigs acutely infected or infected some time (for example at least ca. 3 weeks) before taking samples in a vaccinated pig population, and thus offers the possibility to monitor the spread or re-introduction of CSFV in a pig population. Thus, it makes it possible to declare, with a certain level of confidence, that a vaccinated pig population is free of CSFV on the basis of laboratory test results.

The marker vaccine of the present invention is ideally suited for an emergency vaccination in the case of swine fever detection or outbreak. The marker vaccine facilitates fast and effective administration and allows discrimination between animals infected with the field virus (disease-associated) and vaccinated animals.

In one aspect of the present invention, the animals treated with the immunogenic composition of the present invention can be differentiated from animals infected with naturally occurring swine fever virus via analysis of samples obtained from said animals using immuno tests and/or genomic analytical tests.

The term “sample” refers to a sample of a body fluid, to a sample of separated cells or to a sample from a tissue or an organ. Samples of body fluids can be obtained by well-known techniques and include, preferably, samples of blood, plasma, serum, or urine, more preferably, samples of blood, plasma or serum. Tissue or organ samples may be obtained from any tissue or organ by, e.g., biopsy. Separated cells may be obtained from the body fluids or the tissues or organs by separating techniques such as centrifugation or cell sorting.

The term “obtained” may comprise an isolation and/or purification step known to the person skilled in the art, preferably using precipitation, columns etc.

The term “immuno tests” and “genomic analytical tests” are specified below. However, the analysis of said “immuno tests” and “genomic analytical tests”, respectively, is the basis for differentiating animals vaccinated with the immunogenic composition according to the present invention and animals infected with the naturally occurring (disease-associated) swine fever virus.

In one aspect of the present invention said immunogenic composition is formulated for a single-dose administration.

Advantageously, the experimental data provided by the present invention disclose that a single dose administration of the immunogenic composition of the present invention reliably and effectively stimulated a protective immune response. Thus, in one aspect of the invention said immunogenic composition is formulated for and effective by a single-dose administration.

Also, the invention provides the use of the immunogenic composition of the present invention for use as a medicament.

In one aspect, the invention provides a method of preventing and/or treating diseases associated with CSFV in an animal, the method comprising the step of administering the immunogenic composition according to the invention to an animal in need thereof. In one aspect, the disease associated with CSFV is CSF.

The present invention also relates to a method for immunizing an animal, comprising administering to such animal any of the immunogenic compositions according to the present invention. The present invention also relates to a method for immunizing an animal, comprising a single administering to such animal any of the immunogenic compositions according to the present invention. Preferably, the method for immunizing an animal is effective by the single administration of the immunogenic compositions according to the present invention to such animal

The term “immunizing” relates to an active immunization by the administration of an immunogenic composition to an animal to be immunized, thereby causing an immunological response against the antigen included in such immunogenic composition.

The immunization results in lessening of the incidence of the particular CSFV infection in a herd or in the reduction in the severity of clinical signs caused by or associated with the particular CSFV infection. Preferably, the immunization results in lessening of the incidence of the particular CSFV infection in a herd or in the reduction in the severity of clinical signs caused by or associated with the particular CSFV infection by a single administration of the immunogenic composition according to the present invention.

According to one aspect of the invention, the immunization of an animal in need with the immunogenic compositions as provided herewith, results in preventing infection of a subject by CSFV infection, preferably by a single administration of the immunogenic composition according to the present invention. Even more preferably, immunization results in an effective, long-lasting, immunological-response against CSFV infection. It will be understood that the said period of time will last more than 2 months, preferably more than 3 months, more preferably more than 4 months, more preferably more than 5 months, more preferably more than 6 months. It is to be understood that immunization may not be effective in all animals immunized. However, the term requires that a significant portion of animals of a herd are effectively immunized.

Preferably, a herd of animals is envisaged in this context which normally, i.e. without immunization, would develop clinical signs normally caused by or associated with a CSFV infection. Whether the animals of a herd are effectively immunized can be determined without further ado by the person skilled in the art. Preferably, the immunization shall be effective if clinical signs in at least 33%, at least 50%, at least 60%, at least 70%, at least 80%, or at least 90% of the animals of a given herd are lessened in incidence or severity by at least 10%, more preferably by at least 20%, still more preferably by at least 30%, even more preferably by at least 40%, still more preferably by at least 50%, even more preferably by at least 60%, still more preferably by at least 70%, even more preferably by at least 80%, still more preferably by at least 90%, and most preferably by at least 95% in comparison to animals that are either not immunized or immunized with an immunogenic composition that was available prior to the present invention but subsequently infected by CSFV.

In one aspect of the present invention, the animal is swine. In one aspect the animal is a piglet. Piglets are normally younger than 3 to 4 weeks of age. In one aspect the piglets are vaccinated between 1 to 4 weeks of age. In one aspect the animal is a sow. In one aspect the animal is a pregnant sow.

In one aspect of the present invention, the immunogenic composition is administered intradermal, intratracheal, intravaginal, intramuscular, intranasal, intravenous, intraarterial, intraperitoneal, oral, intrathecal, subcutaneous, intracutaneous, intracardial, intralobal, intramedullar, intrapulmonary, and combinations thereof. However, depending on the nature and mode of action of a compound, the immunogenic composition may be administered by other routes as well.

The present invention also provides a method of reducing the incidence of or severity in an animal of one or more clinical signs associated with CSF, the method comprising the step of administering the immunogenic composition according to the present invention to an animal in need thereof, wherein the reduction of the incidence of or the severity of the one or more clinical signs is relative to an animal not receiving the immunogenic composition. Preferably, the method comprises the administration of a single dose of the immunogenic composition and is effective in reduction of the incidence of or the severity of the one or more clinical signs by such single administration of the immunogenic composition.

The term “clinical signs” as used herein refers to signs of infection of an animal from CSFV. The clinical signs are defined further below. However, the clinical signs also include but are not limited to clinical signs that are directly observable from a live animal. Examples for clinical signs that are directly observable from a live animal include nasal and ocular discharge, lethargy, coughing, wheezing, thumping, elevated fever, weight gain or loss, dehydration, diarrhea, joint swelling, lameness, wasting, paleness of the skin, unthriftiness, and the like. Mittelholzer et al. (Vet. Microbiol., 2000. 74(4): p. 293-308) developed a checklist for the determination of the clinical scores in CSF animal experiments. This checklist contains the parameters liveliness, body tension, body shape, breathing, walking, skin, eyes/conjunctiva, appetite, defecation and leftovers in feeding through.

Preferably, clinical signs are lessened in incidence or severity by at least 10%, more preferably by at least 20%, still more preferably by at least 30%, even more preferably by at least 40%, still more preferably by at least 50%, even more preferably by at least 60%, still more preferably by at least 70%, even more preferably by at least 80%, still more preferably by at least 90%, and most preferably by at least 95% in comparison to subjects that are either not treated or treated with an immunogenic composition that was available prior to the present invention but subsequently infected by CSFV.

In one aspect of the invention the immunogenic composition is administered once and is efficacious by such single administration.

However, while the single dose administration is preferred, the immunogenic composition can also be administered twice or several times, with a first dose being administered prior to the administration of a second (booster) dose. Preferably, the second dose is administered at least 15 days after the first dose. More preferably, the second dose is administered between 15 and 40 days after the first dose. Even more preferably, the second dose is administered at least 17 days after the first dose. Still more preferably, the second dose is administered between 17 and 30 days after the first dose. Even more preferably, the second dose is administered at least 19 days after the first dose. Still more preferably, the second dose is administered between 19 and 25 days after the first dose. Most preferably the second dose is administered at least 21 days after the first dose. In a preferred aspect of the two-time administration regimen, both the first and second doses of the immunogenic composition are administered in the same amount. In addition to the first and second dose regimen, an alternate embodiment comprises further subsequent doses. For example, a third, fourth, or fifth dose could be administered in these aspects. Preferably, subsequent third, fourth, and fifth dose regimens are administered in the same amount as the first dose, with the time frame between the doses being consistent with the timing between the first and second doses mentioned above.

In one aspect of the invention the one or more clinical signs are selected from the group consisting of: respiratory distress, labored breathing, coughing, sneezing, rhinitis, tachypnea, dyspnea, pneumonia, red/blue discolouration of the ears and vulva, jaundice, lymphocytic infiltrates, lymphadenopathy, hepatitis, nephritis, anorexia, fever, lethargy, agalatia, diarrhea, nasal extrudate, conjunctivitis, progressive weight loss, reduced weight gain, paleness of the skin, gastric ulcers, macroscopic and microscopic lesions on organs and tissues, lymphoid lesions, mortality, virus induced abortion, stillbirth, malformation of piglets, mummification and combinations thereof.

In one aspect, the present invention also provides a method of differentiating animals infected with CSFV from animals vaccinated with the immunogenic composition according to the present invention, comprising
a) obtaining a sample, and
b) testing said sample in an immuno test.

The term “immuno test” refers to a test comprising an antibody specific for the 6B8 epitope of the E2 protein of the CSFV. The antibody may be specific for the mutated 6B8 epitope according to the present invention or for the 6B8 epitope of a wildtype CSFV E2 protein (6B8 epitope that has not been genetically modified). However, the term “immuno test” does also refer to a test comprising mutated 6B8 epitope peptides according to the present invention or 6B8 epitope peptides of a wildtype CSFV E2 protein (6B8 epitope that has not been genetically modified). Examples of immuno tests include any enzyme-immunological or immunochemical detection method such as ELISA (enzyme linked immunosorbent assay), double competitive ELISA, EIA (enzyme immunoassay), RIA (radioimmunoassay), sandwich enzyme immune tests, fluorescent antibody test (FAT), electrochemiluminescence sandwich immunoassays (ECLIA), dissociation-enhanced lanthanide fluoro immuno assay (DELFIA) phase immune tests, immunofluorescent test (IFT), immunohistological staining, Western blot analysis or any other suitable method available to technicians skilled in the art. Depending upon the assay used, the antigens or the antibodies can be labeled by an enzyme, a fluorophore or a radioisotope. See, e.g., Coligan et al. Current Protocols in Immunology, John Wiley & Sons Inc., New York, N.Y. (1994); and Frye et al., Oncogen 4:1153-1157, 1987.

Preferably, an antibody specific for the 6B8 epitope of a wildtype CSFV E2 protein is used to detect CSFV antigen in serum cells (such as leucocytes) or cryostat sections of isolated organs (such as tonsils, spleen, kidney, lymph nodes, distal portions of the ileum) from an animal (such as a pig) that is suspected to be infected with wildtype CSFV or that is vaccinated with a vaccine comprising a recombinant CSFV E2 protein according to the invention. In such a case, only the sample of the animal infected with wildtype CSFV will show positive results by said 6B8 epitope specific antibody. In contrast, the sample of an animal vaccinated with the vaccine comprising a recombinant CSFV E2 protein of the present invention will show no results by said 6B8 epitope specific antibody due to the mutation within the 6B8 epitope according to the present invention. In an alternative test, CSFV is isolated from, for example, organs (such as the tonsils of an animal) or serum cells (such as leukoyctes) infected, suspected to be infected with wildtype CSFV or vaccinated animals and incubated with a suitable cell line (such as SK-6 cells or PK-15 cells) for infection of the cells with the virus. The replicated virus is subsequently detected in the cells using 6B8 epitope specific antibodies that differentiate between the field (wildtype, disease associated) CSFV and the recombinant CSFV according to the invention. Further, peptides could be used to block unspecific cross-reactivity. Moreover, antibodies specific for other epitopes of the wildtype CSFV could be used as a positive control.

More preferably, an ELISA is used, wherein the antibody specific for the 6B8 epitope of a wildtype CSFV E2 protein (6B8 epitope that has not been genetically modified) is cross-linked to micro-well assay plates for differentiating between infected pigs from pigs vaccinated with the vaccine according to the present invention. Said cross-linking preferably is performed through an anchor protein such as, for example, poly-L-lysine. ELISAs employing such cross-linking are in general more sensitive when compared to ELISAs employing a passively coated technique. The wildtype (disease associated) CSFV binds to the antibody specific for the 6B8 epitope of a wildtype CSFV E2 protein (6B8 epitope that has not been genetically modified). The detection of the binding of the wildtype CSFV virus to the antibody specific for the 6B8 epitope of a wildtype CSFV can be performed by a further antibody specific for CSFV. In such a case, only the sample of the infected pig will show positive results by the 6B8 epitope specific antibody. Further, peptides could be used to block unspecific cross-reactivity. Moreover, antibodies specific for other epitopes of the wildtype CSFV could be used as a positive control.

Alternatively, the micro-well assay plates may be cross-linked with an antibody specific for CSFV other than the antibody specific for the 6B8 epitope of a wildtype CSFV E2 protein (6B8 epitope that has not been genetically modified). The wildtype (disease associated) CSFV binds to the cross linked antibody. The detection of the binding of the wildtype CSFV to the cross linked antibody can be performed by the antibody specific for the 6B8 epitope of a wildtype CSFV E2 protein (6B8 epitope that has not been genetically modified).

As already set forth above the 6B8 epitope is evolutionarily conserved and specific for wildtype CSFV.

Therefore, more preferably, an ELISA is used for detecting in the sample antibodies that are directed against the mutant 6B8 epitope according to the present invention or the 6B8 epitope of a wildtype CSFV (6B8 epitope that has not been genetically modified). Such a test comprises mutant 6B8 epitope peptides according to the present invention or the 6B8 epitope peptides of a wildtype CSFV (6B8 epitope that has not been genetically modified).

Such a test could e.g. comprise wells with a mutant 6B8 epitope according to the present invention or the 6B8 epitope of a wildtype CSFV (6B8 epitope that has not been genetically modified) cross-linked to micro-well assay plates. Said cross-linking preferably is performed through an anchor protein such as, for example, poly-L-lysine. Expression systems for obtaining a mutant or wildtype 6B8 epitope are well known to the person skilled in the art. Alternatively, said 6B8 epitopes could be chemically synthesized. It has to be understood that although the mutant or wildtype 6B8 epitope as such can be used in a test according to the invention, it can be convenient to use a protein comprising the complete E2 protein or a fragment of the E2 protein comprising the said 6B8 epitope, instead of the relatively short epitope as such. Especially when the epitope is for example used for the coating of a well in a standard ELISA test, it may be more efficient to use a larger protein comprising the epitope, for the coating step.

Animals vaccinated with the vaccine comprising a recombinant CSFV E2 protein according to the present invention have not raised antibodies against the wild-type 6B8 epitope. However, such animals have raised antibodies against the mutant 6B8 epitope according to the present invention. As a consequence, no antibodies bind to a well coated with the wildtype 6B8 epitope. In contrast, if a well has been coated with the mutant 6B8 epitope according to the present invention antibodies bind to said mutant 6B8 epitope.

Animals infected with the wild-type CSFV will however have raised antibodies against the wild-type epitope of CSFV. However, such animals have not raised antibodies against the mutant 6B8 epitope according to the present invention. As a consequence, no antibodies bind to a well coated with the mutant 6B8 epitope according to the present invention. In contrast, if a well has been coated with the wildtype 6B8 epitope antibodies bind to the wildtype 6B8 epitope.

The binding of the antibodies to the mutant 6B8 epitope according to the present invention or the 6B8 epitope of a wildtype CSFV (6B8 epitope that has not been genetically modified) can be done by methods well known to the person skilled in the art.

Preferably, the ELISA is a sandwich type ELISA. More preferably, the ELISA is a competitive ELISA. Most preferably, the ELISA is a double competitive ELISA. However, the different ELISA techniques are well known to the person skilled in the art. ELISA have been described exemplary by Wensvoort G. et al., 1988 (Vet. Microbiol. 17(2): 129-140), by Robiolo B. et al., 2010 (J. Virol. Methods. 166(1-2): 21-27) and by Colijn, E. O. et al., 1997 (Vet. Microbiology 59:15-25).

In one aspect of the present invention the immuno test comprises testing whether antibodies specifically recognizing the intact 6B8 epitope of the CSFV E2 protein are binding to the CSFV E2 protein in the sample. In one aspect of the present invention the immuno test comprises testing whether an antibody specifically recognizing a 6B8 epitope of the CSFV E2 protein is present in the sample, and/or testing whether an antibody specifically recognizing a mutated 6B8 epitope of the CSFV E2 protein is present in the sample. Such a mutated 6B8 epitope comprises mutation(s) in the 6B8 epitope as defined herein.

In one aspect of the present invention the immunological test is an EIA (enzyme immunoassay) or ELISA (enzyme linked immunosorbent assay). In one aspect of the present invention the ELISA is an indirect ELISA, Sandwich ELISA, a competitive ELISA or double competitive ELISA, preferably a double competitive ELISA.

In one aspect, the present invention also provides a nucleic acid coding for the recombinant CSFV E2 protein according to the present invention.

The term “nucleic acid” refers to polynucleotides including DNA molecules, RNA molecules, cDNA molecules or derivatives. The term encompasses single as well as double stranded polynucleotides. The nucleic acid of the present invention encompasses recombinant polynucleotides (i.e. recombinant from its natural context) and genetically modified forms. Moreover, comprised are also chemically modified polynucleotides including naturally occurring modified polynucleotides such as glycosylated or methylated polynucleotides or artificial modified one such as biotinylated polynucleotides. Further, it is to be understood that the recombinant CSFV E2 protein of the present invention may be encoded by a large number of polynucleotides due to the degenerated genetic code. In one aspect of the present invention, the nucleic acid coding for the recombinant CSFV E2 protein according to the present invention is codon-optimized according to the host cell into which the nucleic acid is to be introduced.

In one aspect, the present invention also provides a vector comprising the nucleic acid coding for the recombinant CSFV E2 protein according to the present invention. In one aspect, the vector is an expression vector.

The term “vector” encompasses phage, plasmid, viral or retroviral vectors as well artificial chromosomes, such as bacterial or yeast artificial chromosomes. Moreover, the term also relates to targeting constructs which allow for random or site-directed integration of the targeting construct into genomic DNA. Such target constructs, preferably, comprise DNA of sufficient length for either homologous or heterologous recombination as described in detail below. The vector encompassing the nucleic acid of the present invention, preferably, further comprises selectable markers for propagation and/or selection in a host. The vector may be incorporated into a host cell by various techniques well known in the art. For example, a plasmid vector can be introduced in a precipitate such as a calcium phosphate precipitate or rubidium chloride precipitate, or in a complex with a charged lipid or in carbon-based clusters, such as fullerenes. Alternatively, a plasmid vector may be introduced by heat shock or electroporation techniques. Should the vector be a virus, it may be packaged in vitro using an appropriate packaging cell line prior to application to host cells. Retroviral vectors may be replication competent or replication defective. In the latter case, viral propagation generally will occur only in complementing host/cells. More preferably, the polynucleotide is operatively linked to expression control sequences allowing expression in prokaryotic or eukaryotic cells or isolated fractions thereof. Expression of said polynucleotide comprises transcription of the polynucleotide, preferably into a translatable mRNA. Regulatory elements ensuring expression in eukaryotic cells, preferably mammalian cells, are well known in the art. They, preferably, comprise regulatory sequences ensuring initiation of transcription and, optionally, poly-A signals ensuring termination of transcription and stabilization of the transcript. Additional regulatory elements may include transcriptional as well as translational enhancers. Possible regulatory elements permitting expression in prokaryotic host cells comprise, e.g., the lac, trp or tac promoter in E. coli, and examples for regulatory elements permitting expression in eukaryotic host cells are the AOX1 or GAL1 promoter in yeast or the CMV-, SV40-, RSV-promoter (Rous sarcoma virus), CMV-enhancer, SV40-enhancer or a globin intron in mammalian and other animal cells. Moreover, inducible expression control sequences may be used in an expression vector encompassed by the present invention. Such inducible vectors may comprise tet or lac operator sequences or sequences inducible by heat shock or other environmental factors. Suitable expression control sequences are well known in the art. For example, the techniques are described in Sambrook, Molecular Cloning A Laboratory Manual, Cold Spring Harbor Laboratory (1989) N.Y. and Ausubel, Current Protocols in Molecular Biology, Green Publishing Associates and Wiley Interscience, N.Y. (1994). Preferably, the vector of the invention is a baculovirus vector. More preferably, the vector of the invention is a mammalian expression vector for the expression in a mammalian cell, such as a CHO cell.

In one aspect, the invention also provides a host cell comprising the nucleic acid or vector of the invention. The host cell may be a prokaryotic cell, such as E. coli, or an eukaryotic cell, such as for example an inset cell. Preferably, the host cell is an SF9 cell. More preferably, the host cell is a mammalian cell, such as a CHO cell.

In one aspect, the invention also provides a method for producing a recombinant CSFV E2 protein of the invention, comprising
(i) culturing the host cell, preferably the CHO cell as defined herein under conditions suitable for the expression of the CSFV E2 protein, and
(ii) isolating and optionally purifying the CSFV E2 protein.

The purification of the CSFV E2 protein, for example can be done via a fusion tag or fusion peptide attached to CSFV E2 protein, e.g. via a His-tag, FLAG-tag or a Fc-fragment, if present.

In one aspect, the invention also provides a method of preparing an immunogenic composition, comprising: (i) culturing cells containing an expression vector capable of expressing an E2 protein; and (ii) harvesting the E2 protein or the whole cell culture comprising the E2 protein, wherein the E2 protein is defined herein above.

In one aspect of the invention, the expression vector is a recombinant mammalian expression vector comprising the nucleic acid molecule of the invention. In one aspect, the recombinant mammalian expression vector is derived from a commercial product. In one aspect, the recombinant mammalian expression vector is derived from pcDNA3.4 (Invitrogen). In one aspect, the cells are mammalian cells. In one aspect, the mammalian cells are CHO cells.

In one aspect, the method is performed by using the ExpiCHO™ Expression System (Gibico, Cat #A29133) according to the user manual (Revision D.0, May 25, 2018, Thermo Fisher Scientific Inc). In one embodiment, the CHO cells are ExpiCHO-S™ cells sold under Cat #A29127 by Gibico. In one embodiment, the method comprises a step of infecting the mammalian cells with the recombinant mammalian expression vector of the invention, for example, using ExpiFectamine™ CHO Transfection Kit (Gibico, Cat #A29129).

In one aspect of the invention, the expression vector is a recombinant baculovirus comprising the nucleic acid molecule of the invention. In one aspect, the recombinant baculovirus is derived from a commercial product. In one aspect, the recombinant baculovirus is derived from a commercial product sold under the trademark Sapphire™ Baculovirus (Allele Biotechnology). In one aspect, the cells are insect cells. In one aspect, the insect cells are SF+ cells. In one embodiment, the SF+ cells are a commercial product sold by Protein Sciences Corporation (Meriden, CT).

In one aspect of the invention, the method comprises a step of preparing a recombinant baculovirus comprising the nucleic acid molecule of the invention. In one aspect, the recombinant baculovirus is derived from a commercial product. In one aspect, the recombinant baculovirus is derived from a commercial product sold under the trademark Sapphire™ Baculovirus (Allele Biotechnology).

In one aspect of the invention, the method comprises a step of infecting cells with the recombinant baculovirus of the invention. In one embodiment, the cells are insect cells. In one embodiment, the insect cells are SF+ cells. In one embodiment, the SF+ cells are a commercial product sold by Protein Sciences Corporation (Meriden, CT).

In one aspect of the invention, the method comprises preparing a recombinant baculovirus comprising the nucleic acid molecule of the invention, and infecting insect cells with the recombinant baculovirus. In one embodiment, the recombinant baculovirus is derived from a commercial product sold under the trademark Sapphire™ Baculovirus (Allele Biotechnology). In one embodiment, the insect cells are SF+ cells. In one embodiment, the SF+ cells are a commercial product sold by Protein Sciences Corporation (Meriden, CT).

In one aspect of the invention, the method comprises: (i) preparing a recombinant baculovirus comprising the nucleic acid molecule of the invention; (ii) infecting insect cells with the recombinant baculovirus; (iii) culturing the insect cells in a culture medium; and (iv) harvesting the E2 protein of the invention or the whole cell culture comprising the E2 protein of the invention. In one aspect, the recombinant baculovirus is derived from a commercial product sold under the trademark Sapphire™ Baculovirus (Allele Biotechnology). In one embodiment, the insect cells are SF+ cells. In one embodiment, the SF+ cells are a commercial product sold by Protein Sciences Corporation (Meriden, CT).

In one aspect of the invention, the culture medium for culturing the cells of the invention will be determined by those of skill in the art. In one aspect, the culture medium is a serum-free insect cell medium. In one aspect, the culture medium is Ex-CELL 420 (Ex-CELL® 420 serum-free medium for insect cells, Sigma-Aldrich, Cat. 14420C).

In one aspect of the invention, the insect cells are cultured under the condition suitable for the expression of the E2 protein. In one aspect, the insect cells are incubated over a period of up to ten days, preferably from about two days to about ten days, more preferably from about four days to about nine days, and even more preferably from about five days to about eight days. In one aspect, the condition suitable for culturing the insect cell comprises a temperature between about 22-32° C., preferably from about 24-30° C., more preferably from about 25-29° C., even more preferably from about 26-28° C., and most preferably about 27° C.

In one aspect of the invention, the method further comprises a step of inactivating the cell culture of the invention. Any conventional inactivation method can be used for purposes of the invention, including but not limited to chemical and/or physical treatments.

In one aspect, the inactivation step comprises the addition of cyclized binary ethylenimine (BEI), preferably in a concentration of about 1 to about 20 mM, preferably of about 2 to about 10 mM, more preferably of about 5 mM or 10 mM. In one embodiment, the inactivation step comprises the addition of a solution of 2-bromoethyleneamine hydrobromide which will be cyclized to form BEI in NaOH.

In one aspect, the inactivation step is performed between 25-40° C., preferably between 28-39° C., more preferably between 30-39° C., more preferably between 35-39° C. In one embodiment, inactivation step is performed for 24-72 h, preferably for 30-72 h, more preferably 48-72 h. In general, the inactivation step is performed until no replication of the viral vector is detectable.

In one aspect of the invention, the method further comprises a step of a neutralization step after the inactivation step. The neutralization step comprises adding of an equivalent amount of an agent that neutralizes the inactivation agent within the solution. In one embodiment, the inactivation agent is BEI. In one aspect, the neutralization agent is sodium thiosulfate. In one aspect, when the inactivation agent is BEI, an equivalent amount of sodium thiosulfate will be added. For example, in the event BEI is added to a final concentration of 5 mM, a 1.0M sodium thiosulfate solution is added to give a final minimum concentration of 5 mM to neutralize any residual BEI. In one aspect, the neutralization step comprises adding of a sodium thiosulfate solution to a final concentration of 1 to 20 mM, preferably of 2 to 10 mM, more preferably of 5 mM or 10 mM, when the inactivation agent is BEI. In one aspect, the neutralization agent is added after the inactivation step is completed, which means that no replication of the viral vector replication can be detected. In one aspect, the neutralization agent is added after the inactivation step is performed for 24 h. In one aspect, the neutralization agent is added after the inactivation step is performed for 30 h. In one aspect, the neutralization agent is added after the inactivation step is performed for 48 h. In one aspect, the neutralization agent is added after the inactivation step is performed for 72 h.

In one aspect of the invention, the CSFV E2 protein is further purified. For example, the CSFV E2 protein according to the invention may comprise a fusion peptide such as for example a His-tag, a FLAG-tag or an Fc-fragment, which are attached to the CSFV E2 protein. CSFV E2 proteins with a His-tag can for example be purified by Ni-NTA affinity (nickel²⁺-ion coupled to nitrilotriacetic acid). CSFV E2 proteins with a FLAG-tag (such as DYKDDDDK) can for example be purified by a monoclonal antibody specifically binds to the FLAG-tag (commercial kits for example are available from Sigma-Aldrich (Anti-FLAG® M1 Agarsose affinity gel)). CSFV E2 proteins linked to an Fc-fragment can for example be purified either by protein A or protein G affinity.

In one aspect, the invention also provides a method for producing a recombinant CSFV E2 protein of the invention, in CHO cells, said method comprises
a) growing CHO cells in a medium;
b) transfecting cells from step a) with a recombinant mammalian expression vector comprising a nucleic acid molecule encoding the recombinant CSFV E2 protein of the invention,
c) culturing the transfected CHO cells from step b) under conditions suitable for the expression of the recombinant CSFV E2 protein;
d) harvesting and optionally purifying the recombinant CSFV E2 protein.

In one embodiment, the CHO cells are adapted to grow to high-density suspension culture in a medium.

In one embodiment, the recombinant mammalian expression vector is derived from pcDNA3.4 (Invitrogen).

In one embodiment, the CHO cells are ExpiCHO-S™ cells sold under Cat #A29127 by Gibico.

In one embodiment, the medium as used in the method is the ExpiCHO™ Expression Medium sold under Cat #A29100 by Gibico.

In one embodiment, the adapted CHO cells are transfected by using ExpiFectamine™ CHO Transfection Kit (Gibico, Cat #A29129).

In one embodiment, in step c), the transfected CHO cells are cultured at about 32-37° C., preferably about 32° C.

In one embodiment, in step c), the transfected CHO cells are cultured with a humidified atmosphere of about 5-8% CO₂.

In one embodiment, ExpiFectamine™ CHO Enhancer and ExpiCHO™ Feed are added in step c).

In one embodiment, the recombinant CSFV E2 protein is harvested about 2-14 days post transfection, for example, about 4-12 days post transfection, or about 8-10 days post transfection.

In one aspect, the present invention provides a kit for differentiating animals infected with CSFV from animals vaccinated with the immunogenic composition of the invention. In one aspect, the kit comprises the antibody as defined herein or an antigen-binding fragment thereof, the recombinant E2 protein of the invention with mutated 6B8 epitope, and/or a wild type E2 protein of CSFV comprising the 6B8 epitope as defined herein. The kit may also contain instructions for use.

In one aspect of the invention, the CSFV E2 protein is further purified. For example, the CSFV E2 protein according to the invention may comprise a fusion peptide such as for example a His-tag, a FLAG-tag or an Fc-fragment, which are attached to the CSFV E2 protein. CSFV E2 proteins with a His-tag can for example be purified by Ni-NTA affinity (nickel²⁺-ion coupled to nitrilotriacetic acid). CSFV E2 proteins with a FLAG-tag (such as DYKDDDDK) can for example be purified by a monoclonal antibody specifically binds to the FLAG-tag (commercial kits for example are available from Sigma-Aldrich (Anti-FLAG® M1 Agarsose affinity gel)). CSFV E2 proteins linked to a Fc-fragment can for example be purified either by protein A or protein G affinity.

The following clauses are also described herein and part of disclosure of the invention:

### CLAUSES

The following clauses are also described herein and part of disclosure of the invention:
1. A recombinant CSFV (classical swine fever virus) E2 protein in which a fragment comprising at least one of the amino acids defining the 6B8 epitope of the CSFV E2 protein is replaced by a corresponding fragment of the E2 protein from a pestivirus other than CSFV.
2. The recombinant CSFV E2 protein according to clause 1, wherein the replacement results in a mutated 6B8 epitope in the recombinant CSFV E2 protein to which the binding of the 6B8 monoclonal antibody or the antigen-binding fragment thereof is specifically inhibited.
3. The recombinant CSFV E2 protein according to clauses 1 or 2, wherein the recombinant CSFV E2 protein is derived from a wildtype CSFV E2 protein from a strain selected from C-strain, QZ07, GD18 and GD191.
4. The recombinant CSFV E2 protein according to any one of clause 1-3, wherein the wildtype CSFV E2 protein comprises an amino acid sequence selected from SEQ ID NOs: 9-12.
5. The recombinant CSFV E2 protein according to any one of clauses 1-4, wherein the 6B8 epitope of the CSFV E2 protein is specifically recognized and/or bound by the 6B8 monoclonal antibody which (i) is produced by a hybridoma deposited at CCTCC under the accession number CCTCC C2018120, or
(ii) comprises a heavy chain variable region (VH) having an amino acid sequence as set forth in SEQ ID NO: 7 and a light chain variable region (VL) having an amino acid sequence as set forth in SEQ ID NO: 8, or
(iii) comprises the CDRs of the monoclonal antibody produced by a hybridoma deposited at CCTCC under the accession number CCTCC C2018120, or
(iv) comprises a VH CDR1 comprising the amino acid sequence set forth in SEQ ID NO:1, a VH CDR2 comprising the amino acid sequence set forth in SEQ ID NO:2, a VH CDR3 comprising the amino acid sequence set forth in SEQ ID NO:3, a VL CDR1 comprising the amino acid sequence set forth in SEQ ID NO:4, a VL CDR2 comprising the amino acid sequence set forth in SEQ ID NO:5, and a VL CDR3 comprising the amino acid sequence set forth in SEQ ID NO:6.
6. The recombinant CSFV E2 protein according to any one of clauses 1-5, wherein the amino acids defining the 6B8 epitope of the CSFV E2 protein at least comprises the amino acid at position 14, position 22, position 24, positions 24/25, position 10, position 41 and/or position 64 of the CSFV E2 protein.
7. The recombinant CSFV E2 protein according to any one of clauses 1-6, wherein the fragment comprising at least one of the amino acids defining the 6B8 epitope of the CSFV E2 protein is the B/C domain or a fragment thereof of the CSFV E2 protein.
8. The recombinant CSFV E2 protein according to any one of clauses 1-7, wherein
i) the sequence of amino acid position 11 to amino acid position 38 of the CSFV E2 protein;
ii) the sequence of amino acid position 11 to amino acid position 39 of the CSFV E2 protein;
iii) the sequence of amino acid position 11 to amino acid position 56 of the CSFV E2 protein;
iv) the sequence of amino acid position 11 to amino acid position 80 of the CSFV E2 protein;
v) the sequence of amino acid position 11 to amino acid position 90 of the CSFV E2 protein;
vi) the sequence of amino acid position 11 to amino acid position 109 of the CSFV E2 protein; or
vii) the sequence of amino acid position 11 to amino acid position 110 of the CSFV E2 protein
is replaced by a corresponding sequence of the E2 protein from a pestivirus other than CSFV.
9. The recombinant CSFV E2 protein according to any one of clauses 1-8, wherein the pestivirus other than CSFV is selected from bovine viral diarrahea viruses comprising BVDV-2; border disease viruses comprising Switzerland, BDV-1, BDV-2, BDV-3, BDV-4, BDV-5, BDV-6, Chamois, Italy, Turkey, and Tunisian sheep virus (TSV); and atypical pestiviruses comprising Giraffe pestivirus, BVDV-3, Pronghorn antelope, Bungowannah virus and Norway rat pestivirus.
10. The recombinant CSFV E2 protein according to clause 9, wherein the pestivirus other than CSFV is selected from Giraffe pestivirus PG2 strain, BDV-2 Reindeer V60, Norway rat pestivirus isolate NrPV/NYC-D23, TSV9552, Bungowannah 6778, and BVDV3 Hobi-like Th/04 KhonKaen.
11. The recombinant CSFV E2 protein according to any one of clauses 1-10, wherein the E2 protein from a pestivirus other than CSFV comprises the amino acid sequence selected from SEQ ID NOs: 21-26.
12. The recombinant CSFV E2 protein according to any one of clauses 1-11, which comprises at least one amino acid residue at amino acid positions defining the 6B8 epitope of the CSFV E2 protein which specifically inhibit the binding of 6B8 monoclonal antibody to the CSFV E2 protein.
13. The recombinant CSFV E2 protein according to clause 12,
   i. wherein the recombinant CSFV E2 protein comprises the amino acid mutations at the amino acid positions as defined in tables 1 to 7,
   ii. wherein the amino acid at position 24 of the recombinant CSFV E2 protein is R, D or I, with R being preferred,
   iii. the amino acid at positions 24 and 25 of the recombinant CSFV E2 protein is R, D or I, with R being preferred, and D, K, L, N, R, T, V, E or P, with D, K, L, N, R, T and V being preferred, respectively,
   iv. the amino acid at position 14 of the recombinant CSFV E2 protein is K, A, E, Q or R, with K being preferred,
   v. the amino acid at position 22 of the recombinant CSFV E2 protein is A, R, Q, E, D, N, K, L, P, T, V or S, with A, Q, D, E, N and S being preferred,
   vi. the amino acid at position 10 of the recombinant CSFV E2 protein is A or P,
   vii. the amino acid at position 41 of the recombinant CSFV E2 protein is A, N or E, and/or
   viii. the amino acid at position 64 of the recombinant CSFV E2 protein is A, S, E, D, G, H, T, L, P, K or W, with A, K, and W being preferred.
14. The recombinant CSFV E2 protein according to any one of clauses 1-13, wherein the amino acid at position 24 of the recombinant CSFV E2 protein is R, D or I, with R being preferred, the amino acid at positions 24 and 25 of the recombinant CSFV E2 protein is R, D or I, with R being preferred, and D, K, L, N, R, T, V, E or P, with D, K, L, N, R, T and V being preferred, respectively, the amino acid at position 14 of the recombinant CSFV E2 protein is K, A, E, Q or R, with K being preferred, and the amino acid at position 22 of the recombinant CSFV E2 protein is A, R, Q, E, D, N, K, L, P, T, V or S, with A, Q, D, E, N and S being preferred.
15. The recombinant CSFV E2 protein according to clause 14, wherein the amino acid at position 24 of the recombinant CSFV E2 protein is R, the amino acid at positions 25 of the recombinant CSFV E2 protein is D, the amino acid at position 14 of the recombinant CSFV E2 protein is K, and the amino acid at position 22 of the recombinant CSFV E2 protein is A.
16. The recombinant CSFV E2 protein according to any one of clauses 1 to 15, wherein the recombinant CSFV E2 protein comprises an amino acid sequence selected from the group consisting of SEQ ID NOs: 27-48 and 71-74.
17. The recombinant CSFV E2 protein according to any one of clauses 1 to 16, wherein an Fc fragment, such as a swine Fc fragment is linked to the recombinant CSFV E2 protein; preferably the Fc fragment, such as a swine Fc fragment is linked to the C terminus of the E2 protein, preferably via a peptide linker.
18. The recombinant CSFV E2 protein according to clause 17, wherein the Fc fragment comprises an amino acid sequence of SEQ ID NO:20.
19. The recombinant CSFV E2 protein according to clause 17 or 18, wherein the peptide linker comprises an amino acid sequence selected from SEQ ID NOs: 16-19.
20. The recombinant CSFV E2 protein according to any one of clauses 17-19, wherein the recombinant CSFV E2 protein comprises an amino acid sequence selected from the group consisting of SEQ ID NOs: 49-70 and 75-78; preferably selected from the group consisting of SEQ ID NOs: 53, 68 and 75; more preferably preferably selected from the group consisting of SEQ ID NOs: 53 and 75.
21. A recombinant CSFV E2 protein, wherein the recombinant CSFV E2 protein comprises an amino acid sequence of the E2 protein of CSFV field strain QZ07 with a fragment comprising at least one of the amino acids defining the 6B8 epitope being replaced by a corresponding fragment of the E2 protein of BVDV3 Hobi-like Th/04 KhonKaen strain.
22. The recombinant CSFV E2 protein according to clause 21,
wherein the sequence of amino acid position 11 to amino acid position 38 of the CSFV E2 protein is replaced by a corresponding sequence of the E2 protein from BVDV3 Hobi-like Th/04 KhonKaen strain, and the numbering of the amino acid position is determined based on the position numbers of the amino acid sequence of the E2 protein of CSFV field strain QZ07 shown by SEQ ID NO: 10; or
wherein the sequence of amino acid position 11 to amino acid position 39 of the CSFV E2 protein is replaced by a corresponding sequence of the E2 protein from BVDV3 Hobi-like Th/04 KhonKaen strain, and the numbering of the amino acid position is determined based on the position numbers of the amino acid sequence of the E2 protein of CSFV field strain QZ07 shown by SEQ ID NO: 10; or
wherein the sequence of amino acid position 11 to amino acid position 56 of the CSFV E2 protein is replaced by a corresponding sequence of the E2 protein from BVDV3 Hobi-like Th/04 KhonKaen strain, and the numbering of the amino acid position is determined based on the position numbers of the amino acid sequence of the E2 protein of CSFV field strain QZ07 shown by SEQ ID NO: 10; or
wherein the sequence of amino acid position 11 to amino acid position 80 of the CSFV E2 protein is replaced by a corresponding sequence of the E2 protein from BVDV3 Hobi-like Th/04 KhonKaen strain, and the numbering of the amino acid position is determined based on the position numbers of the amino acid sequence of the E2 protein of CSFV field strain QZ07 shown by SEQ ID NO: 10; or
wherein the sequence of amino acid position 11 to amino acid position 90 of the CSFV E2 protein is replaced by a corresponding sequence of the E2 protein from BVDV3 Hobi-like Th/04 KhonKaen strain, the numbering of the amino acid position is determined based on the position numbers of the amino acid sequence of the E2 protein of CSFV field strain QZ07 shown by SEQ ID NO: 10; or
wherein the sequence of amino acid position 11 to amino acid position 109 of the CSFV E2 protein is replaced by a corresponding sequence of the E2 protein from BVDV3 Hobi-like Th/04 KhonKaen strain, and the numbering of the amino acid position is determined based on the position numbers of the amino acid sequence of the E2 protein of CSFV field strain QZ07 shown by SEQ ID NO: 10.
23. The recombinant CSFV E2 protein according to clause 21 or 22, wherein compared to the corresponding fragment of the E2 protein of BVDV3 Hobi-like Th/04 KhonKaen strain, the recombinant CSFV E2 protein further comprises at least one amino acid mutation at positions 10, 14, 22, 24, 25, 24/25, 41 and/or 64; preferably, the recombinant CSFV E2 protein further comprises the amino acid mutations at the amino acid positions as defined in tables 1 to 7; preferably wherein the recombinant CSFV E2 protein further comprises one single amino acid mutation at position 14, 24, 25 or 64 of the recombinant CSFV E2 protein.
24. The recombinant CSFV E2 protein according to clause 23, wherein
   the amino acid at position 14 of the recombinant CSFV E2 protein is K, A, E, Q or R, with K being preferred;
   the amino acid at position 64 of the recombinant CSFV E2 protein is A, S, E, D, G, H, T, L, P, K or W, with K being preferred;
   the amino acid at position 24 of the recombinant CSFV E2 protein is R, D or I, with R being preferred;
   the amino acid at position 25 of the recombinant CSFV E2 protein is D, K, L, N, R, T, V, E or P, with D being preferred;
   the amino acid at position 25 of the recombinant CSFV E2 protein is D, K, L, N, R, T, V, E or P, with N being preferred; or
   the amino acid at position 25 of the recombinant CSFV E2 protein is D, K, L, N, R, T, V, E or P, with R being preferred.
25. The recombinant CSFV E2 protein according to any one of clauses 21-24, wherein an Fc fragment, such as a swine Fc fragment, is linked to the recombinant CSFV E2 protein; preferably the Fc fragment, such as a swine Fc fragment, is linked to the C terminus of the E2 protein, preferably via a peptide linker.
26. The recombinant CSFV E2 protein according to clause 25, wherein the Fc fragment comprises or consists of an amino acid sequence of SEQ ID NO: 20; and/or the peptide linker comprises or consists of an amino acid sequence selected from SEQ ID NOs: 16-19.
27. The recombinant CSFV E2 protein according to clause 26, wherein the recombinant CSFV E2 protein comprises or consists of an amino acid sequence selected from the group consisting of SEQ ID NOs: 96-101; preferably the recombinant CSFV E2 protein comprises or consists of an amino acid sequence shown by SEQ ID NO: 96.
28. A recombinant nucleic acid coding for the recombinant CSFV E2 protein according to any one of clauses 1 to 27.
29. A vector comprising the nucleic acid of clause 28.
30. A host cell comprising the nucleic acid of clause 28 or the vector of clause 29, preferably, the host cell is a mammalian cell, such as a CHO cell.
31. A method for producing the recombinant CSFV E2 protein according to any one of clauses 1-27, comprising
(i) culturing the host cell of clause 30 under conditions suitable for the expression of the CSFV E2 protein, and
(ii) isolating and optionally purifying the CSFV E2 protein.
32. A recombinant CSFV (classical swine fever virus) comprising the recombinant CSFV E2 protein of any one of clauses 1-27.
33. The recombinant CSFV of clause 32, wherein the recombinant CSFV is attenuated.
34. An immunogenic composition comprising the recombinant CSFV E2 protein according to any one of clauses 1 to 27, the recombinant nucleic acid according to clause 28, the vector according to clause 29, and/or the recombinant CSFV of clause 32 or 33.
35. The immunogenic composition according to clause 34, wherein said immunogenic composition is a vaccine, such as a marker vaccine or a DIVA (differentiation between infected and vaccinated animals) vaccine.
36. An immunogenic composition according to clause 34 or 35 for use in a method of preventing and/or treating diseases associated with CSFV in an animal, the method comprising the step of administering the immunogenic composition according to clause 34 or 35 to an animal in need thereof.
37. A method of preventing and/or treating diseases associated with CSFV in an animal, the method comprising the step of administering the immunogenic composition according to clause 34 or 35 to an animal in need thereof.
38. A method of differentiating animals infected with CSFV from animals vaccinated with the immunogenic composition of any one of clauses 34 or 35, comprising
a) obtaining a sample, and
b) testing said sample in an immuno test.
39. The method according to clause 38, wherein the immuno test comprises testing whether an antibody specifically recognizing the 6B8 epitope of the CSFV E2 protein or an antigen-binding fragment thereof can bind to the CSFV E2 protein in the sample.
40. The method according to clause 38 or 39, wherein the immuno test comprises testing whether an antibody specifically recognizing a 6B8 epitope of the CSFV E2 protein is present in the sample, and/or testing whether an antibody specifically recognizing a mutated 6B8 epitope of the recombinant CSFV E2 protein is present in the sample.
41. The method according to any one of clauses 38-40, wherein the immuno test is an EIA (enzyme immunoassay) or ELISA (enzyme linked immunosorbent assay), preferably a double competitive ELISA.
42. The method according to any one of clauses 39-41, wherein the antibody specifically recognizing the 6B8 epitope
(i) is produced by a hybridoma deposited at CCTCC under the accession number CCTCC C2018120, or
(ii) comprises a heavy chain variable region (VH) having an amino acid sequence as set forth in SEQ ID NO: 7 and a light chain variable region (VL) having an amino acid sequence as set forth in SEQ ID NO: 8, or
(iii) comprises the CDRs of the monoclonal antibody produced by a hybridoma deposited at CCTCC under the accession number CCTCC C2018120, or
(iv) comprises a VH CDR1 comprising the amino acid sequence set forth in SEQ ID NO:1, a VH CDR2 comprising the amino acid sequence set forth in SEQ ID NO:2, a VH CDR3 comprising the amino acid sequence set forth in SEQ ID NO:3, a VL CDR1 comprising the amino acid sequence set forth in SEQ ID NO:4, a VL CDR2 comprising the amino acid sequence set forth in SEQ ID NO:5, and a VL CDR3 comprising the amino acid sequence set forth in SEQ ID NO:6.
43. A kit for differentiating animals infected with CSFV from animals vaccinated with the immunogenic composition of any one of clause 34 or 35, which comprises an antibody specifically recognizing the 6B8 epitope of the CSFV E2 protein or an antigen-binding fragment thereof.
44. A method for producing the recombinant CSFV E2 protein according to any one of clauses 1 to 27, in CHO cells, said method comprises
a) adapting CHO cells to high-density suspension culture in a medium;
b) transfecting the adapted CHO cells from step a) with a mammalian expression vector comprising the nucleic acid molecule encoding the recombinant CSFV E2 protein according to any one of clauses 1 to 27,
c) culturing the transfected CHO cells from step b) under conditions suitable for the expression of the recombinant CSFV E2 protein; and
d) harvesting and optionally purifying the recombinant CSFV E2 protein.
45. A method for producing the recombinant CSFV E2 protein according to any one of clauses 1 to 27, in CHO cells, said method comprises
a) growing CHO cells to high-density suspension culture in a medium;
b) transfecting the CHO cells from step a) with a mammalian expression vector comprising the nucleic acid molecule encoding the recombinant CSFV E2 protein according to any one of clauses 1 to 27,
c) culturing the transfected CHO cells from step b) under conditions suitable for the expression of the recombinant CSFV E2 protein; and
d) harvesting and optionally purifying the recombinant CSFV E2 protein.
46. The method of clauses 44 or 45, wherein in step c), the transfected CHO cells are cultured at about 32-37° C., preferably about 32° C.
47. The method of any one of clauses 44-46, wherein in step c), the transfected CHO cells are cultured with a humidified atmosphere of about 5-8% CO₂.
48. The method of any one of clauses 44-47, wherein the recombinant CSFV E2 protein is harvested about 2-14 days post transfection, for example, about 4-12 days post transfection, or about 8-10 days post transfection.
49. The method of any one of clauses 44-48, wherein the CSFV E2 protein is further purified.
50. The method of any one of clauses 44-49, wherein the CSFV E2 protein comprises a fusion peptide and the purification of the CSFV E2 protein is done by affinity to a matrix that specifically binds to the fusion peptide.
51. The method of any one of clauses 44-50, wherein the CSFV E2 protein comprises a His-tag as a fusion peptide and the wherein CSFV E2 protein is purified by binding the CSFV E2 protein via the His-tag to Ni-NTA and release the CSFV E2 protein from the Ni-NTA.
52. The method of any one of clauses 44-50, wherein the CSFV E2 protein comprises a FLAG tag as a fusion peptide and the wherein CSFV E2 protein is purified by binding the CSFV E2 protein via the FLAG tag to a monoclonal antibody specifically binds to the FLAG tag and release the CSFV E2 protein from such monoclonal antibody.
53. The method of any one of clauses 44-50, wherein the CSFV E2 protein is linked to a Fc-fragment and the CSFV E2 protein is purified by binding the CSFV E2 protein via the Fc fragment to protein A or protein G and release the CSFV E2 protein from the protein A or protein G.

### EXAMPLES

The subsequent examples further illustrate the invention in an exemplified manner. It is understood that the invention is not limited to any of those examples as described below. A person skilled in the art understands that the performance, results and findings of these examples can be adapted and applied in a broader sense in view of the general description of the invention.

### Example 1: Construction of a Construct for the Expression of a Recombinant CSFV E2 Protein with B/C Domain Swapping Based on 6B8 Epitope

### 1.1 B/C Domains or Fragments of the B/C Domains of the E2 Proteins for Swapping

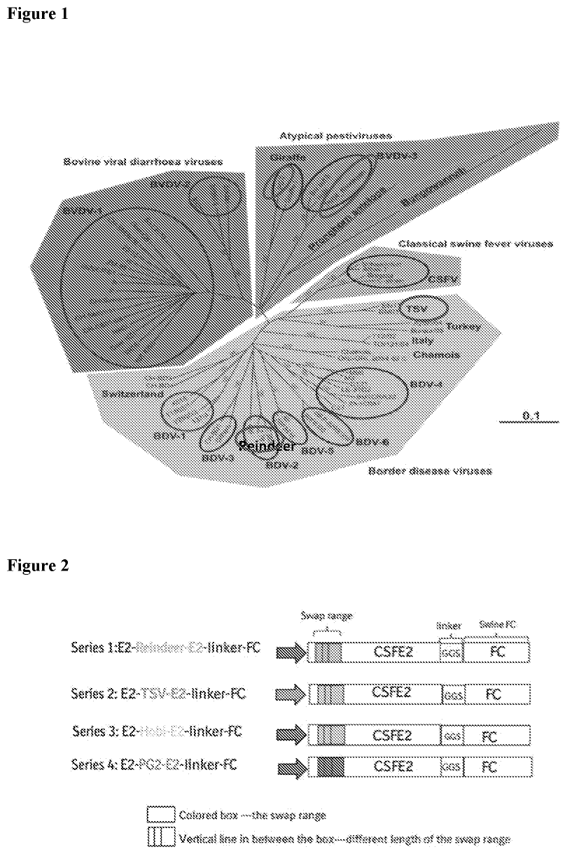

To prepare a construct for the expression of a recombinant CSFV E2 protein with B/C domain swapping based on 6B8 epitope, seven pestivirus strains other than CSFV were selected based on Ernst Peterhans's publication (FIG. 1, modified from Peterhans, et al., Cytopathic bovine viral diarrhea viruses (BVDV): emerging pestiviruses doomed to extinction. Vet. Res. Vol. 41(6).) for providing the amino acid sequences of B/C domains or fragments of the B/C domains of the E2 proteins for swapping, and the seven pestivirus strains were:
Reindeer V60 (GenBank Accession numbers of the polyprotein sequence of the virus strain is AAF02524.2; amino acid sequence of the E2 protein of the virus strain is shown by SEQ ID NO: 22)
Tunisian sheep virus 9552 (TSV9552) (GenBank Accession numbers of the polyprotein sequence of the virus strain is AAR24375.1; amino acid sequence of the E2 protein of the virus strain is shown by SEQ ID NO: 25),
PG2 (GenBank Accession numbers of the polyprotein sequence of the virus strain is AHW57610.1; amino acid sequence of the E2 protein of the virus strain is shown by SEQ ID NO: 21),
Hobi-like Th/04 KhonKaen (hereinafter named as “Hobi-like”) (GenBank Accession numbers of the polyprotein sequence of the virus strain is ACM79934.1; amino acid sequence of the E2 protein of the virus strain is shown by SEQ ID NO: 26),
Bungowannah 6778 (hereinafter named as “Bungowannah”) (GenBank Accession numbers of the polyprotein sequence of the virus strain is ABK58639.1; amino acid sequence of the E2 protein of the virus strain is shown by SEQ ID NO: 24); and
Norway rat NrPV/NYC-D23 (hereinafter named as “Norway rat”) (GenBank Accession numbers of the polyprotein sequence of the virus strain is YP_009109567; amino acid sequence of the E2 protein of the virus strain is shown by SEQ ID NO: 23).
   1.2 Construction of a Construct for the Expression of a Recombinant CSFV E2 Protein with B/C Domain Swapping Based on 6B8 Epitope

The recombinant CSFV E2 protein with B/C domain swapping based on 6B8 epitope comprises the E2 protein from CSFV QZ07 stain (SEQ ID NO: 10) and the fragment of the B/C domain of the E2 protein from the pestivirus strains disclosed in Example 1.1 for swapping. The recombinant CSFV E2 protein also optionally comprise an Fc fragment (SEQ ID NO:20) at the C terminus of the recombinant CSFV E2 protein and a peptide linker (SEQ ID NO:16) between the Fc fragment and the E2 protein.

The recombinant CSFV E2 protein also optionally comprises KARD mutations. The structures of the recombinant CSFV E2 proteins were schematically shown in FIG. 2.

Each of the amino acid sequence of the recombinant CSFV E2 protein was used for design the corresponding nucleotide sequence as a construct (each construct name was listed in Table 8), and then the designed nucleotide sequence was codon optimized and synthesized according to the instruction of CHO expression system (ExpiCHO™ Expression System, Thermo Fisher). In order to obtain a soluble and secret form E2 protein, the last 43 amino acids (aa) of E2 was deleted in final optimized sequence while the last 21 aa from El protein was added as signal peptide (SEQ ID NO: 14). Sequences synthesized each were cloned to pCDNA3.4 plasmid. A construct (QZ07E2-WT 330 FC) for expressing a wild-type E2 protein with length of 330 amino acids of QZ07E2 strain was also constructed.

Table 8 lists the virus strain names from which the amino acid sequences for swapping are derived, the amino acid sequences for swapping, original 6B8 epitopes of the virus strains, construct names, and the amino acid sequences of the recombinant CSFV E2 proteins for designing the constructs.

As for the amino acid sequences for swapping, their position numbers such as “11-39, 11-56, etc.” listed in Table 8 are determined based on the position numbers of the amino acid sequence of QZ07 CSFV E2 protein (SEQ ID NO: 10). For example, “PG2 11-39”, it refers to aligning the amino acid sequence of amino acid position 11 to position 39 of SEQ ID NO:10 (QZ07 CSFV E2 protein) with the amino acid sequence of E2 protein of the PG2 strain in a pairwise sequence alignment, then, using the corresponding aligned sequence of the of E2 protein of the PG2 strain to swap the sequence of amino acid position 11 to position 39 of SEQ ID NO:10 (QZ07 CSFV E2 protein).

As for the construct names (similar nomenclature for the constructs has the similar meanings), for example,
“QZ07E2-PG2 11-110 Chimeric FC” means the construct was designed by aligning the amino acid sequence of amino acid position 11 to position 110 of SEQ ID NO:10 (QZ07 CSFV E2 protein) with the amino acid sequence of E2 protein of the PG2 strain in a pairwise sequence alignment, then, using the corresponding aligned sequence of the of E2 protein of the PG2 strain to swap the sequence of amino acid position 11 to position 110 of SEQ ID NO:10 (i.e. “QZ07E2-PG2 11-110 Chimeric”), and using the obtained sequence for designing the corresponding nucleotide sequence of the construct. The construct also comprises the nucleotide sequence encoding an Fc fragment (SEQ ID NO:20) at the C terminus of the recombinant CSFV E2 proteins (i.e. “FC”). The nucleotide sequence encoding a peptide linker and the nucleotide sequence encoding a signal peptide also exist in the construct.
“QZ07E2-PG2 11-39 Chimeric” means the construct was designed by aligning the amino acid sequence of amino acid position 11 to position 39 of SEQ ID NO:10 (QZ07 CSFV E2 protein) with the amino acid sequence of E2 protein of the PG2 strain in a pairwise sequence alignment, then, using the corresponding aligned sequence of the of E2 protein of the PG2 strain to swap the sequence of amino acid position 11 to position 39 of SEQ ID NO:10 (i.e. “QZ07E2-PG2 11-39 Chimeric”), and using the obtained sequence for designing the corresponding nucleotide sequence of the construct. The nucleotide sequence encoding a signal peptide also exists in the construct. However, the construct does not also comprise the nucleotide sequence encoding an Fc fragment and the nucleotide sequence encoding a peptide linker.
“QZ07E2-Reindeer V60 11-56 KARD Chimeric FC” means the construct was designed by aligning the amino acid sequence of amino acid position 11 to position 56 of SEQ ID NO:10 (QZ07 CSFV E2 protein) with the amino acid sequence of E2 protein of the Reindeer V60 strain in a pairwise sequence alignment, then, using the corresponding aligned sequence of the of E2 protein of the Reindeer V60 strain to swap the sequence of amino acid position 11 to position 56 of SEQ ID NO:10 (i.e. “QZ07E2-Reindeer V60 11-56 Chimeric”), and using the obtained sequence for designing the corresponding nucleotide sequence of the construct. The construct also comprises nucleotide mutations which render KARD mutations (i.e. “KARD”). The construct also comprises the nucleotide sequence encoding an Fc fragment (SEQ ID NO:20) at the C terminus of the recombinant CSFV E2 proteins (i.e. “FC”). The nucleotide sequence encoding a peptide linker and the nucleotide sequence encoding a signal peptide also exist in the construct.

TABLE 8

| | | | | Amino acid sequences |
|---|---|---|---|---|
| | | Original 6B8 | | of the recombinant |
| Pestivirus | Sequences for | epitope of | | CSFV E2 proteins for |
| strain | swapping | the strain | Construct names | designing the constructs |
| PG2 | 11-39, 11-56, | KGES | QZ07E2-PG2 11-110 | SEQ ID NO: 58 |
| | 11-80, and | | Chimeric FC | |
| | 11-110 | | QZ07E2-PG2 11-80 | SEQ ID NO: 59 |
| | | | Chimeric FC | |
| | | | QZ07E2-PG2 11-56 | SEQ ID NO: 60 |
| | | | Chimeric FC | |
| | | | QZ07E2-PG2 11-39 | SEQ ID NO: 27 |
| | | | Chimeric | |
| Reindeer | 11-38, 11-56, | KGES | QZ07E2-Reindeer V60 | SEQ ID NO: 31 |
| V60 | 11-80, and | | 11-38 Chimeric | |
| | 11-110 | | QZ07E2-Reindeer V60 | SEQ ID NO: 63 |
| | | | 11-56 Chimeric FC | |
| | | | QZ07E2-Reindeer V60 | SEQ ID NO: 64 |
| | | | 11-80 Chimeric FC | |
| | | | QZ07E2-Reindeer V60 | SEQ ID NO: 65 |
| | | | 11-110 Chimeric FC | |
| | | | QZ07E2-Reindeer V60 | SEQ ID NO: 78 |
| | | | 11-56 KARD Chimeric FC | |
| Norway rat | 11-39 | GRNK | QZ07E2-Norway rat 11-39 | SEQ ID NO: 49 |
| | | | Chimeric FC | |
| Bungowannah | 11-38, 11-56, | KGES | QZ07E2-Bonguwannah | SEQ ID NO: 50 |
| | and 11-110 | | 11-110 Chimeric FC | |
| | | | QZ07E2-Bonguwannah | SEQ ID NO: 51 |
| | | | 11-56 Chimeric FC | |
| | | | QZ07E2-Bonguwannah | SEQ ID NO: 52 |
| | | | 11-38 Chimeric FC | |
| TSV9552 | 11-38, 11-56, | SGED | QZ07E2-TSV9552 11-90 | SEQ ID NO: 66 |
| | 11-80, 11-90, | | Chimeric FC | |
| | and 11-109 | | QZ07E2-TSV9552 11-80 | SEQ ID NO: 67 |
| | | | Chimeric FC | |
| | | | QZ07E2-TSV9552 11-109 | SEQ ID NO: 68 |
| | | | Chimeric FC | |
| | | | QZ07E2-TSV9552 11-56 | SEQ ID NO: 69 |
| | | | Chimeric FC | |
| | | | QZ07E2-TSV9552 11-38 | SEQ ID NO: 70 |
| | | | Chimeric FC | |
| | | | QZ07E2-TSV9552 11-38 | SEQ ID NO: 75 |
| | | | KARD Chimeric FC | |
| | | | QZ07E2-TSV9552 11-56 | SEQ ID NO: 77 |
| | | | KARD Chimeric FC | |
| | | | QZ07E2-TSV9552 11-109 | SEQ ID NO: 76 |
| | | | KARD- Chimeric FC | |
| Hobi-like | 11-39, 11-56, | EGTG | QZ07E2-Hobi-like 11-109 | SEQ ID NO: 53 |
| | 11-80, 11-90, | | Chimeric FC | |
| | and 11-109 | | QZ07E2-Hobi-like 11-90 | SEQ ID NO: 54 |
| | | | Chimeric FC | |
| | | | QZ07E2-Hobi-like 11-80 | SEQ ID NO: 55 |
| | | | Chimeric FC | |
| | | | QZ07E2-Hobi-like 11-56 | SEQ ID NO: 56 |
| | | | Chimeric FC | |
| | | | QZ07E2-Hobi-like 11-39 | SEQ ID NO: 57 |
| | | | Chimeric FC | |

### Example 2: Expression of the Constructs for the Recombinant CSFV E2 Proteins in CHO Cells

The constructs listed in Table 8 were transfected into CHO cells for expression of the recombinant CSFV E2 proteins. The transfection and culture were conducted following the instruction of ExpiCHO™ Expression System guide from Thermo Fisher. Finally, the cell culture media were collected by centrifuging at 4000 rpm for 15 min at 4° C. and the supernatants were collected.

The yields of the recombinant CSFV E2 proteins contained in the supernatant were detected by using SDS-PAGE and Western blot. 90 μl sample from each supernatant was mixed with 30 μl loading buffer and boiled in 95° C. for 10 min, after centrifugation, 10 μl each sample was loaded into the wells of the SDS-PAGE gel, along with a molecular weight marker. Later, the following steps were performed: running the gel at 150 V until the dye front reached the bottom of the gel (˜90 min), removing the run gel from the apparatus and placing one gel into a small tray by adding ˜20 ml staining solution and staining for >60 min with gentle shaking, destaining by H2O with gentle shaking until the gel was visibly destained (>2 hr). For western blot gels, after transferring the protein from the gel to the PVDF membrane, blocking the membrane for 1 h at room temperature or overnight at 4° C. using blocking buffer (5% skim milk in PBST), then incubating the membrane with appropriate dilutions of primary antibody WH303 (an antibody also capable of binding to the CSFV E2 protein) and 6B8 in blocking buffer, washing the membrane in three washes of PBST, 5 min each, incubating the membrane with the recommended dilution of conjugated secondary antibody in blocking buffer at room temperature for 1 h and then washing the membrane in three washes of PBST, 5 min each and subjecting to signal development.

17 supernatants of the recombinant CSFV E2 protein were randomly chosen as candidates for further western blotting experiment, or DIVA and Efficacy evaluation. The supernatants were those prepared from the expressions of the following constructs: QZ07E2-PG2 11-39 Chimeric FC, QZ07E2-PG2 11-56 Chimeric FC, QZ07E2-Reindeer V60 11-38 Chimeric FC, QZ07E2-Reindeer V60 11-56 Chimeric FC, QZ07E2-Reindeer V60 11-56 KARD Chimeric FC, QZ07E2-Reindeer V60 11-80 Chimeric FC, QZ07E2-Reindeer V60 11-110 Chimeric FC, QZ07E2-TSV 11-38 Chimeric FC, QZ07E2-TSV9552 11-38 KARD Chimeric FC, QZ07E2-TSV9552 11-56 Chimeric FC, QZ07E2-TSV9552 11-56 KARD Chimeric FC, QZ07E2-TSV9552 11-80 Chimeric FC, QZ07E2-TSV9552 11-109 Chimeric FC, QZ07E2-Hobi-like 11-39 Chimeric FC, QZ07E2-Hobi-like 11-56 Chimeric FC, QZ07E2-Hobi-like 11-109 Chimeric FC, and QZ07E2-Bungowannah 11-38 Chimeric FC. The supernatant prepared from the expression of the construct QZ07E2-WT 330 FC was also prepared and used for further tests.

### Example 3: Test for the Inhibition of 6B8 Antibody Binding

The objective of this Example was to evaluate in vitro whether the swapping of the B/C domain of the recombinant CSFV E2 protein or the swapping of the B/C domain of the recombinant CSFV E2 protein together with KARD mutations can block the binding of 6B8 antibody to the recombinant CSFV E2 protein.

The above 17 candidates listed in Example 2 were subject to western blotting experiment to determine the inhibition of 6B8 antibody binding, which is a key feature for DIVA. Detailed steps for western blotting experiment can be referred to Example 2.

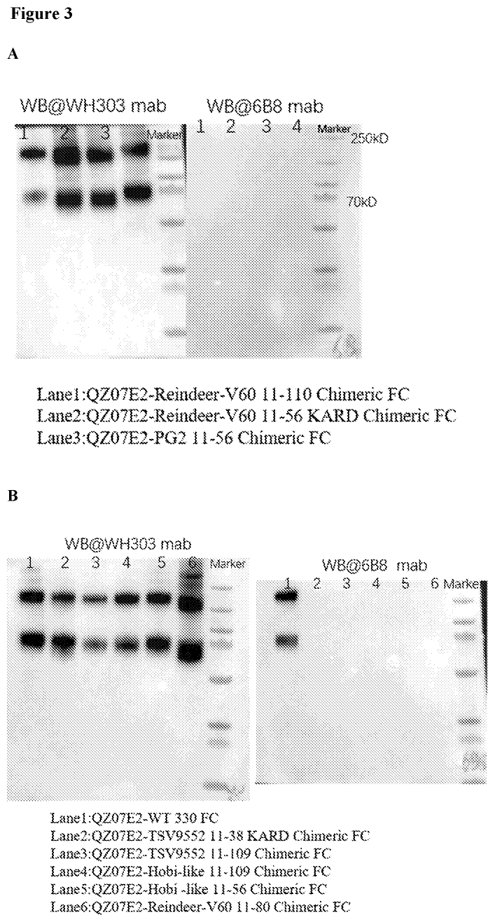

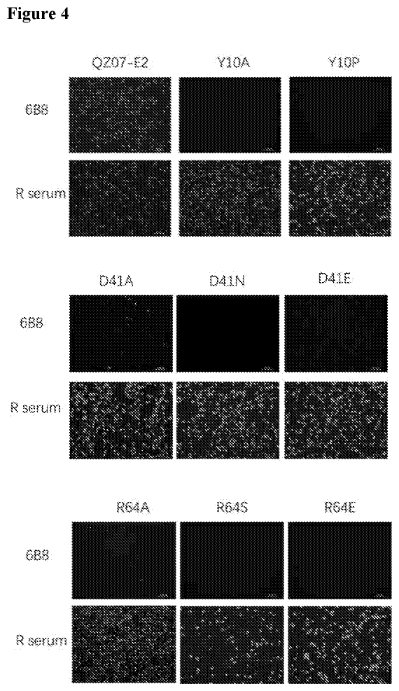

All supernatants showed no antibody binding to 6B8 antibody, while showing binding to WH303 antibody. FIGS. 3A and 3B showed the western blot results of QZ07E2-Reindeer V60 11-110 Chimeric FC, QZ07E2-Reindeer V60 11-56 KARD Chimeric FC, QZ07E2-PG2 11-56 Chimeric FC, QZ07E2-TSV9552 KARD Chimeric FC, QZ07E2-TSV9552 11-109 Chimeric FC, QZ07E2-Hobi-like 11-109 Chimeric FC, QZ07E2-Hobi-like 11-56 Chimeric FC and QZ07E2-Reindeer V60 11-80 Chimeric FC. It can be seen from FIGS. 3A and 3B that lanes 1-3 of FIG. 3A and lane 2-6 of FIG. 3B showed no 6B8 antibody binding; In lane 1 of FIG. 3B the supernatant from wildtype E2 protein of QZ07E2 strain was added, and 6B8 antibody binding is shown; and all the bands in the lanes can bind to the WH303 antibody which means all the proteins were well expressed and existed. It was demonstrated that the disabled reactivity of these proteins with 6B8 antibody was directly caused by the swapping of B/C domain with or without KARD mutations.

### Example 4: Efficacy Evaluation

The objective of this Example was to evaluate the efficacy of the recombinant E2 proteins by using compositions prepared from the supernatants of Example 2 in an exemplary manner. Eight supernatants of Example 3 were randomly chosen as candidates.

Briefly, a total of 60 commercial mix breed piglets at the age of 21+7 days old piglets (3-week old) were assigned into 11 groups:
Group 1 containing 5 piglets served as the E2 control without DIVA feature;
Groups 2-9 containing 5 piglets for each group were used for efficacy evaluation;
Group 10 containing 5 piglets served as challenge control; and
Group 11 containing 5 piglets served as strict (negative) control.

The name of each group of the recombinant E2 protein and efficacy evaluation were listed in Table 9. The supernatants prepared from Example 2 were each mixed with Seppic ISA 206 adjuvant so as to prepare immunogenic compositions. Between 55 and 65 μg of non-purified recombinant E2 protein was contained in one dose of a composition (2 ml) for administration and used in such experiment. In the composition, the weight ratio of the recombinant E2 protein contained in the supernatant and the Seppic ISA 206 adjuvant is about 1:1.

TABLE 9

| Group | | ug/dose |
|---|---|---|
| 1 | QZ07E2-WT 330 FC | 64.62 |
| 2 | QZ07E2-Reindeer V60 11-56 KARD Chimeric FC | 55.43 |
| 3 | QZ07E2-Reindeer V60 11-80 Chimeric FC | 62.52 |
| 4 | QZ07E2-Reindeer V60 11-110 Chimeric FC | 50.94 |
| 5 | QZ07E2-PG2 11-56 Chimeric FC | 51.15 |
| 6 | QZ07E2-Hobi-like 11-56 Chimeric FC | 61.99 |
| 7 | QZ07E2-Hobi-like 11-109 Chimeric FC | 62.57 |
| 8 | QZ07E2-TSV9552 11-109 Chimeric FC | 63.22 |
| 9 | QZ07E2-TSV9552 11-38 KARD Chimeric FC | 61.59 |
| 10 | 2 mL PBS + Adjuvant (Seppic ISA 206) | Not applicable |
| 11 | Strict Control | Not applicable |

On Day 0, piglets in Groups 1-9 were inoculated (IM) with 2 mL Seppic ISA 206 adjuvanated compositions per piglet, respectively. Group 10 was inoculated (IM) with 2 mL PBS+Adjuvant (Seppic ISA 206) on Day 0, served as challenge control. Piglets in groups 1-10 were inoculated (IM) with infectious CSFV Shimen strain (provided by Institute of Military Veterinary Medicine, Academy of Military Sciences) at dose ≥10⁵ MLD/mL on Day 21. All piglets were clinical healthy and free for CSFV and PRRSV antibodies and free of antigen including BVDV, PRV on Day 0. All piglets were healthy at the time of immunization.

Serum samples were collected every 7 days starting from-Day 0. On Days 21, 24, 28, 31 and 37 (days post challenge (DPC) 0, 3, 7, 10, 16), whole blood samples of all piglets were collected. The main parameter for determining vaccine efficacy was mortality, and mortality is associated with severe classic swine fever (CSF) infection.

As shown in Table 10 below, piglets were all dead in challenge control group (Group 10). As for groups 4, 5 and 8, they were all showed 20% mortality which is still much better than the challenge group, and the others all conferred 100% mortality protection against the Shimen strain challenge. The results showed that the replacement with corresponding fragments from other pestiviruses and the incorporation of amino acid mutations in the recombinant E2 proteins did not affect the vaccine efficacy. Thus, all vaccine candidates showed vaccine efficacy.

The results of efficacy evaluation for all the candidates were summarized in Table 10 below.

TABLE 10

| | Group | Mortality (%) |
|---|---|---|
| 1 | QZ07E2-WT 330 FC | 0 |
| 2 | QZ07E2-Reindeer V60 11-56 KARD Chimeric FC | 0 |
| 3 | QZ07E2-Reindeer V60 11-80 Chimeric FC | 0 |
| 4 | QZ07E2-Reindeer V60 11-110 Chimeric FC | 20 |
| 5 | QZ07E2-PG2 11-56 Chimeric FC | 20 |
| 6 | QZ07E2-Hobi-like 11-56 Chimeric FC | 0 |
| 7 | QZ07E2-Hobi-like 11-109 Chimeric FC | 0 |
| 8 | QZ07E2-TSV9552 11-109 Chimeric FC | 20 |
| 9 | QZ07E2-TSV9552 11-38 KARD Chimeric FC | 0 |
| 10 | Challenge control | 100 |
| 11 | Strict control | 0 |

### Example 5: Immune Response Evaluation

In this Example, the recombinant E2 proteins prepared from the constructs of Table 9 (groups 2-9) were subject to an immune response evaluation. The preparation methods of the immunogenic compositions of these recombinant E2 proteins and the subsequent experimental design including the grouping of piglets and dosage of the immunization, etc. were the same as the method as described in Example 4. Piglets in groups 1-10 were challenged by inoculating (IM) with infectious CSFV Shimen strain (provided by Institute of Military Veterinary Medicine, Academy of Military Sciences) at dose ≥10⁵ MLD/mL on Day 21.

Serum samples were collected every 7 days starting from Day 0 to 16 days post challenge (DPC16). The main parameter for evaluating the antibody response after vaccination was antibody blocking rate which reflects the antibody level of the vaccinated piglet. The value of the antibody blocking rate was determined by following the protocols of IDEXX CSFV Ab ELISA assay kit (IDEXX Laboratories. Inc).

Table 11 shows the average antibody blocking rate (%) of the vaccinated piglets in each group at different days.

TABLE 11

| | | Average Antibody Blocking Rate (%) of Different Days | | | | | | | |
|---|---|---|---|---|---|---|---|---|---|
| Group | | D 0 | D 7 | D 14 | D 21 | DPC3 | DPC7 | DPC10 | DPC16 |
| 1 | QZ07E2-WT 330 FC | 12.72 | 3.32 | 46.85 | 79.87 | 83.98 | 89.30 | 89.63 | 86.94 |
| 2 | QZ07E2-Reindeer V60 11-56 KARD Chimeric FC | 15.41 | 19.05 | 57.94 | 82.62 | 87.48 | 93.12 | 93.46 | 91.18 |
| 3 | QZ07E2-Reindeer V60 11-80 Chimeric FC | 18.72 | 13.92 | 58.78 | 75.65 | 81.22 | 91.31 | 92.13 | 89.31 |
| 4 | QZ07E2-Reindeer V60 11-110 Chimeric FC | 13.93 | 4.38 | 41.56 | 65.70 | 71.36 | 89.41 | 89.80 | 86.41 |
| 5 | QZ07E2-PG2 11-56 Chimeric FC | 16.44 | 26.87 | 61.41 | 82.01 | 85.68 | 93.50 | 93.19 | 92.19 |
| 6 | QZ07E2-Hobi-like 11-56 Chimeric FC | 14.90 | 8.16 | 63.18 | 79.03 | 83.63 | 93.22 | 92.00 | 92.16 |
| 7 | QZ07E2-Hobi-like 11-109 Chimeric FC | 12.88 | 2.15 | 49.90 | 68.99 | 74.88 | 88.33 | 87.50 | 87.21 |
| 8 | QZ07E2-TSV9552 11-109 Chimeric FC | −9.05 | −3.85 | 42.02 | 53.27 | 54.14 | 83.48 | 81.62 | 86.90 |
| 9 | QZ07E2-TSV9552 11-38 KARD Chimeric FC | 10.10 | 7.46 | 67.07 | 77.04 | 77.46 | 92.89 | 91.68 | 92.15 |
| 10 | Challenge Control | −10.19 | −5.55 | −0.12 | 3.01 | −6.82 | −1.68 | 21.76 | Death |
| 11 | Strict Control | −13.10 | −9.30 | −2.53 | −1.23 | −2.05 | −4.64 | −2.72 | 4.13 |

As shown in Table 11, except from the two control groups 10-11, the antibody blocking rates of the vaccinated groups 1-9 became positive (blocking rate >40%, which means that a large amount of antibodies are present in the serum sample) on D14 and then rapidly rised until D21, and on DPC7, the antibody blocking rates of the vaccinated groups 1-9 reached to the peak values. Moreover, the antibody blocking rates of the vaccinated groups 2-9 were comparable to that of the vaccinated group 1 from DPC7 to DPC16. These results showed that the recombinant E2 proteins prepared from the constructs listed in Table 9 successfully elicited the antibody response in vaccinated piglets, and the replacement with the corresponding fragment from other pestiviruses and incorporation of the amino acid mutations did not affect the immunogenicity of the vaccine.

### Example 6: In Vivo DIVA Test

In this Example, the recombinant E2 proteins prepared from the constructs of Table 9 (groups 2-9) were subject to an in vivo DIVA test. The preparation methods of the immunogenic compositions of these recombinant E2 proteins and the subsequent experimental design including the grouping of piglets and dosage of the immunization, etc. were the same as the method as described in Example 4. Piglets in groups 1-10 were challenged by inoculating (IM) with infectious CSFV Shimen strain (provided by Institute of Military Veterinary Medicine, Academy of Military Sciences) at dose ≥10⁵ MLD/mL on Day 21. Serum samples were collected on Day 21 (D21) and 16 days post challenge (DPC16) for DIVA test.

Serum samples were subject to a double competitive ELISA (dcELISA) method to detect the antibody response against 6B8 antibody which was used as a DIVA marker. The dcELISA method was performed according to Bruderer U., J Immunol Methods. 2015 May; 420:18-23, and 6B8 monoclonal antibody was used as a competitive antibody in the dcELISA method. The value for evaluating the antibody level of the vaccinated piglet was an antibody blocking rate, which was obtained by measuring the mean absorbance value of the serum sample and the strict control at OD450 for each group, and then calculating the antibody blocking rate by dividing the difference value between OD450 value of the strict control and OD450 value of the serum sample by OD450 value of the strict control. The test serum sample is positive (6B8 antibodies are present) if it shows a blocking percentage >30%. The test serum sample is negative (6B8 antibodies are absent) if it shows a blocking percentage≤30%.

Table 12 shows the average antibody blocking rate (%) of the vaccinated piglets in each group on D21 and DPC16.

TABLE 12

| | | Average | Average |
|---|---|---|---|
| | | Antibody | Antibody |
| | | Blocking | Blocking |
| | | Rate % | Rate % |
| | Group | on D 21 | on DPC16 |
| 1 | QZ07E2-WT 330 FC | 65.04 | 92.48 |
| 2 | QZ07E2-Reindeer V60 11-56 KARD | 25.186 | 55.31 |
| | Chimeric FC | | |
| 3 | QZ07E2-Reindeer V60 11-80 Chimeric FC | 24.758 | 79.386 |
| 4 | QZ07E2-Reindeer V60 11-110 Chimeric FC | 17.048 | 84.0675 |
| 5 | QZ07E2-PG2 11-56 Chimeric FC | 27.796 | 62.1 |
| 6 | QZ07E2-Hobi-like 11-56 Chimeric FC | 16.774 | 78.03 |
| 7 | QZ07E2-Hobi-like 11-109 Chimeric FC | 9.788 | 61.654 |
| 8 | QZ07E2-TSV9552 11-109 Chimeric FC | 2.596 | 66.8525 |
| 9 | QZ07E2-TSV9552 11-38 KARD | 7.915 | 74.67 |
| | Chimeric FC | | |
| 10 | Challenge control | −4.674 | Not |
| | | | applicable |
| 11 | Strict control | 3.243 | 3.656 |

As shown in Table 12, except from group 1, the 6B8 antibody blocking rates of the vaccinated groups 2-9 were negative on D21 after vaccination, and the 6B8 antibody blocking rates of all groups 1-9 became positive on DPC16. These results showed that the recombinant E2 proteins comprising the chimeric E2 fragments from other pestiviruses have the DIVA capability, and further incorporation of the amino acid mutations can ensure the DIVA capability. Thus, the recombinant E2 proteins prepared from the constructs listed in Table 9 have the DIVA capability.

### Example 7: Identification of Critical Amino Acid Positions for 6B8 Binding in E2 Protein

In WO2020211802A (the entire contents of WO2020211802A are incorporated by reference herein), amino acid positions 14, 22, 24, and 24/25 in CSFV E2 protein had already been identified as critical for 6B8 monoclonal antibody binding. Specifically, a substitution of E or G to R or K at amino acid position 24 of the E2 protein, a substitution of E or G to R or K at amino acid position 24 and a substitution of G to D at amino acid position 25 of the E2 protein, a substitution of S to K, Q or R at amino acid position 14 of the E2 protein, and/or a substitution of G to A, R, Q, or E at amino acid position 22 of the E2 protein were demonstrated as specifically inhibiting the binding of 6B8 antibody to the CSFV E2 protein.

The following example is to identify additional critical amino acid positions for 6B8 binding in E2 protein.

In this Example, amino acid positions 10, 41 and 64 in E2 were further identified as additional critical positions for 6B8 mAb binding.

Mutant QZ07-E2 proteins contain Y10A, Y10P, D41A, D41N, D41E, R64A, R64S, or R64E (amino acid sequences shown in SEQ ID NOs: 79-86, respectively; and the codon-optimized coding sequences were shown in SEQ ID NOs: 87-94, respectively) respectively. The codon-optimized coding sequence of QZ07-E2 protein without mutations was shown in SEQ ID NO: 95. Each codon-optimized sequence was synthesized and then was cloned to pVL1393 shuttle plasmid by BamH I and EcoR I to complete the pVL1393-shuttle plasmid for further co-transfection. In order to obtain soluble and secret form E2 protein, the last 42 amino acids of E2 was deleted in final sequence while the last 16 amino acids from El protein was added as signal peptide. CHO expression system (ExpiCHO™ Expression System, Thermo Fisher) was used for further expression of the proteins. Immunoinfluoscent Assay (IFA) for determining the binding of 6B8 mAb to the mutant QZ07-E2 proteins with a mutated 6B8 epitope. The detailed methods of constructing and expressing the mutant QZ07-E2 proteins, together with the IFA test can be referred to WO2020211802A.

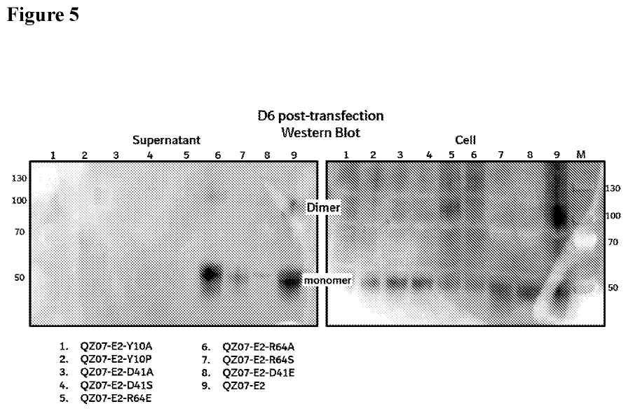

The results are shown in FIG. 4, and the results indicate that substitutions Y10A, Y10P, D41A, D41N, D41E, R64A, R64S, and R64E can inhibit 6B8 mAb binding respectively. Expression of mutant QZ07-E2 proteins containing Y10A, Y10P, D41A, D41N, D41E, R64A, R64S, or R64E was detected by Western Blotting. The results are shown in FIG. 5, and the results indicate that mutations R64A and R64S only slightly affected the protein expression secretion in CHO cell system.

### Example 8: Identification of Additional Amino Acid Mutations for Inhibiting 6B8 Binding in E2 Protein

In this Example, more mutations at amino acid positions 14, 22, 24, 25 and 64 in E2 were identified as additional critical for inhibiting 6B8 mAb binding.

Mutant QZ07-E2 proteins containing various mutations at positions 14, 22, 24, 25 and 64 respectively were designed, and the corresponding coding sequences of the mutant QZ07-E2 proteins were codon optimized and synthesized according the instruction of CHO expression system (ExpiCHO™ Expression System, Thermo Fisher). In order to obtain soluble and secret form E2 protein, the last 43 amino acids of E2 was deleted in final optimized sequence while the last 21 amino acids from E1 protein was added as signal peptide. CHO expression system (ExpiCHO™ Expression System, Thermo Fisher) was used for further expression of the mutant QZ07-E2 proteins. The detailed methods of constructing and expressing the mutant QZ07-E2 proteins followed above Example 1 and Examples 1-3 of WO2020211802A.

Some mutants including Mutant QZ07-E2-Fc-R64D, Mutant QZ07-E2-Fc-R64H, Mutant QZ07-E2-Fc-R64T, Mutant QZ07-E2-Fc-R64G, and Mutant QZ07-E2-Fc-R64K were prepared by further linking C terminus of mutant E2 to an Fc fragment for enhancing the protein expression. A peptide linker was also added between E2 protein and Fc fragment. The codon-optimized sequences for the mutant QZ07-E2-Fc proteins were cloned to pCDNA3.4 plasmids.

The transfection and culture of the recombinant pCDNA3.4 plasmids were also done by following the instruction of ExpiCHO™ Expression System guide from Thermo Fisher.

On the day prior to transfection (Day −1), the ExpiCHO-S™ culture was splitted to a final density of 3×10⁶-4×10⁶ viable cells/mL, and the cells were allowed to grow overnight. On the day of transfection (Day 0), viable cell density and percent viability were determined. When the cells reached a density of approximately 7×10⁶-10×10⁶ viable cells/mL with a viability of 95-99%, the cells were diluted to a final density of 6×10⁶ viable cells/mL with fresh ExpiCHO™ Expression Medium. The flasks were swirled gently to mix the cells. ExpiFectamine™ CHO/plasmid DNA complexes using cold reagents (4° C.) were prepared. Reagents from refrigeration were simply removed, and DNA complexation was commenced. The following steps comprised: a) gently inverting the ExpiFectamine™ CHO Reagent bottle 4-5 times to mix, b) diluting plasmid DNA with cold OptiPRO™ medium, and mixing by swirling the tube and/or by inversion, c) diluting ExpiFectamine™ CHO Reagent with OptiPRO™ medium, mixing by swirling the tube and/or by inversion or gentel pipetting 2-3 times, mixing by swirling the tube or by inversion, incubating ExpiFectamine™ CHO/plasmid DNA complexes (from Step d) at room temperature for 1-5 minutes, and then slowly transferring the solution to the shaker flask, swirling the flask gently during addition. Day 1, On the day after transfection (Day 1, 18-22 hours post-transfection), ExpiFectamine™ CHO Enhancer and ExpiCHO™ Feed were added to the flask (adding volume according to the guide), followed by gently swirling the flask during addition. The flask was transferred to a 32° C. incubator with a humidified atmosphere of 5% CO2 in air with shaking and was cultured for 6-10 days. Cell culture medium was collected by centrifuge at 4000 rpm for 15 min at 4° C. and the supernatant was collected.

The purification of mutant QZ07-E2-Fc proteins was performed by using Protein A Agarose from Thermo, respectively. The purification solution comprises 1 M Tris buffer (pH 9.0). The purification process comprises the steps of equilibrating the Protein Agarose and all buffers to room temperature; carefully packing the column with 0.5 ml (for QZ07-E2-Fc mutations, such as mutation at position 64) of resin slurry, following the instructions provided with the columns; equilibrating the column by adding 5 ml of Binding Buffer and allowing the solution to drain through the column; diluting sample at 1:2 with Binding Buffer before application to the Protein A column to maintain the proper ionic strength and pH for optimal binding; applying the diluted sample to the column and allow it to flow completely into the resin; washing the Protein A column with 10 ml of the Binding Buffer; eluting antibodies with 5 ml of Elution Buffer and collect the fraction; immediately adjusting eluted fractions to physiologic pH by adding 100 μl of the Neutralization Buffer 1 ml of elute; transferring the elution into centrifugal filters, and adding 6 ml of PBS; placing the tube at centrifuge, 4000 rpm for 15 min; washing the tube using 10 ml of PBS; repeat the centrifugation step to concentrate the sample into 1-2 ml; using Qubit Kit to measure the purified-buffer exchanged samples; and confirming the product by SDS-PAGE and Western Blot analysis.

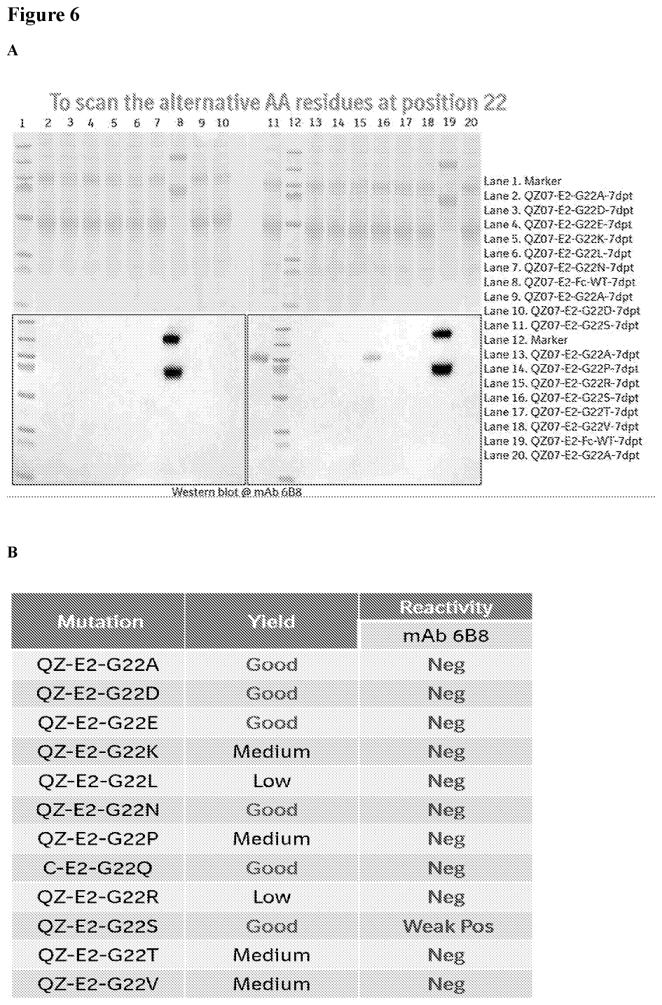

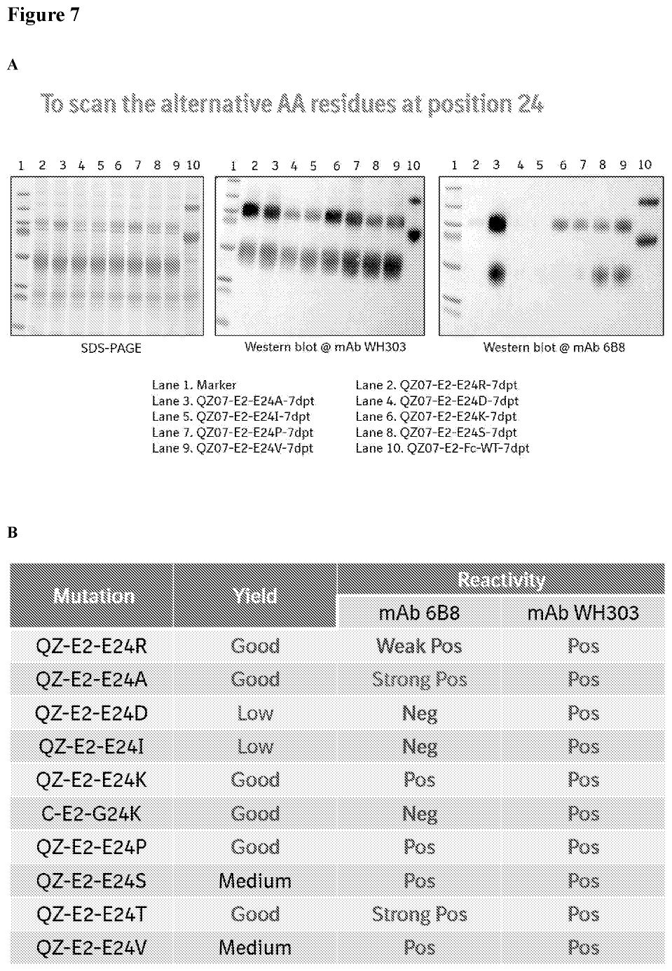

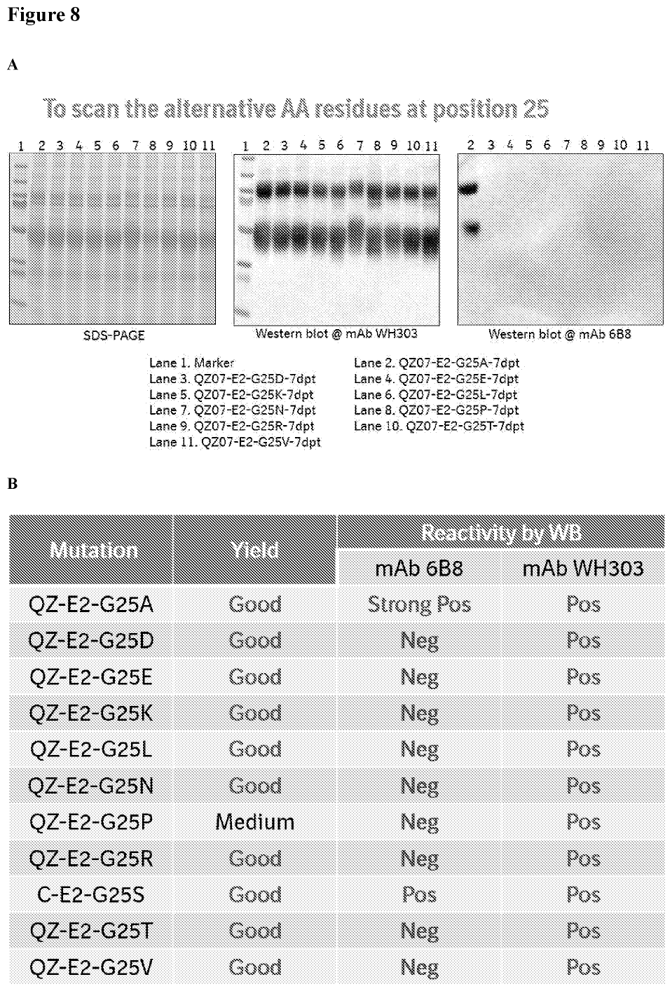

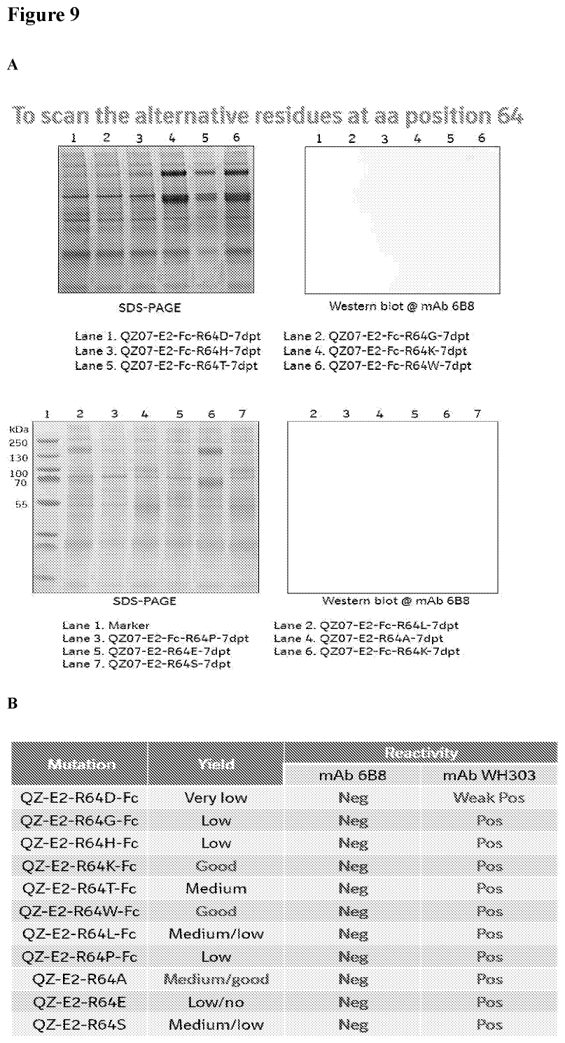

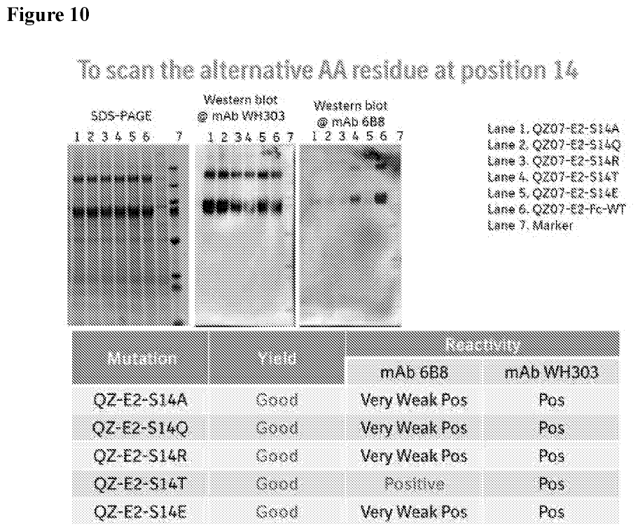

The purification products of mutant QZ07-E2 proteins and mutant QZ07-E2-Fc proteins were confirmed by SDS-PAGE and Western Blot analysis. The results are shown in FIG. 6-10.

For amino acid position 22, G22A, G22Q, G22D, G22E, G22N and G22S can be candidate substitutions in terms of both yield and reactivity with mAb 6B8.

For amino acid position 24, G24R still have weak reactivity with mAb 6B8. G24D and G24I mutants cannot react with mAb 6B8, although the yield is low.

For amino acid position 25, G25D, G25K, G25L, G25N, G25R, G25T and G25V can be candidate substitutions in terms of both yield and reactivity with mAb 6B8.

For amino acid position 64, R64A, R64K and R64W are the candidate mutants in terms of yield and reactivity with mAb 6B8.

For amino acid position 14, S14A, S14Q, S14R, S14E showed very weak reactivity with mAb 6B8, and the mutations are suitable candidate substitutions in terms of both yield and reactivity with mAb 6B8 The very weak reactivity with mAb 6B8 allows to define a cut-off value with a clear distinction between an mAb 6B8 positive- and mAb 6B8 negative-reactivity.

### Example 9: Construction and Expression of Additional Constructs for the Expression of a Recombinant CSFV E2 Protein with B/C Domain Swapping and One Single Mutation

In this Example, six additional constructs were prepared, and then expressed in CHO cells according to the method as described in Examples 1 and 2. The constructs were named as QZ07E2-Hobi-like 11-109 Chimeric E14K FC, QZ07E2-Hobi-like 11-109 Chimeric R64K FC, QZ07E2-Hobi-like 11-109 Chimeric T24R FC, QZ07E2-Hobi-like 11-109 Chimeric G25D FC, QZ07E2-Hobi-like 11-109 Chimeric G25N FC, and QZ07E2-Hobi-like 11-109 Chimeric G25R FC.

Construct QZ07E2-Hobi-like 11-109 Chimeric E14K FC was prepared by aligning the amino acid sequence of amino acid position 11 to position 109 of SEQ ID NO:10 (QZ07 CSFV E2 protein) with the amino acid sequence of E2 protein of the Hobi-like strain in a pairwise sequence alignment, then, using the corresponding aligned sequence of the E2 protein of the Hobi-like strain to swap the sequence of amino acid position 11 to position 109 of SEQ ID NO:10, and using the obtained amino acid sequence to design the corresponding nucleotide sequence of the construct. Nucleotide mutations which render E14K mutations were also introduced into the corresponding nucleotide sequence of the construct. A nucleotide sequence encoding an Fc fragment (SEQ ID NO:20), and a nucleotide sequence encoding a peptide linker (SEQ ID NO:16) and a nucleotide sequence encoding a signal peptide (SEQ ID NO:14) were also added to the construct. Other constructs were prepared in the same way.

The recombinant CSFV E2 proteins expressed by these constructs have a B/C domain swapping and one single amino acid mutation in a position which had been identified in Examples 7-8 as being critical for inhibiting 6B8 mAb binding. The supernatants containing the recombinant CSFV E2 proteins were obtained and collected according to the method as described in Example 2.

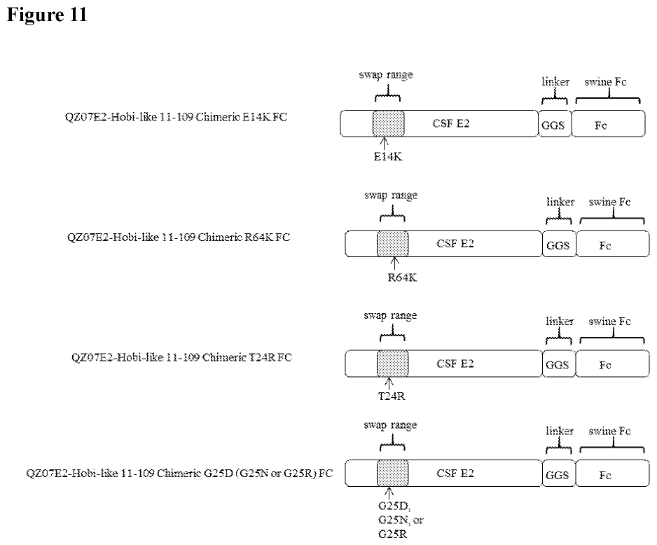

The structures of the recombinant CSFV E2 proteins expressed from these constructs of were schematized in FIG. 11. The nucleotide sequences of these constructs for expressing the recombinant CSFV E2 proteins, and the amino acid sequences of the recombinant CSFV E2 proteins expressed from the constructs (signal peptides were removed after the expression) were listed in Table 13.

TABLE 13

| | Amino acid | Nucleotide |
|---|---|---|
| | sequences of | sequences of |
| | the recombinant | these constructs |
| | CSFV E2 proteins | for expressing the |
| | expressed from | recombinant CSFV |
| Construct names | the constructs | E2 proteins |
| QZ07E2-Hobi-like 11-109 | SEQ ID NO: 96 | SEQ ID NO: 102 |
| Chimeric E14K FC | | |
| QZ07E2-Hobi-like 11-109 | SEQ ID NO: 97 | SEQ ID NO: 103 |
| Chimeric R64K FC | | |
| QZ07E2-Hobi-like 11-109 | SEQ ID NO: 98 | SEQ ID NO: 104 |
| Chimeric T24R FC | | |
| QZ07E2-Hobi-like 11-109 | SEQ ID NO: 99 | SEQ ID NO: 105 |
| Chimeric G25D FC | | |
| QZ07E2-Hobi-like 11-109 | SEQ ID NO: 100 | SEQ ID NO: 106 |
| Chimeric G25N FC | | |
| QZ07E2-Hobi-like 11-109 | SEQ ID NO: 101 | SEQ ID NO: 107 |
| Chimeric G25R FC | | |

### Example 10: In Vitro Test for the Inhibition of 6B8 Antibody Binding

In this example, the supernatants prepared from the expression of the six constructs of Example 9 together with QZ07E2-WT 330 FC and QZ07E2-Hobi-like 11-109 Chimeric FC were subject to a western blotting experiment so as to determine the inhibition of 6B8 antibody binding. The western blotting experiment was performed according to the methods as described in Examples 2-3.

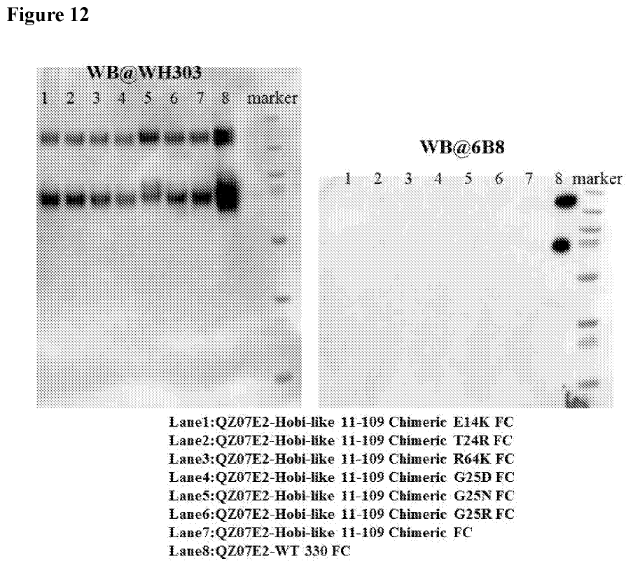

FIG. 12 shows the Western blot results of the supernatants prepared from the expression of these constructs. Binding blots were present in lanes 1-8 of FIG. 12 (left figure), which indicates that the supernatants prepared from all of the constructs can react with WH303 monoclonal antibody. Binding blots were not present in lanes 1-7 of FIG. 12 (right figure), which indicates that the supernatants prepared from the corresponding constructs can not react with 6B8 monoclonal antibody. These results showed that the disabled reactivity of these proteins with 6B8 monoclonal antibody was directly caused by the swapping of B/C domain and the single mutation, which suggests that the recombinants E2 protein expressed from these constructs could be used for the differentiation of vaccinated animals from those infected by wild type field strains.

### Example 11: Immune Response Evaluation

In this Example, the recombinant E2 proteins prepared from the constructs of Table 13 were subject to an immune response evaluation. A total of 39 commercial mix breed piglets (from Rugao Boyi Animal Husbandry Co., Ltd., NanTong, China) at the age of 21+7 days old piglets (3-week old) were assigned into 9 groups:
Groups 1-7 containing 5 piglets for each group were used for immune response evaluation;
Group 8 containing 2 piglets served as the E2 control without DIVA feature;
Group 9 containing 2 piglets served as strict (negative) control.

The supernatants prepared from the expression of the six constructs of Example 8 together with QZ07E2-WT 330 FC and QZ07E2-Hobi-like 11-109 Chimeric FC were each mixed with Seppic ISA 206 adjuvant so as to prepare immunogenic compositions. In each of the immunogenic composition, the weight ratio of the recombinant E2 protein contained in the supernatant and the Seppic ISA 206 adjuvant is about 1:1. Each piglet was administrated to one dose of the immunogenic composition (2 ml). The amount of the recombinant E2 protein contained in one dose of the immunogenic composition was listed in Table 14.

TABLE 14

| Group | | μg per dose |
|---|---|---|
| 1 | QZ07E2-Hobi-like 11-109 Chimeric E14K FC | 55.26 |
| 2 | QZ07E2-Hobi-like 11-109 Chimeric R64K FC | 58.41 |
| 3 | QZ07E2-Hobi-like 11-109 Chimeric T24R FC | 54.77 |
| 4 | QZ07E2-Hobi-like 11-109 Chimeric G25D FC | 49.39 |
| 5 | QZ07E2-Hobi-like 11-109 Chimeric G25N FC | 63.29 |
| 6 | QZ07E2-Hobi-like 11-109 Chimeric G25R FC | 54.99 |
| 7 | QZ07E2-Hobi-like 11-109 Chimeric FC | 49.39 |
| 8 | QZ07E2-WT 330 FC | 53.03 |
| 9 | 2 mL PBS + Adjuvant (Seppic ISA 206) | Not applicable |

On Day 0, each piglet in Groups 1-8 was inoculated (IM) with 2 mL of the composition. Group 9 was inoculated (IM) with 2 mL PBS+Adjuvant (Seppic ISA 206) on Day 0, served as strict control. On Day 21 Piglets in groups 1-9 were boosted with the same antigen as used on Day 0. All piglets were clinical healthy and free for CSFV and PRRSV antibodies and free of antigen including BVDV, PRV on Day 0. All piglets were healthy at the time of immunization.

Serum samples were collected every 7 days starting from Day 0 to Day 56. The main parameter for evaluating the antibody response after vaccination was called antibody blocking rate which reflects the antibody level of the vaccinated piglet. The value of the antibody blocking rate was determined by following the protocols of IDEXX CSFV Ab ELISA assay kit (IDEXX Laboratories. Inc).

Table 15 shows the average antibody blocking rate (%) of the vaccinated piglets in each group at different days. As shown in Table 15, except from the control group 9, the antibody blocking rates of the vaccinated groups 1-8 became positive (blocking rate >40%) on D21, and after boost immunization, the antibody blocking rates of the vaccinated groups 1-8 continuously increased and then reached to the peak values. Moreover, from D21 to D56, the antibody blocking rates of the vaccinated groups 1-7 were comparable to that of the vaccinated group 8. These results showed that the replacement with the corresponding fragment from other pestiviruses and incorporation of the amino acid mutations did not affect the immunogenicity of the vaccine, and the recombinant E2 proteins prepared from the constructs successfully elicited good antibody response in vaccinated piglets.

TABLE 15

| | | Average Antibody Blocking Rate (%) of Different Days Post Vaccination | | | | | | | | |
|---|---|---|---|---|---|---|---|---|---|---|
| Group | | D 0 | D 7 | D 14 | D 21 | D 28 | D 35 | D 42 | D 49 | D 56 |
| 1 | QZ07E2-Hobi-like 11-109 Chimeric T24R FC | 8.56 | 5.57 | 51.19 | 66.42 | 77.56 | 81.46 | 85.95 | 79.42 | 84.70 |
| 2 | QZ07E2-Hobi-like 11-109 Chimeric E14K FC | 5.34 | 4.94 | 43.55 | 61.68 | 80.40 | 73.45 | 85.11 | 79.23 | 86.47 |
| 3 | QZ07E2-Hobi-like 11-109 Chimeric R64K FC | 4.01 | 3.11 | 24.40 | 49.09 | 72.94 | 74.62 | 80.85 | 76.17 | 83.19 |
| 4 | QZ07E2-Hobi-like 11-109 Chimeric G25N FC | 5.65 | 9.90 | 39.31 | 46.65 | 70.79 | 72.13 | 78.22 | 76.70 | 81.93 |
| 5 | QZ07E2-Hobi-like 11-109 Chimeric G25R FC | 7.17 | 7.24 | 43.33 | 52.25 | 77.73 | 81.29 | 85.10 | 79.70 | 84.37 |
| 6 | QZ07E2-Hobi-like 11-109 Chimeric FC | 3.37 | 12.03 | 53.72 | 59.60 | 79.07 | 82.67 | 85.81 | 79.82 | 84.72 |
| 7 | QZ07E2-Hobi-like 11-109 Chimeric G25D FC | 2.44 | 0.71 | 54.87 | 62.76 | 84.63 | 85.81 | 89.38 | 87.01 | 87.02 |
| 8 | QZ07E2-WT 330 FC | 5.70 | 7.75 | 32.26 | 42.09 | 74.49 | 78.60 | 83.83 | 84.45 | 85.46 |
| 9 | Strict Control | 1.06 | 5.32 | 2.07 | 16.34 | 16.14 | 13.38 | 7.07 | 5.41 | 13.19 |

What we claim is:

1. A recombinant CSFV (classical swine fever virus) E2 protein in which a fragment comprising at least one of the amino acids defining the 6B8 epitope of the CSFV E2 protein is replaced by a corresponding fragment of the E2 protein from a pestivirus other than CSFV.

2. The recombinant CSFV E2 protein according to claim 1, wherein the replacement results in a mutated 6B8 epitope in the recombinant CSFV E2 protein to which the binding of the 6B8 monoclonal antibody or the antigen-binding fragment thereof is specifically inhibited.

3. The recombinant CSFV E2 protein according to claim 1 or 2, wherein the recombinant CSFV E2 protein is derived from a wildtype CSFV E2 protein from a strain selected from C-strain, QZ07, GD18 and GD191.

4. The recombinant CSFV E2 protein according to any one of claims 1-3, wherein the wildtype CSFV E2 protein comprises an amino acid sequence selected from SEQ ID NOs: 9-12.

5. The recombinant CSFV E2 protein according to any one of claims 1-4, wherein the 6B8 epitope of the CSFV E2 protein is specifically recognized and/or bound by the 6B8 monoclonal antibody which
(i) is produced by a hybridoma deposited at CCTCC under the accession number CCTCC C2018120, or
(ii) comprises a heavy chain variable region (VH) having an amino acid sequence as set forth in SEQ ID NO: 7 and a light chain variable region (VL) having an amino acid sequence as set forth in SEQ ID NO: 8, or
(iii) comprises the CDRs of the monoclonal antibody produced by a hybridoma deposited at CCTCC under the accession number CCTCC C2018120, or
(iv) comprises a VH CDR1 comprising the amino acid sequence set forth in SEQ ID NO:1, a VH CDR2 comprising the amino acid sequence set forth in SEQ ID NO:2, a VH CDR3 comprising the amino acid sequence set forth in SEQ ID NO:3, a VL CDR1 comprising the amino acid sequence set forth in SEQ ID NO:4, a VL CDR2 comprising the amino acid sequence set forth in SEQ ID NO:5, and a VL CDR3 comprising the amino acid sequence set forth in SEQ ID NO:6.

6. The recombinant CSFV E2 protein according to any one of claims 1-5, wherein the amino acids defining the 6B8 epitope of the CSFV E2 protein at least comprises the amino acid at position 14, position 22, position 24, positions 24/25, position 10, position 41 and/or position 64 of the CSFV E2 protein.

7. The recombinant CSFV E2 protein according to any one of claims 1-6, wherein the fragment comprising at least one of the amino acids defining the 6B8 epitope of the CSFV E2 protein is the B/C domain or a fragment thereof of the CSFV E2 protein.

8. The recombinant CSFV E2 protein according to any one of claims 1-7, wherein
i) the sequence of amino acid position 11 to amino acid position 38 of the CSFV E2 protein;
ii) the sequence of amino acid position 11 to amino acid position 39 of the CSFV E2 protein;
iii) the sequence of amino acid position 11 to amino acid position 56 of the CSFV E2 protein;
iv) the sequence of amino acid position 11 to amino acid position 80 of the CSFV E2 protein;
v) the sequence of amino acid position 11 to amino acid position 90 of the CSFV E2 protein;
vi) the sequence of amino acid position 11 to amino acid position 109 of the CSFV E2 protein; or
vii) the sequence of amino acid position 11 to amino acid position 110 of the CSFV E2 protein is replaced by a corresponding sequence of the E2 protein from a pestivirus other than CSFV.

9. The recombinant CSFV E2 protein according to any one of claims 1-8, wherein the pestivirus other than CSFV is selected from bovine viral diarrahea viruses comprising BVDV-2; border disease viruses comprising Switzerland, BDV-1, BDV-2, BDV-3, BDV-4, BDV-5, BDV-6, Chamois, Italy, Turkey, and Tunisian_sheep_virus (TSV); and atypical pestiviruses comprising Giraffe pestivirus, BVDV-3, Pronghorn antelope, Bungowannah virus and Norway rat pestivirus.

10. The recombinant CSFV E2 protein according to claim 9, wherein the pestivirus other than CSFV is selected from Giraffe pestivirus PG2 strain, BDV-2 Reindeer V60, Norway rat pestivirus isolate NrPV/NYC-D23, TSV9552, Bungowannah 6778, and BVDV3 Hobi-like Th/04 KhonKaen.

11. The recombinant CSFV E2 protein according to any one of claims 1-10, wherein the E2 protein from a pestivirus other than CSFV comprises the amino acid sequence selected from SEQ ID NOs: 21-27.

12. The recombinant CSFV E2 protein according to any one of claims 1-11, which comprises at least one amino acid residue at amino acid positions defining the 6B8 epitope of the CSFV E2 protein which specifically inhibit the binding of 6B8 monoclonal antibody to the CSFV E2 protein.

13. The recombinant CSFV E2 protein according to any one of claims 1-12,
i. wherein the recombinant CSFV E2 protein comprises the amino acid mutations at the amino acid positions as defined in tables 1 to 7,
ii. wherein the amino acid at position 24 of the recombinant CSFV E2 protein is R, D or I, with R being preferred,
iii. the amino acid at positions 24 and 25 of the recombinant CSFV E2 protein is R, D or I, with R being preferred, and D, K, L, N, R, T, V, E or P, with D, K, L, N, R, T and V being preferred, respectively,
iv. the amino acid at position 14 of the recombinant CSFV E2 protein is K, A, E, Q or R, with K being preferred;
v. the amino acid at position 22 of the recombinant CSFV E2 protein is A, R, Q, E, D, N, K, L, P, T, V or S, with A, Q, D, E, N and S being preferred,
vi. the amino acid at position 10 of the recombinant CSFV E2 protein is A or P,
vii. the amino acid at position 41 of the recombinant CSFV E2 protein is A, N or E, and/or
viii. the amino acid at position 64 of the recombinant CSFV E2 protein is A, S, E, D, G, H, T, L, P, K or W, with A, K, and W being preferred.

14. The recombinant CSFV E2 protein according to any one of claims 1-13, wherein the amino acid at position 24 of the recombinant CSFV E2 protein is R, D or I, with R being preferred, the amino acid at positions 24 and 25 of the recombinant CSFV E2 protein is R, D or I, with R being preferred, and D, K, L, N, R, T, V, E or P, with D, K, L, N, R, T and V being preferred, respectively, the amino acid at position 14 of the recombinant CSFV E2 protein is K, A, E, Q or R, with K being preferred, and the amino acid at position 22 of the recombinant CSFV E2 protein is A, R, Q, E, D, N, K, L, P, T, V or S, with A, Q, D, E, N and S being preferred.

15. The recombinant CSFV E2 protein according to claim 14, wherein the amino acid at position 24 of the recombinant CSFV E2 protein is R, the amino acid at positions 25 of the recombinant CSFV E2 protein is D, the amino acid at position 14 of the recombinant CSFV E2 protein is K, and the amino acid at position 22 of the recombinant CSFV E2 protein is A.

16. The recombinant CSFV E2 protein according to any one of claims 1 to 15, wherein the recombinant CSFV E2 protein comprises an amino acid sequence selected from the group consisting of SEQ ID NOs: 27-48 and 71-74.

17. The recombinant CSFV E2 protein according to any one of claims 1 to 16, wherein an Fc fragment, such as a swine Fc fragment is linked to the recombinant CSFV E2 protein; preferably the Fc fragment, such as a swine Fc fragment is linked to the C terminus of the E2 protein, preferably via a peptide linker.

18. The recombinant CSFV E2 protein according to claim 17, wherein the Fc fragment comprises an amino acid sequence of SEQ ID NO:20.

19. The recombinant CSFV E2 protein according to claim 17 or 18, wherein the peptide linker comprises an amino acid sequence selected from SEQ ID NOs: 16-19.

20. The recombinant CSFV E2 protein according to any one of claim 17-19, wherein the recombinant CSFV E2 protein comprises an amino acid sequence selected from the group consisting of SEQ ID NOs: 49-70 and 75-78; preferably selected from the group consisting of SEQ ID NOs: 53, 68 and 75; more preferably preferably selected from the group consisting of SEQ ID NOs: 53 and 75.

21. A recombinant CSFV E2 protein, wherein the recombinant CSFV E2 protein comprises an amino acid sequence of the E2 protein of CSFV field strain QZ07 with a fragment comprising at least one of the amino acids defining the 6B8 epitope being replaced by a corresponding fragment of the E2 protein of BVDV3 Hobi-like Th/04 KhonKaen strain.

22. The recombinant CSFV E2 protein according to claim 21,
wherein the sequence of amino acid position 11 to amino acid position 38 of the CSFV E2 protein is replaced by a corresponding sequence of the E2 protein from BVDV3 Hobi-like Th/04 KhonKaen strain, and the numbering of the amino acid position is determined based on the position numbers of the amino acid sequence of the E2 protein of CSFV field strain QZ07 shown by SEQ ID NO: 10; or
wherein the sequence of amino acid position 11 to amino acid position 39 of the CSFV E2 protein is replaced by a corresponding sequence of the E2 protein from BVDV3 Hobi-like Th/04 KhonKaen strain, and the numbering of the amino acid position is determined based on the position numbers of the amino acid sequence of the E2 protein of CSFV field strain QZ07 shown by SEQ ID NO: 10; or
wherein the sequence of amino acid position 11 to amino acid position 56 of the CSFV E2 protein is replaced by a corresponding sequence of the E2 protein from BVDV3 Hobi-like Th/04 KhonKaen strain, and the numbering of the amino acid position is determined based on the position numbers of the amino acid sequence of the E2 protein of CSFV field strain QZ07 shown by SEQ ID NO: 10; or
wherein the sequence of amino acid position 11 to amino acid position 80 of the CSFV E2 protein is replaced by a corresponding sequence of the E2 protein from BVDV3 Hobi-like Th/04 KhonKaen strain, and the numbering of the amino acid position is determined based on the position numbers of the amino acid sequence of the E2 protein of CSFV field strain QZ07 shown by SEQ ID NO: 10; or
wherein the sequence of amino acid position 11 to amino acid position 90 of the CSFV E2 protein is replaced by a corresponding sequence of the E2 protein from BVDV3 Hobi-like Th/04 KhonKaen strain, the numbering of the amino acid position is determined based on the position numbers of the amino acid sequence of the E2 protein of CSFV field strain QZ07 shown by SEQ ID NO: 10; or
wherein the sequence of amino acid position 11 to amino acid position 109 of the CSFV E2 protein is replaced by a corresponding sequence of the E2 protein from BVDV3 Hobi-like Th/04 KhonKaen strain, and the numbering of the amino acid position is determined based on the position numbers of the amino acid sequence of the E2 protein of CSFV field strain QZ07 shown by SEQ ID NO: 10.

23. The recombinant CSFV E2 protein according to claim 22, wherein compared to the corresponding fragment of the E2 protein of BVDV3 Hobi-like Th/04 KhonKaen strain, the recombinant CSFV E2 protein further comprises at least one amino acid mutation at positions 10, 14, 22, 24, 25, 24/25, 41 and/or 64; preferably, the recombinant CSFV E2 protein further comprises the amino acid mutations at the amino acid positions as defined in tables 1 to 7; preferably wherein the recombinant CSFV E2 protein further comprises one single amino acid mutation at position 14, 24, 25 or 64 of the recombinant CSFV E2 protein.

24. The recombinant CSFV E2 protein according to claim 23, wherein
the amino acid at position 14 of the recombinant CSFV E2 protein is K, A, E, Q or R, with K being preferred;
the amino acid at position 64 of the recombinant CSFV E2 protein is A, S, E, D, G, H, T, L, P, K or W, with K being preferred;
the amino acid at position 24 of the recombinant CSFV E2 protein is R, D or I, with R being preferred;
the amino acid at position 25 of the recombinant CSFV E2 protein is D, K, L, N, R, T, V, E or P, with D being preferred;
the amino acid at position 25 of the recombinant CSFV E2 protein is D, K, L, N, R, T, V, E or P, with N being preferred; or
the amino acid at position 25 of the recombinant CSFV E2 protein is D, K, L, N, R, T, V, E or P, with R being preferred.

25. The recombinant CSFV E2 protein according to any one of claims 21-24, wherein an Fc fragment, such as a swine Fc fragment, is linked to the recombinant CSFV E2 protein; preferably the Fc fragment, such as a swine Fc fragment, is linked to the C terminus of the E2 protein, preferably via a peptide linker.

26. The recombinant CSFV E2 protein according to claim 25, wherein the Fc fragment comprises an amino acid sequence of SEQ ID NO: 20; and/or the peptide linker comprises an amino acid sequence selected from SEQ ID NOs: 16-19.

27. The recombinant CSFV E2 protein according to claim 26, wherein the recombinant CSFV E2 protein comprises or consists of an amino acid sequence selected from the group consisting of SEQ ID NOs: 96-101; preferably the recombinant CSFV E2 protein comprises or consists of an amino acid sequence shown by SEQ ID NO: 96.

28. A recombinant nucleic acid coding for the recombinant CSFV E2 protein according to any one of claims 1 to 27.

29. A vector comprising the nucleic acid of claim 28.

30. A host cell comprising the nucleic acid of claim 28 or the vector of claim 29, preferably, the host cell is a mammalian cell, such as a CHO cell.

31. A method for producing the recombinant CSFV E2 protein according to any one of claims 1-27, comprising
(i) culturing the host cell of claim 30 under conditions suitable for the expression of the CSFV E2 protein, and
(ii) isolating and optionally purifying the CSFV E2 protein.

32. A recombinant CSFV (classical swine fever virus) comprising the recombinant CSFV E2 protein of any one of claims 1-27.

33. The recombinant CSFV of claim 32, wherein the recombinant CSFV is attenuated.

34. An immunogenic composition comprising the recombinant CSFV E2 protein according to any one of claims 1 to 27, the recombinant nucleic acid according to claim 28, the vector according to claim 29, and/or the recombinant CSFV of claim 32 or 33.

35. The immunogenic composition according to claim 34, wherein said immunogenic composition is a vaccine, such as a marker vaccine or a DIVA (differentiation between infected and vaccinated animals) vaccine.

36. An immunogenic composition according to claim 34 or 35 for use in a method of preventing and/or treating diseases associated with CSFV in an animal, the method comprising the step of administering the immunogenic composition according to claim 34 or 35 to an animal in need thereof.

37. A method of preventing and/or treating diseases associated with CSFV in an animal, the method comprising the step of administering the immunogenic composition according to claim 34 or 35 to an animal in need thereof.

38. A method of differentiating animals infected with CSFV from animals vaccinated with the immunogenic composition of any one of claim 34 or 35, comprising
a) obtaining a sample, and
b) testing said sample in an immuno test.

39. The method according to claim 38, wherein the immuno test comprises testing whether an antibody specifically recognizing the 6B8 epitope of the CSFV E2 protein or an antigen-binding fragment thereof can bind to the CSFV E2 protein in the sample.

40. The method according to claim 38 or 39, wherein the immuno test comprises testing whether an antibody specifically recognizing a 6B8 epitope of the CSFV E2 protein is present in the sample, and/or testing whether an antibody specifically recognizing a mutated 6B8 epitope of the recombinant CSFV E2 protein is present in the sample.

41. The method according to any one of claims 38-40, wherein the immuno test is an EIA (enzyme immunoassay) or ELISA (enzyme linked immunosorbent assay), preferably a double competitive ELISA.

42. The method according to any one of claims 39-41, wherein the antibody specifically recognizing the 6B8 epitope
(i) is produced by a hybridoma deposited at CCTCC under the accession number CCTCC C2018120, or
(ii) comprises a heavy chain variable region (VH) having an amino acid sequence as set forth in SEQ ID NO: 7 and a light chain variable region (VL) having an amino acid sequence as set forth in SEQ ID NO: 8, or
(iii) comprises the CDRs of the monoclonal antibody produced by a hybridoma deposited at CCTCC under the accession number CCTCC C2018120, or
(iv) comprises a VH CDR1 comprising the amino acid sequence set forth in SEQ ID NO:1, a VH CDR2 comprising the amino acid sequence set forth in SEQ ID NO:2, a VH CDR3 comprising the amino acid sequence set forth in SEQ ID NO:3, a VL CDR1 comprising the amino acid sequence set forth in SEQ ID NO:4, a VL CDR2 comprising the amino acid sequence set forth in SEQ ID NO:5, and a VL CDR3 comprising the amino acid sequence set forth in SEQ ID NO:6.

43. A kit for differentiating animals infected with CSFV from animals vaccinated with the immunogenic composition of any one of claim 34 or 35, which comprises an antibody specifically recognizing the 6B8 epitope of the CSFV E2 protein or an antigen-binding fragment thereof.

44. A method for producing the recombinant CSFV E2 protein according to any one of claims 1 to 27, in CHO cells, said method comprises
a) adapting CHO cells to high-density suspension culture in a medium;
b) transfecting the adapted CHO cells from step a) with a mammalian expression vector comprising the nucleic acid molecule encoding the recombinant CSFV E2 protein according to any one of claims 1 to 27,
c) culturing the transfected CHO cells from step b) under conditions suitable for the expression of the recombinant CSFV E2 protein;
d) harvesting and optionally purifying the recombinant CSFV E2 protein.

45. A method for producing the recombinant CSFV E2 protein according to any one of claims 1 to 27, in CHO cells, said method comprises
a) growing CHO cells to high-density suspension culture in a medium;
b) transfecting the CHO cells from step a) with a mammalian expression vector comprising the nucleic acid molecule encoding the recombinant CSFV E2 protein according to any one of claims 1 to 27,
c) culturing the transfected CHO cells from step b) under conditions suitable for the expression of the recombinant CSFV E2 protein; and
d) harvesting and optionally purifying the recombinant CSFV E2 protein.

46. The method of claim 44 or 45, wherein in step c), the transfected CHO cells are cultured at about 32-37° C., preferably about 32° C.

47. The method of any one of claims 44-46, wherein in step c), the transfected CHO cells are cultured with a humidified atmosphere of about 5-8% CO₂.

48. The method of any one of claims 44-47, wherein the recombinant CSFV E2 protein is harvested about 2-14 days post transfection, for example, about 4-12 days post transfection, or about 8-10 days post transfection.
